SEQUENCE LISTING (6 sequences)

SEQ ID NO: 1 - protein, 812 aa, Homo sapiens
MGRKLDPTKEKRGPGRKARKQKGAETELVRFLPAVSDENSKRLSSRARKRAAKRRLGSVEAPKTNKSPEAKPLPGKLPKGISAGAVQTAGKKGPQSLFNAPRGKKRPAPGSDEEEEEEDSEEDGMVNHGDLWGSEDDADTVDDYGADSNSEDEEEGEALLPIERAARKQKAREAAAGIQWSEEETEDEEEEKEVTPESGPPKVEEADGGLQINVDEEPFVLPPAGEMEQDAQAPDLQRVHKRIQDIVGILRDFGAQREEGRSRSEYLNRLKKDLAIYYSYGDFLLGKLMDLFPLSELVEFLEANEVPRPVTLRTNTLKTRRRDLAQALINRGVNLDPLGKWSKTGLVVYDSSVPIGATPEYLAGHYMLQGASSMLPVMALAPQEHERILDMCCAPGGKTSYMAQLMKNTGVILANDANAERLKSVVGNLHRLGVTNTIISHYDGRQFPKVVGGFDRVLLDAPCSGTGVISKDPAVKTNKDEKDILRCAHLQKELLLSAIDSVNATSKTGGYLVYCTCSITVEENEWVVDYALKKRNVRLVPTGLDFGQEGFTRFRERRFHPSLRSTRRFYPHTHNMDGFFIAKFKKFSNSIPQSQTGNSETATPTNVDLPQVIPKSENSSQPAKKAKGAAKTKQQLQKQQHPKKASFQKLNGISKGADSELSTVPSVTKTQASSSFQDSSQPAGKAEGIREPKVTGKLKQRSPKLQSSKKVAFLRQNAPPKGTDTQTPAVLSPSKTQATLKPKDHHQPLGRAKGVEKQQLPEQPFEKAAFQKQNDTPKGPQPPTVSPIRSSRPPPAKRKKSQSRGNSQLLLS

SEQ ID NO: 2 - protein, 767 aa, Homo sapiens
MGRRSRGRRLQQQQRPEDAEDGAEGGGKRGEAGWEGGYPEIVKENKLFEHYYQELKIVPEGEWGQFMDALREPLPATLRITGYKSHAKEILHCLKNKYFKELEDLEVDGQKVEVPQPLSWYPEELAWHTNLSRKILRKSPHLEKFHQFLVSETESGNISRQEAVSMIPPLLLNVRPHHKILDMCAAPGSKTTQLIEMLHADMNVPFPEGFVIANDVDNKRCYLLVHQAKRLSSPCIMVVNHDASSIPRLQIDVDGRKEILFYDRILCDVPCSGDGTMRKNIDVWKKWTTLNSLQLHGLQLRIATRGAEQLAEGGRMVYSTCSLNPIEDEAVIASLLEKSEGALELADVSNELPGLKWMPGITQWKVMTKDGQWFTDWDAVPHSRHTQIRPTMFPPKDPEKLQAMHLERCLRILPHHQNTGGFFVAVLVKKSSMPWNKRQPKLQGKSAETRESTQLSPADLTEGKPTDPSKLESPSFTGTGDTEIAHATEDLENNGSKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKILLTQENPFFRKLSSETYSQAKDLAKGSIVLKYEPDSANPDALQCPIVLCGWRGKASIRTFVPKNERLHYLRMMGLEVLGEKKKEGVILTNESAASTGQPDNDVTEGQRAGEPNSPDAEEANSPDVTAGCDPAGVHPPR

SEQ ID NO: 3 - DNA, 42 bp, synthetic construct
  source 1..42: mol_type = other DNA
cctatctttactcgcgtccaattagaattctacgaattcatc

SEQ ID NO: 4 - DNA, 42 bp, synthetic construct
  source 1..42: mol_type = other DNA
aatcttaagatgcttaagtagtagttcattccaatcgactct

SEQ ID NO: 5 - DNA, 21 bp, synthetic construct
  source 1..21: mol_type = other DNA
ttagaattctacgaattcatc

SEQ ID NO: 6 - DNA, 42 bp, synthetic construct
  source 1..42: mol_type = other DNA
tctcagctaaccttacttgatgatgaattcgtagaattctaa

(19) United States
(12) Patent Application Publication
(10) Pub. No.: US 2026/0306459 A1
(43) Pub. Date: Oct. 8, 2026

(54) INHIBITORS OF NSUN1- AND NSUN2-MEDIATED ACTIVE STRUCTURES

(71) Applicant: THE UNIVERSITY OF CHICAGO, Chicago, IL (US)

(72) Inventors: Jason X. CHENG, Chicago, IL (US); Rick L. STEVENS, Chicago, IL (US)

(21) Appl. No.: 19/478,964

(22) PCT Filed: May 3, 2024
(86) PCT No.: PCT/US2024/027873

§ 371 Date: Oct. 27, 2025

Related U.S. Application Data
(60) Provisional application No. 63/499,811, filed on May 3, 2023.

(51) Int. Cl. A61K 31/4196; A61K 31/4184; A61K 31/4453; A61P 35/00

(52) U.S. Cl. CPC A61K 31/4196; A61K 31/4184; A61K 31/4453; A61P 35/00

(57) ABSTRACT

Provided herein are methods and compositions for the inhibition of NSUN1 and/or NSUN2 interactions with certain binding partners, such as bromodomain-containing protein 4 (BRD4) and elongating RNA polymerase II (eRNAPII). Also provided herein are methods and compositions for treatment of drug resistant cancer, particularly venetoclax and/or 5-AZA resistant cancer.

### RELATED APPLICATIONS

This application claims the benefit of priority of U.S. Provisional Patent Application Ser. No. 63/499,811 filed May 3, 2023, the contents of which are hereby incorporated by reference in their entirety.

### SEQUENCE LISTING

The instant application contains a Sequence Listing which has been submitted in ST26 format and is hereby incorporated by reference in its entirety. Said ST26 copy, created on May 3, 2024, is named ARCD_0797_Sequence_Listing.xml and is 7,738 bytes in size.

### FIELD

The disclosure relates at least to the fields of medicine, biomedical research, epigenetic regulation, and cancer therapy.

### INTRODUCTION AND BACKGROUND

Development of drug resistance is a significant problem in cancer therapy, particularly leukemias. Greater than 90% of cancer mortality cases are contributed to by drug resistance (see e.g., Bukowski K, et al. Int J Mol Sci. 2020; Saliba A N, et al. Cancer Drug Resist. 2021; and/or Ong F, et al. Cancer Drug Resist. 2022). Almost 100% of Myelodysplastic Syndrome (MDS) and Acute Myeloid Leukemia (AML) patients treated with DNA hypomethylating agents (HMAs) eventually develop drug resistance (see e.g., Stomper J., et al., Leukemia. 2021). Venetoclax is a common first line treatment option for MDS/AML cases, however, ~30-40% of MDS/AML have primary venetoclax resistance, and >60% of AML patients develop venetoclax acquired resistance when treated with venetoclax-based therapy (see e.g., Dhakal P., et al., Blood Rev. 2023). It is difficult to predict or overcome resistance to venetoclax-based therapies and/or combination therapies, such as venetoclax coupled with 5-Azacytidine (5-AZA). In many cases, the cancers develop multifaced drug resistance mechanisms.

There remains a pressing need for development of additional therapies to overcome tumor primary and acquired drug resistance to treatment options such as venetoclax and/or 5-AZA.

### SUMMARY

Provided herein are at least compositions and methods for treating cancer, particularly those that have or are likely to develop drug resistance. In particular, in certain aspects, provided herein are compositions and methods for targeting epigenetic regulators that can contribute to drug resistance, particularly venetoclax and/or 5-AZA resistant tumors. Provided herein are new small-molecule compounds and new methods of use of small molecule compounds for inhibition of NSUN1 and/or NSUN2 mediated structures, such as but not limited to, active chromatin structures (ACS). In certain aspects, provided herein are compositions and methods for inhibition of NSUN1/2 mediated mitochondrial transcriptional complexes, nuclear transcriptional complexes, and/or cytoplasmic translational complexes.

As described herein, the inventors leveraged the Argonne AI supercomputing drug design to design novel functional drug screening to identify small-molecule inhibitors that target NSUN1- and/or NSUN2-ACS (NSUN1/2-ACS inhibitors) to overcome cancer drug resistance. Initial functional screening identified numerous compounds that could successfully target NSUN1 and/or NSUN2 to inhibit the growth of drug-resistant acute monocytic leukemia SC cells. Drug resistance is a major obstacle for the successful treatment of cancer, and the identification of new treatment options for overcoming drug-resistance are sorely needed.

It is specifically contemplated that any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Any aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects set forth in examples and figures described herein are also aspect that may be implemented in the context of aspect discussed elsewhere in a different example or elsewhere in the application, such as in the Summary, Detailed Description, Claims, and Brief Description of the Drawings.

Other objects, features and advantages of the present inventions will become apparent from the following detailed description. It should be understood, however, that the detailed description and the specific examples, while indicating specific embodiments of the disclosure, are given by way of illustration only, since various changes and modifications within the spirit and scope of the inventions described herein will become apparent to those skilled in the art from this detailed description.

Screening for additional inhibitory molecules & identification of additional suitable modifications to the inhibitory molecules identified are ongoing.

In some aspects, functional drug screening of other cancers with different cell lineages may be appropriate as cells may exhibit cell lineage-associated NSUN1/2-ACS mediated drug resistance, (e.g., see Tables 2, and 4-6—Exemplary identified small-molecule inhibitors that target NSUN1 and/or NSUN2)

The inventors first discovered NSUN1/2-ACS as potentially controlling and/or mediating cell lineage-associated azacitidine resistance in leukemia in Cheng, J. X. et al., Nature Communications 2018, which is incorporated herein by reference in its entirety for the purposes described herein.

While not being limited by theory, the data presented herein demonstrated that NSUN1/2-ACS also regulated cell lineage-associated venetoclax resistance in leukemia cells. Mechanistically, specific RNA cytosine methyltransferases (RCMTs), namely NSUN1 and NSUN2, directly interact with the bromodomain-containing protein 4 (BRD4) and elongating RNA polymerase II (eRNAPII) to form distinct ACS at nascent RNAs, and such NSUN1- and NSUN2-mediated ACS (NSUN1/2-ACS) control drug (e.g., venetoclax and/or azacitidine) resistance in myelodysplastic syndrome (MDS) and acute myeloid leukemia (AML).

The studies descried herein demonstrated the usefulness of NSUN1/2-ACS as novel biomarkers for predicting drug resistance in MDS/AML, and as novel targets for overcoming drug resistance in cancers (e.g., MDS/AML, etc.). (e.g., see FIG. 2—A schematic illustration of drug-resistant NSUN1/2-ACS.)

The inventors have also developed NSUN1/2-ACS-targeting technologies for rapid prediction and in-depth analysis of cancer drug resistance as well as high-throughput screening of drug/compound libraries to identify inhibitors that target NSUN1/2 and their associated ACS (e.g., see FIGS. 4-9, The data demonstrated NSUN1/2-ACS-targeting proximity ligation rolling cycle amplification-coupled flow cytometry (NSUN1/2-ACS/PL-RCA-FCM), and Fluorescence quenching release assay (NSUN1/2-ACS/FQRA).

The inventors have discovered that inhibition of nascent RNA synthesis can act as a superfast biomarker for predicting drug response and resistance in leukemia cells. Furthermore, that cells exhibited unique patterns of RNA synthesis in response to specific drugs, and showed drug-specific signatures of RNA synthesis inhibition, (e.g., see FIG. 8, the data demonstrated drug-specific responsive and resistant signatures of nascent RNA synthesis inhibition by 5-ethynyl uridine click chemistry (EC)-coupled flow cytometry (EC-FCM) in leukemia cells.

The NSUN1/2-ACS targeting biomarkers, technologies and small-molecule inhibitors described herein can greatly improve diagnosis and/or treatment of cancers, such as but not limited to hematological cancers, e.g., leukemia, and other human diseases associated with elevated expression levels of NSUN1 and/or NSUN2. In general, elevated expression levels of NSUN1 and/or NSUN2 are correlated with significantly shorter survival in almost all cancer subtypes (see e.g., Uhlen, M., et al., Science, 2017, which is incorporated herein by reference in its entirety for the purposes described herein).

To date, the inventors are aware of no NSUN1/2-targeting diagnostics and/or therapeutics that have been developed. By targeting NSUN1/2-ACS, the biomarkers, technologies, and identified small molecule compounds described herein can be utilized to not only quickly predict drug resistance, but also identify therapeutic alternatives and methods of treatment for overcoming drug resistant cancers.

The immediate disclosure provides compositions and methods for NSUN1/2-targeting diagnostics and/or therapeutics, which have not previously been developed, e.g., the inventors have identified small-molecule NSUN1/2-ASC inhibitors that have novel biological anticancer activities acting through newly described action mechanisms, identified through a novel AI/supercomputing and RNA epigenetic functional approach. In addition, the inventors have developed assays such as fluorescence quenching complex and NSUN1/2-ACS-targeting NSUN1/2-ACS/FQRA plate assay for high-throughput drug testing and screening. The inventors have also modified previous PL-RCA-FCM assays for efficiently measuring NSUN1/2-ACS, and developed the concept of drug-specific signatures/patterns of nascent RNA synthesis inhibition and the EC-FCM and EC-RNA-sequencing for capturing such signatures.

Certain aspects of the present disclosure are characterized through the following enumerated aspects.

Aspect 1 a method of treating a disease in an individual, the method comprising the step of administering to the individual a therapeutically effective amount of any one or more of an inhibitor of NSUN1 and/or NSUN2 interactions with Active Chromatin Site (ACS) associated proteins and/or complexes (NSUN1/2-ACS inhibitor).

Aspect 2 is the method of aspect 1, wherein the NSUN1/2-ACS inhibitor comprises NSUN2-11, NSUN2-37, NSUN2-35, NSUN1-17.16, NSUN1-11, NSUN1-67, NSUN2-34, NSUN2-98, NSUN2-38, NSUN2-39, NSUN1-19, NSUN1-9, NSUN1-10, NSUN1-17, NSUN1-17.13, NSUN1-24, NSUN1-25, NSUN1-27, NSUN1-30, NSUN1-31, NSUN1-54, NSUN1-64, NSUN1-73, NSUN1-74, NSUN2-3, NSUN2-10, NSUN2-17, NSUN2-18, NSUN2-19, NSUN2-20, NSUN2-21, NSUN2-64, and/or a derivative thereof.

Aspect 3 is the method of aspect 1 or 2, wherein the NSUN1/2-ACS inhibitor comprises NSUN2-11, NSUN2-37, NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-35, and/or a derivative thereof.

Aspect 4 is the method of aspect 3, wherein the NSUN1/2-ACS inhibitor comprises a water soluble derivative of compounds NSUN2-11, NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-37, and/or NSUN2-35.

Aspect 5 is the method of aspect 1 or 2, wherein the NSUN1/2-ACS inhibitor comprises NSUN1-24, NSUN1-14, NSUN1-16, NSUN1-17, NSUN1-17.3, NSUN1-19, NSUN1-73, and/or a derivative thereof.

Aspect 6 is the method of aspect 5, wherein the NSUN1/2-ACS inhibitor comprises a water soluble derivative of compounds NSUN1-24, NSUN1-14, NSUN1-16, NSUN1-17, NSUN1-17.3, NSUN1-19, and/or NSUN1-73.

Aspect 7 is the method of aspect 1 or 2, wherein the NSUN1/2-ACS inhibitor comprises compounds NSUN1-17.16, NSUN1-11, NSUN1-67, NSUN2-11, NSUN2-37, NSUN2-35, and/or a derivative thereof.

Aspect 8 is the method of aspect 7, wherein the NSUN1/2-ACS inhibitor comprises a water soluble derivative of compounds NSUN1-17.16, NSUN1-11, NSUN1-67, NSUN2-11, NSUN2-37, and/or NSUN2-35.

Aspect 9 is the method of any one of aspects 1-8, wherein the disease is cancer.

Aspect 10 is the method of aspect 9, wherein the cancer is a blood cancer or a solid cancer.

Aspect 11 is the method of aspect 9 or 10, wherein the cancer is a leukemia and/or a myelodysplastic syndrome (MDS).

Aspect 12 is the method of any one of aspects 9-11, wherein the cancer is a drug-resistant cancer.

Aspect 13 is the method of any one of aspects 9-12, wherein the cancer is resistant to venetoclax-based and/or 5-azacytidine-based therapies.

Aspect 14 is the method of any one of aspects 1-13, wherein the NSUN1/2-ACS inhibitor binds to at least the A-460 binding module of NSUN1.

Aspect 15 is the method of any one of aspects 1-14, wherein the NSUN1/2-ACS inhibitor binds to at least the A-316 binding module of NSUN1.

Aspect 16 is the method of any one of aspects 1-15, wherein the NSUN1/2-ACS inhibitor binds to at least the A-691 binding module of NSUN2.

Aspect 17 is the method of any one of aspects 1-16, wherein the NSUN1/2-ACS inhibitor binds to at least the A-580 binding module of NSUN2.

Aspect 18 is the method of any one of aspects 1-17, wherein the NSUN1/2-ACS inhibitor binds to at least the A-271 binding module of NSUN2.

Aspect 19 is the method of any one of aspects 1-18, wherein the NSUN1/2-ACS inhibitor binds to at least the A-279 binding module of NSUN2.

Aspect 20 is the method of any one of aspects 1-19, wherein the NSUN1/2-ACS inhibitor binds to at least two binding modules of NSUN1 and/or NSUN2 selected from the group consisting of NSUN1 A-460, NSUN1 A-316, NSUN1 A-371, NSUN1 A-219, NSUN2 A-700, NSUN2 A-691, NSUN2 A-580, NSUN2 A-301, NSUN2-A279, NSUN2 A-271, NSUN2 A-202, NSUN2 A-162, and/or NSUN2 A-89.

Aspect 21 is the method of any one of aspects 1-20, wherein the NSUN1/2-ACS inhibitor binds to at least two binding modules of NSUN1 and/or NSUN2 selected from the group consisting of NSUN1 A-460, NSUN1 A-316, NSUN2 A-691, NSUN2 A-580, NSUN2 A-271, and NSUN2 A-279.

Aspect 22 is the method of any one of aspects 1-21, wherein the NSUN1/2-ACS inhibitor binds to more than two binding modules of NSUN1 and/or NSUN2.

Aspect 23 is the method of any one of aspects 14-22, wherein the NSUN1/2-ACS inhibitor binds to at least one binding module of NSUN1 and at least one binding module of NSUN2.

Aspect 24 is the method of any one of aspects 1-23, wherein the NSUN1/2-ACS inhibitor comprises, consists essentially of, or consists of NSUN1-11 and/or a derivative thereof.

Aspect 25 is the method of any one of aspects 1-23, wherein the NSUN1/2-ACS inhibitor comprises, consists essentially of, or consists of NSUN1-67 and/or a derivative thereof.

Aspect 26 is the method of any one of aspects 1-23, wherein the NSUN1/2-ACS inhibitor comprises, consists essentially of, or consists of NSUN1-17.16 and/or a derivative thereof.

Aspect 27 is the method of any one of aspects 1-23, wherein the NSUN1/2-ACS inhibitor comprises, consists essentially of, or consists of NSUN2-11 and/or a derivative thereof.

Aspect 28 is the method of any one of aspects 1-23, wherein the NSUN1/2-ACS inhibitor comprises, consists essentially of, or consists of NSUN2-37 and/or a derivative thereof.

Aspect 29 is the method of any one of aspects 1-23, wherein the NSUN1/2-ACS inhibitor comprises, consists essentially of, or consists of NSUN2-35 and/or a derivative thereof.

Aspect 30 is the method of any one of aspects 9-29, wherein the NSUN1/2-ACS inhibitor reduces cancer cell proliferation.

Aspect 31 is the method of any one of aspects 1-30, wherein the NSUN1/2-ACS inhibitor has substantively no effect on NSUN1/2-BRD4 interactions.

Aspect 32 is the method of any one of aspects 1-31, wherein the NSUN1/2-ACS inhibitor has substantively no effective on NSUN1/2-m5C interactions.

Aspect 33 is the method of any one of aspects 1-32, wherein the NSUN1/2-ACS inhibitor substantively inhibits NSUN1/2-RNAPII interactions.

Aspect 34 is the method of any one of aspects 1-33, wherein the NSUN1/2-ACS inhibitor substantively inhibits NSUN1/2-c-MYC interactions.

Aspect 34.1 is the method of any one of aspects 1-34, wherein the NSUN1/2-ACS inhibitor substantively inhibits NSUN1/2-POLRMT (mitochondrial RNA polymerase) interactions.

Aspect 34.2 is the method of any one of aspects 1-34.1, wherein the NSUN1/2-ACS inhibitor substantively inhibits NSUN1/2-MTIF3 (mitochondrial translational initiation factor 3) interactions.

Aspect 35 is the method of any one of aspects 1-34.2, wherein the NSUN1/2-ACS inhibitor is part of a combination therapy.

Aspect 36 is the method of aspect 35, wherein the combination therapy comprises venetoclax-based and/or 5-azacytidine-based therapies.

Aspect 37 is the method of any one of aspects 1-36, wherein the ACS associated complex comprises an elongating RNA-Polymerase-II (eRNAPII) complex.

Aspect 38 is the method of any one of aspects 1-37, wherein the NSUN1/2-ACS inhibitor functions in a dose-dependent manner.

Aspect 39 is the method of any one of aspects 1-37, comprising administering the NSUN1/2-ACS inhibitor at a concentration of 0.001 to 1,000 M/kg weight of the individual.

Aspect 40 is the method of any one of aspects 1-39, comprising administering the NSUN1/2-ACS inhibitor at a concentration of 1 to 100 μM/kg weight of the individual.

Aspect 41 is the method of any one of aspects 1-40, comprising administering the NSUN1/2-ACS inhibitor at a concentration of 1 to 10 μM/kg weight of the individual.

Aspect 42 is the method of any one of aspects 1-41, wherein the NSUN1/2-ACS inhibitor comprises a compound of formula I

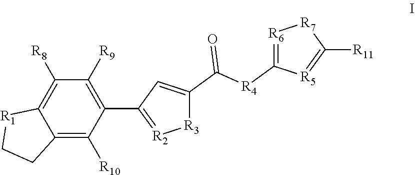

wherein:
   R₁ is CH₂, NH, O, or S;
   R₂ is CH or N;
   R₃ is CH₂, NH, O, or S;
   R₄ is NH or CH₂;
   R₅ is N or CH;
   R₆ is N or CH;
   R₇ is CH₂, NH, O, or S;
   R₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 43 is the method of aspect 42, wherein R1 is O.

Aspect 44 is the method of aspect 42, wherein R4 is NH.

Aspect 45 is the method of aspect 42, wherein R11 is alkyl.

Aspect 46 is the method of aspect 45, wherein R11 is isopropyl.

Aspect 47 is the method of aspect 42, wherein R3 and R7 are NH.

Aspect 48 is the method of aspect 42, wherein R2, R5, and R6 are N.

Aspect 49 is the method of aspect 42, wherein the compound of formula I is further defined as

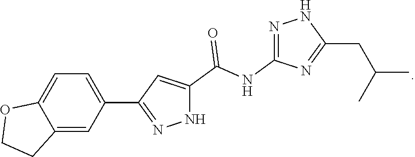 [SMILES: CC(C)Cc1nc(NC(=O)c2cc(-c3ccc4c(c3)CCO4)n[nH]2)n[nH]1]

Aspect 50 is the method of any one of aspects 1-41, wherein the NSUN1/2-ACS inhibitor comprises a compound of formula II:

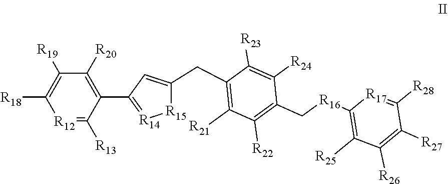

wherein:
   R₁₂ is CH or N;
   R₁₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄ is CH₂, NH, O, or S;
   R₁₅ is CH or N;
   R₁₆ is CH₂, NH, O, or S;
   R₁₇ is CH or N;
   R₁₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; or an enantiomer, diastereomer, racemic mixture, or salt thereof.

Aspect 51 is the method of aspect 50, wherein R14 and R16 are O.

Aspect 52 is the method of aspect 50, wherein R12, R15, and R17 are N.

Aspect 53 is the method of aspect 50, wherein R12 is NH2.

Aspect 54 is the method of aspect 50, wherein the compound of formula II is further defined as

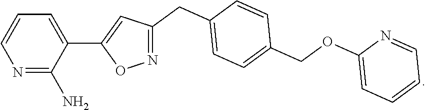 [SMILES: Nc1ncccc1-c1cc(Cc2ccc(COc3ccccn3)cc2)no1]

Aspect 55 is the method of any one of aspects 1-41, wherein the NSUN1/2-ACS inhibitor comprises a compound of formula III:

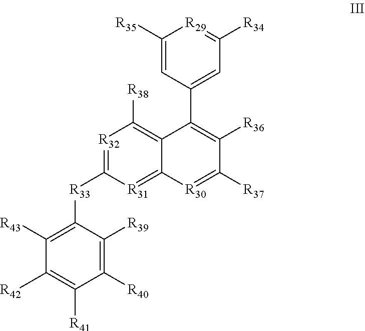

wherein:
   R₂₉ is N or CH;
   R₃₀ is N or CH;
   R₃₁ is N or CH;
   R₃₂ is N or CH;
   R₃₃ is CH₂, NH, O, or S;
   R₃₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₄₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₄₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₄₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₄₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; or an enantiomer, diastereomer, racemic mixture, or salt thereof.

Aspect 56 is the method of aspect 55, wherein R30, R31, R32, and R33 are N.

Aspect 57 is the method of aspect 55, wherein R38 is OH.

Aspect 58 is the method of aspect 55, wherein R41 is a halide.

Aspect 59 is the method of aspect 58, wherein R41 is —Cl.

Aspect 60 is the method of aspect 55, wherein R33 is NH.

Aspect 61 is the method of aspect 55, wherein the compound of formula III is further defined as

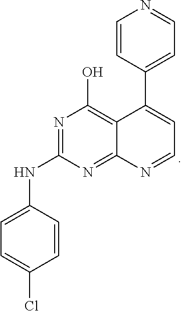 [SMILES: Oc1nc(Nc2ccc(Cl)cc2)nc2nccc(-c3ccncc3)c12]

Aspect 62 is the method of any one of aspects 1-41, wherein the NSUN1/2-ACS inhibitor comprises a compound of formula IV:

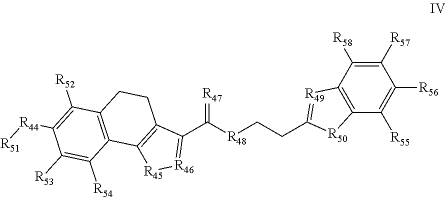

wherein:
   R₄₄ is O, S, NH, or CH₂
   R₄₅ is CH₂, NH, O, or S
   R₄₆ is CH or N;
   R₄₇ is NH, N-alkyl, O, or S;
   R₄₈ is CH₂ or NH;
   R₄₉ is CH or N;
   R₅₀ is CH₂, NH, O, or S;
   R₅₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and R₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 63 is the method of aspect 62, wherein R45, R48, and R50 are NH.

Aspect 64 is the method of aspect 62, wherein R44 and R47 are O.

Aspect 65 is the method of aspect 62, wherein R51 and R57 are alkyl.

Aspect 66 is the method of aspect 62, wherein R51 is methyl.

Aspect 67 is the method of aspect 62, wherein R57 is methyl.

Aspect 68 is the method of aspect 62, wherein the compound of formula IV is further defined as

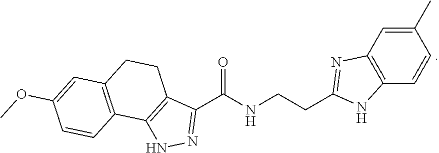

Aspect 69 is the method of any one of aspects 1-41, wherein the NSUN1/2-ACS inhibitor comprises a compound of formula V:

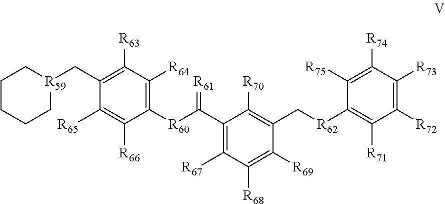

wherein:
R₅₉ is CH or N;
R₆₀ is CN₂, NH, O, or S;
R₆₁ is O or S;
R₆₂ is CH₂, NH, O, or S;
R₆₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 70 is the method of aspect 69, wherein R59 is N.

Aspect 71 is the method of aspect 69, wherein R60 is NH.

Aspect 72 is the method of aspect 69, wherein R51 is a halogen.

Aspect 73 is the method of aspect 72, wherein R51 is —Br.

Aspect 74 is the method of aspect 69, wherein R62 is O.

Aspect 75 is the method of aspect 69, wherein the compound of formula V is further defined as

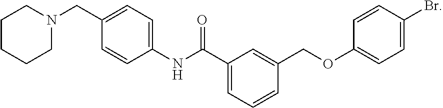 [SMILES: O=C(Nc1ccc(CN2CCCCC2)cc1)c1cccc(COc2ccc(Br)cc2)c1]

Aspect 76 is the method of any one of aspects 1-41, wherein the NSUN1/2-ACS inhibitor comprises a compound of formula VI:

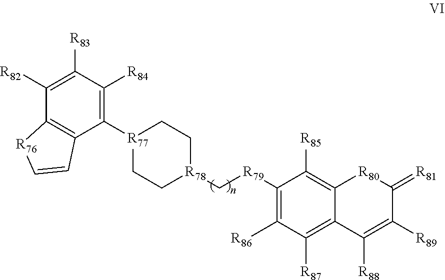

wherein:
   R₇₆ is CH₂, NH, O, or S;
   R₇₇ is CH or N;
   R₇₈ is CH or N;
   R₇₉ is CH₂, NH, S, or O;
   R₈₀ is CH₂, NH, O, or S;
   R₈₁ is CH₂, NH, or O;
   R₈₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₈₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 77 is the method of aspect 76, wherein R76 is S.

Aspect 78 is the method of aspect 76, wherein R77 and R78 are N.

Aspect 79 is the method of aspect 76, wherein R80 is NH.

Aspect 80 is the method of aspect 76, wherein R79 is O.

Aspect 81 is the method of aspect 76, wherein R80 is O.

Aspect 82 is the method of aspect 76, wherein the compound of formula VI is further defined as

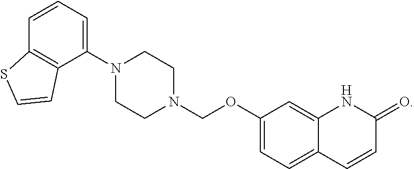 [SMILES: O=c1ccc2ccc(OCN3CCN(c4cccc5sccc45)CC3)cc2[nH]1]

Aspect 83 is the method of any one of aspects 1-41, wherein the NSUN1/2-ACS inhibitor comprises a compound of formula VII:

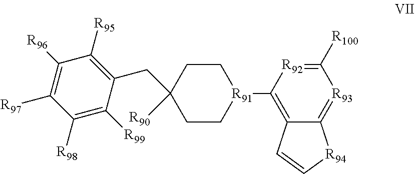

wherein:
   R₉₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₁ is CH or N;
   R₉₂ is CH or N;
   R₉₃ is CH or RN;
   R₉₄ is CH₂, O, S, NH, or N-alkyl;
   R₉₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₀₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 84 is the method of aspect 83, wherein R94 is NH.

Aspect 85 is the method of aspect 83, wherein R91 R92, and R93 are N.

Aspect 86 is the method of aspect 83, wherein R90 is NH2.

Aspect 87 is the method of aspect 83, wherein R97 is a halide.

Aspect 88 is the method of aspect 87, wherein R97 is —Cl.

Aspect 89 is the method of aspect 83, wherein the compound of formula VII is further defined as

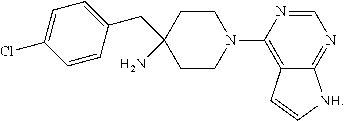 [SMILES: NC1(Cc2ccc(Cl)cc2)CCN(c2ncnc3[nH]ccc23)CC1]

Aspect 90 is the method of any one of aspects 1-41, wherein the NSUN1/2-ACS inhibitor comprises a compound of formula VIII:

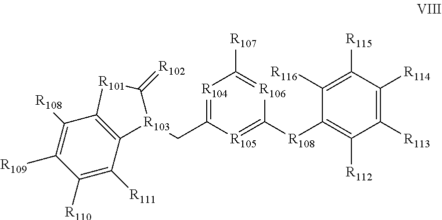

wherein:
   R₁₀₁ is CH₂, NH, S, or O;
   R₁₀₂ is 0 or S;
   R₁₀₃ is CH or N;
   R₁₀₄ is CH, C-alkyl, or N;
   R₁₀₅ is CH, C-alkyl, or N;
   R₁₀₆ is CH, C-alkyl, or N;
   R₁₀₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₀₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₀₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; or an enantiomer, diastereomer, racemic mixture, or salt thereof.

Aspect 91 is the method of aspect 90, wherein R107 is NH2.

Aspect 92 is the method of aspect 90, wherein R104 R105, and R106 are N.

Aspect 93 is the method of aspect 90, wherein R108 is NH.

Aspect 94 is the method of aspect 90, wherein R101 and R102 are O.

Aspect 95 is the method of aspect 90, wherein R103 is N.

Aspect 96 is the method of aspect 90, wherein the compound of formula VIII is further defined as

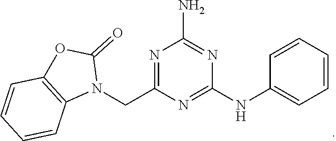 [SMILES: Nc1nc(Cn2c(=O)oc3ccccc32)nc(Nc2ccccc2)n1]

Aspect 97 is the method of any one of aspects 1-41, wherein the NSUN1/2-ACS inhibitor comprises a compound of formula IX:

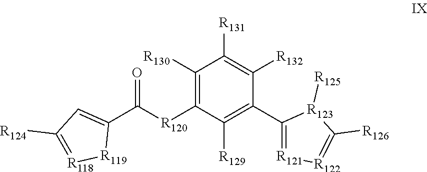

wherein
   R₁₁₈ is N or CH;
   R₁₁₉ is CH₂, NH, O, or S;
   R₁₂₀ is CH₂, or NH;
   R₁₂₁ is N or CH;
   R₁₂₂ is CH or N;
   R₁₂₃ is CH or N; and
   R₁₂₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₂₅ and R₁₂₆ are each independently H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide, or join to form a carbocyclic or heterocyclic ring;
or an enantiomer, diastereomer, racemic mixture, or salt thereof.

Aspect 98 is the method of aspect 97, wherein R118 is N.

Aspect 99 is the method of aspect 97, wherein R121 R122, and R123 are N.

Aspect 100 is the method of aspect 97, wherein R119 and R120 are NH.

Aspect 101 is the method of aspect 97, wherein R124 is phenyl.

Aspect 102 is the method of aspect 97, wherein R125 and R126 come together to form a 6-member carbocyclic or heterocyclic ring.

Aspect 103 is the method of aspect 97, wherein the compound of formula IX is further defined as

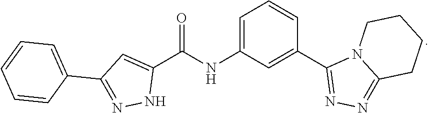 [SMILES: O=C(Nc1cccc(-c2nnc3n2CCCC3)c1)c1cc(-c2ccccc2)n[nH]1]

Aspect 104 is the method of any one of aspects 1-41, wherein the NSUN1/2-ACS inhibitor comprises a compound of formula X:

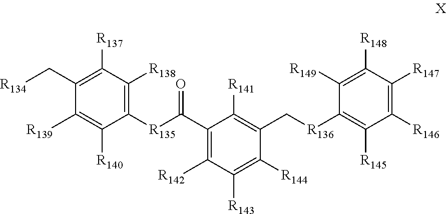

wherein:
   R₁₃₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₃₅ is CH₂ or NH;
   R₁₃₆ is CH₂, NH, O, or S;
   R₁₃₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₃₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₃₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₄₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 105 is the method of aspect 104, wherein R135 is NH.

Aspect 106 is the method of aspect 104, wherein R134 is a 6-member heterocycle.

Aspect 107 is the method of aspect 104, wherein R134 is N-piperidinyl.

Aspect 108 is the method of aspect 104, wherein R147 is a halide.

Aspect 109 is the method of aspect 108, wherein R147 is —Cl.

Aspect 110 is the method of aspect 104, wherein the compound of formula X is further defined as

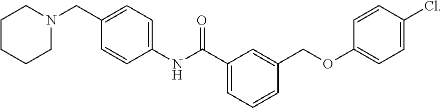 [SMILES: O=C(Nc1ccc(CN2CCCCC2)cc1)c1cccc(COc2ccc(Cl)cc2)c1]

Aspect 111 is the method of any one of aspects 1-41, wherein the NSUN1/2-ACS inhibitor comprises a compound of formula XI:

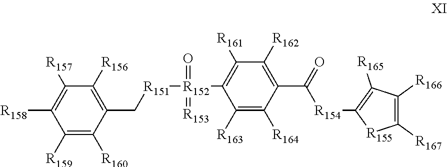

wherein:
   R₁₅₁ is NH or CH₂′
   R₁₅₂ is C or S, with the proviso that when R₁₅₂ is C, R₁₅₃ is nothing, and when R₁₅₂ is S,
   R₁₅₃ is O;
   R₁₅₄ is CH₂ or NH;
   R₁₅₅ is CH₂, NH, O, or S;
   R₁₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₅₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and R₁₆₆ and R₁₆₇ are each independently H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide, or come together to form a substituted or unsubstituted carbocyclic ring or a substituted or unsubstituted heterocyclic ring;
or an enantiomer, diastereomer, racemic mixture, or salt thereof.

Aspect 112 is the method of aspect 111, wherein R₁₆₅ is nitrile.

Aspect 113 is the method of aspect 111, wherein R₁₆₆ and R₁₆₇ come together to form a 6-member carbocyclic ring.

Aspect 114 is the method of aspect 111, wherein R₁₅₂ is S and R₁₅₃ is O.

Aspect 115 is the method of aspect 111, wherein R₁₅₄ is a NH.

Aspect 116 is the method of aspect 111, wherein R₁₅₁ is NH.

Aspect 117 is the method of aspect 111, wherein the compound of formula XI is further defined as

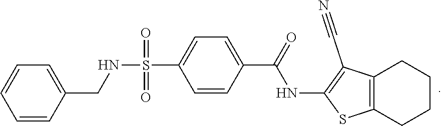 [SMILES: N#Cc1c(NC(=O)c2ccc(S(=O)(=O)NCc3ccccc3)cc2)sc2c1CCCC2]

Aspect 118 is the method of any one of aspects 1-41, wherein the NSUN1/2-ACS inhibitor comprises a compound of formula XII:

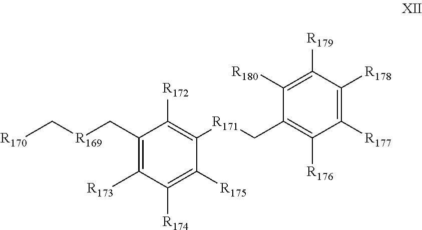

wherein:
   R₁₆₉ is CH₂, NH, O, or S;
   R₁₇₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₁ is CH₂, NH, O, or S;
   R₁₇₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₈₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 119 is the method of aspect 118, wherein R169 is NH.

Aspect 120 is the method of aspect 118, wherein R170 is 4-piperidinyl.

Aspect 121 is the method of aspect 118, wherein R169 is NH.

Aspect 122 is the method of aspect 118, wherein R171 is O.

Aspect 123 is the method of aspect 118, wherein R178 is alkyl.

Aspect 124 is the method of aspect 118, wherein R178 is methyl.

Aspect 125 is the method of aspect 118, wherein R176 is a halide.

Aspect 126 is the method of aspect 118, wherein R176 is —F.

Aspect 127 is the method of aspect 118, wherein the compound of formula XII is further defined as

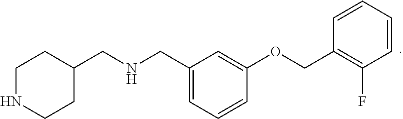 [SMILES: Fc1ccccc1COc1cccc(CNCC2CCNCC2)c1]

Aspect 128 is the method of aspect 118, wherein the compound of formula XII is further defined as

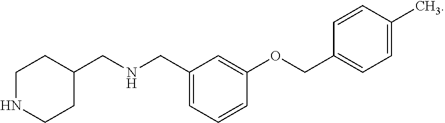 [SMILES: Cc1ccc(COc2cccc(CNCC3CCNCC3)c2)cc1]

Aspect 129 is the method of any one of aspects 1-41, wherein the NSUN1/2-ACS inhibitor comprises a compound of formula XIII:

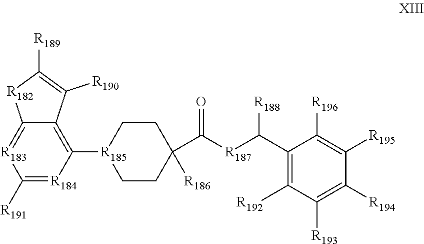

wherein:
   R₁₈₂ is CN₂, NH, O, or S;
   R₁₈₃ is CH or N;
   R₁₈₄ is CH or N;
   R₁₈₅ is CH or N;
   R₁₈₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₈₇ is CH₂ or NH;
   R₁₈₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₈₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₉₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 130 is the method of aspect 129, wherein R183 and R184 are N.

Aspect 131 is the method of aspect 129, wherein R182 is NH.

Aspect 132 is the method of aspect 129, wherein R188 is 2-hydroxyethyl.

Aspect 133 is the method of aspect 129, wherein R187 is NH.

Aspect 134 is the method of aspect 129, wherein R194 is a halide.

Aspect 135 is the method of aspect 129, wherein R194 is —Cl.

Aspect 136 is the method of aspect 129, wherein the compound of formula XIII is further defined as

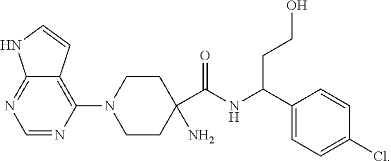 [SMILES: NC1(C(=O)NC(CCO)c2ccc(Cl)cc2)CCN(c2ncnc3[nH]ccc23)CC1]

Aspect 137 is the method of any one of aspects 1-41, wherein the NSUN1/2-ACS inhibitor comprises a compound of formula XIV:

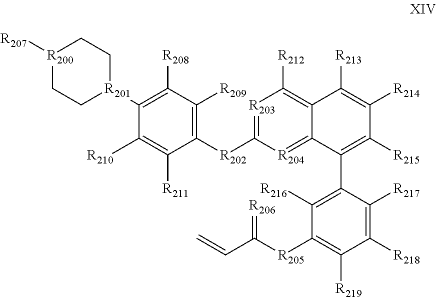

wherein:
   R₂₀₀ is CH or N;
   R₂₀₁ is CH or N;
   R₂₀₂ is CH₂, NH, O, or S;
   R₂₀₃ is CH or N;
   R₂₀₄ is CH or N;
   R₂₀₅ is CH₂ or NH;
   R₂₀₆ is O or R₂₀₆ represents two hydrogen atoms, with each hydrogen atom bound to the geminal carbon atom;
   R₂₀₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₀₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₀₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₂₁₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 137.1 is the method of aspect 137, wherein R₂₀₀ and R₂₀₁ are N.

Aspect 138 is the method of aspect 137, wherein R203 and R204 are N.

Aspect 139 is the method of aspect 137, wherein R207 is 2-hydroxyethyl.

Aspect 140 is the method of aspect 137, wherein R202 is NH.

Aspect 141 is the method of aspect 137, wherein R210 and R211 are each a halide.

Aspect 142 is the method of aspect 137, wherein R210 and R211 are each —F.

Aspect 143 is the method of aspect 137, wherein R205 is NH and R206 is O.

Aspect 144 is the method of aspect 137, wherein the compound of formula XIV is further defined as

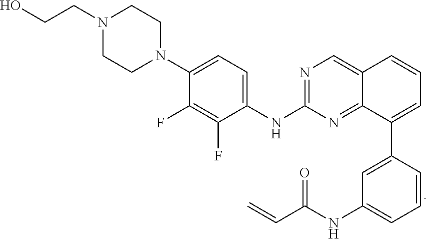 [SMILES: C=CC(=O)Nc1cccc(-c2cccc3cnc(Nc4ccc(N5CCN(CCO)CC5)c(F)c4F)nc23)c1]

Aspect 145 is the method of any one of aspects 1-41, wherein the NSUN1/2-ACS inhibitor comprises a compound of formula XV:

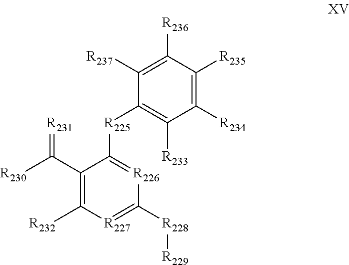

wherein:
   R₂₂₅ is CH₂, NH, O, or S;
   R₂₂₆ is CH or N;
   R₂₂₇ is CH or N;
   R₂₂₈ is CH₂, NH, O, or S;
   R₂₂₉ is is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₁ is O or R₂₃₁ represents two hydrogen atoms, with each hydrogen atom bound to the geminal carbon atom;
   R₂₃₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 146 is the method of aspect 145, wherein R₂₃₄ is alkyl.

Aspect 147 is the method of aspect 145, wherein R234 is methyl.

Aspect 148 is the method of aspect 145, wherein R226 and R227 are N.

Aspect 149 is the method of aspect 145, wherein R228 is NH.

Aspect 150 is the method of aspect 145, wherein R229 is a carbocyclic ring.

Aspect 151 is the method of aspect 145, wherein R229 is a substituted carbocyclic ring.

Aspect 152 is the method of aspect 145, wherein R229 is 2-aminocyclohexyl.

Aspect 153 is the method of aspect 145, wherein the compound of formula XV is further defined as

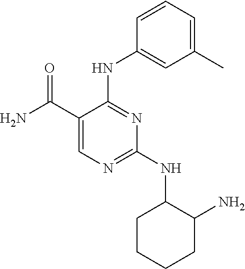 [SMILES: Cc1cccc(Nc2nc(NC3CCCCC3N)ncc2C(N)=O)c1]

Aspect 154 is the method of any one of aspects 1-41, wherein the NSUN1/2-ACS inhibitor comprises a compound of formula XVI:

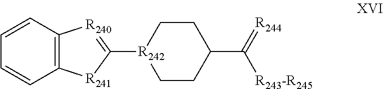

wherein:
   R₂₄₀ is CH or N;
   R₂₄₁ is CH₂, NH, O, or S;
   R₂₄₂ is CH or N;
   R₂₄₃ is CH₂ or NH;
   R₂₄₄ is O or R₂₄₄ represents two hydrogen atoms, with each hydrogen atom bound to the geminal carbon atom; and
   R₂₄₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 155 is the method of aspect 154, wherein R240 and R242 are N.

Aspect 156 is the method of aspect 154, wherein R241 is NH.

Aspect 157 is the method of aspect 154, wherein R243 is NH and R244 is O.

Aspect 158 is the method of aspect 154, wherein R245 is substituted alkyl.

Aspect 159 is the method of aspect 154, wherein R245 is 2-(1-cyclohexenyl)ethyl.

Aspect 160 is the method of aspect 154, wherein the compound of formula XVI is further defined as

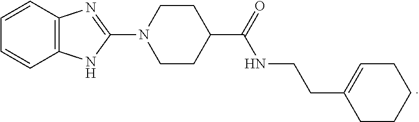 [SMILES: O=C(NCCC1=CCCCC1)C1CCN(c2nc3ccccc3[nH]2)CC1]

Aspect 161 is the method of any one of aspects 1-41, wherein the NSUN1/2-ACS inhibitor comprises a compound of formula XVII:

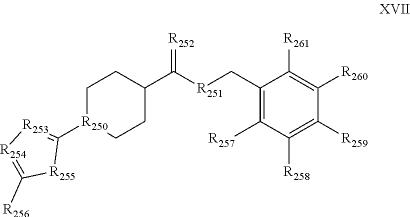

R₂₅₀ is CH or N;
R₂₅₁ is CH₂ or NH;
R₂₅₂ is O or R₂₅₂ represents two hydrogen atoms, with each hydrogen atom bound to the geminal carbon atom;
R₂₅₃ is CH or N;
R₂₅₄ is CH or N;
R₂₅₅ is CH₂, NH, O, or S;
R₂₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₅₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₆₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
R₂₆₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   or an enantiomer, diastereomer, racemic mixture, or salt thereof.

Aspect 162 is the method of aspect 161, wherein R253 and R253 are N.

Aspect 163 is the method of aspect 161, wherein R251 is NH and R252 is O.

Aspect 164 is the method of aspect 161, wherein R255 is S.

Aspect 165 is the method of aspect 161, wherein R255 is O.

Aspect 166 is the method of aspect 161, wherein R256 is phenyl.

Aspect 167 is the method of aspect 161, wherein R256 is N-pyrrole.

Aspect 168 is the method of aspect 161, wherein the compound of formula XVII is further defined as

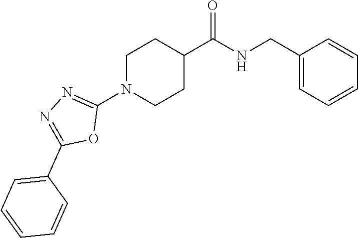 [SMILES: O=C(NCc1ccccc1)C1CCN(c2nnc(-c3ccccc3)o2)CC1]

Aspect 169 is the method of aspect 161, wherein the compound of formula XVII is further defined as

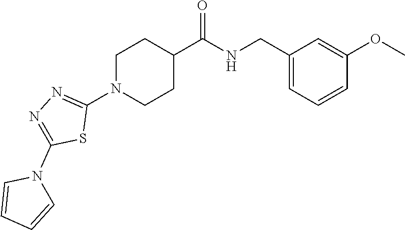 [SMILES: COc1cccc(CNC(=O)C2CCN(c3nnc(-n4cccc4)s3)CC2)c1]

Aspect 170 is the method of any one of aspects 1-41, wherein the NSUN1/2-ACS inhibitor comprises a compound of formula XVIII:

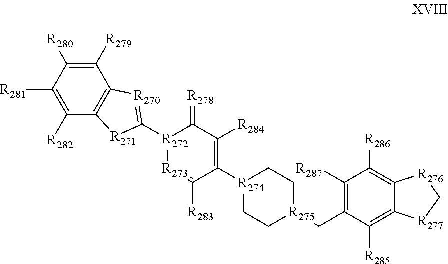

wherein:
   R₂₇₀ is CH or N;
   R₂₇₁ is CH₂, NH, O, or S;
   R₂₇₂ is CH or N;
   R₂₇₃ is CH or N;
   R₂₇₄ is CH or N;
   R₂₇₅ is CH or N;
   R₂₇₆ is CH₂, NH, or O;
   R₂₇₇ is CH₂, NH, or O;
   R₂₇₈ is O or R₂₇₈ represents two hydrogen atoms, with each hydrogen atom bound to the geminal carbon atom;
   R₂₇₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₂₈₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 171 is the method of aspect 170, wherein R276 and R277 are O.

Aspect 172 is the method of aspect 170, wherein R274 and R275 are N.

Aspect 173 is the method of aspect 170, wherein R272 and R273 are N.

Aspect 174 is the method of aspect 170, wherein R270 is N.

Aspect 175 is the method of aspect 170, wherein R271 is NH.

Aspect 176 is the method of aspect 170, wherein R284 is a halide.

Aspect 177 is the method of aspect 170, wherein R284 is —Cl.

Aspect 178 is the method of aspect 161, wherein the compound of formula XVII is further defined as

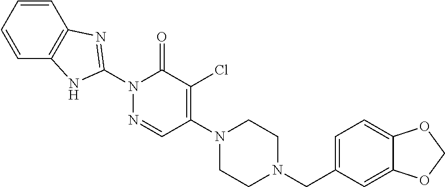 [SMILES: O=c1c(Cl)c(N2CCN(Cc3ccc4c(c3)OCO4)CC2)cnn1-c1nc2ccccc2[nH]1]

Aspect 179 is the method of any one of aspects 1-41, wherein the NSUN1/2-ACS inhibitor comprises a compound of formula XIX:

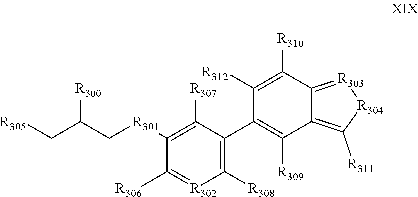

wherein:
   R₃₀₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₁ is CH₂, NH, O, or S;
   R₃₀₂ is CH or N;
   R₃₀₃ is CH or N;
   R₃₀₄ is CH₂, NH, O, or S;
   R₃₀₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₃₁₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 180 is the method of aspect 179, wherein R302 is N.

Aspect 181 is the method of aspect 179, wherein R300 is NH2.

Aspect 182 is the method of aspect 179, wherein R301 is O.

Aspect 183 is the method of aspect 179, wherein R305 is phenyl.

Aspect 184 is the method of aspect 179, wherein R305 is 3-indole.

Aspect 185 is the method of aspect 179, wherein R311 is alkyl.

Aspect 186 is the method of aspect 179, wherein R311 is methyl.

Aspect 187 is the method of aspect 179, wherein the compound of formula XVII is further defined as

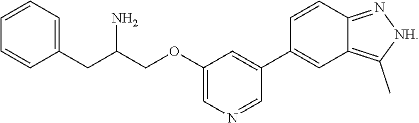 [SMILES: Cc1[nH]nc2ccc(-c3cncc(OCC(N)Cc4ccccc4)c3)cc12]

Aspect 188 is the method of aspect 179, wherein the compound of formula XVII is further defined as

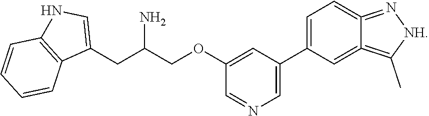

Aspect 189 a method of overcoming and/or preventing cancer cell drug resistance to venetoclax-based and/or 5-azacytidine-based therapies comprising, contacting the cancer cell with one or more inhibitors of NSUN1 and/or NSUN2 interactions with Active Chromatin Site (ACS) associated proteins and/or complexes (NSUN1/2-ACS inhibitor).

Aspect 190 is the method of aspect 189, wherein overcoming and/or preventing cancer cell drug resistance comprises administration of any one or more NSUN1/2-ACS inhibitors described in Tables 2-6.

Aspect 191 a method of treating refractory cancer comprising, treatment with an NSUN1 and/or NSUN2 protein-protein interaction inhibitor with a NSUN1/2-ACS inhibitor.

Aspect 192 is the method of aspect 191, wherein treating comprises administration of any one or more NSUN1/2-ACS inhibitors described in Tables 2-6 and/or a derivative thereof, and/or optionally wherein the method does not comprise administration of any one or more of NSUN1-38, NSUN1-33, NSUN1-36, NSUN1-28, NSUN1-4, NSUN1-32, NSUN1-43, NSUN1-44, NSUN1-46, NSUN1-41, NSUN2-43, NSUN2-51, NSUN2-41, NSUN2-29, NSUN2-23, NSUN2-55, NSUN2-22, NSUN2-26, NSUN2-2, NSUN2-52, NSUN2-49, NSUN2-56, NSUN2-66, NSUN2-40, and/or NSUN2-95.

Aspect 193 a composition comprising NSUN2-11, NSUN2-37, NSUN1-9, NSUN1-10, NSUN1-11, NSUN1-17, NSUN1-17.13, NSUN1-17.16, NSUN1-24, NSUN1-25, NSUN1-27, NSUN1-30, NSUN1-31, NSUN1-54, NSUN1-64, NSUN1-67, NSUN1-73, NSUN1-74, NSUN2-3, NSUN2-10, NSUN2-17, NSUN2-18, NSUN2-19, NSUN2-20, NSUN2-21, NSUN2-35, NSUN2-38, NSUN2-39, NSUN2-64, NSUN2-98, and/or a derivative thereof, and a pharmaceutically acceptable excipient.

Aspect 194 is the composition of aspect 193, wherein the composition comprises a derivative of compound NSUN1-9, NSUN1-10, NSUN1-11, NSUN1-17, NSUN1-17.13, NSUN1-17.16, NSUN1-24, NSUN1-25, NSUN1-27, NSUN1-30, NSUN1-31, NSUN1-54, NSUN1-64, NSUN1-67, NSUN1-73, NSUN1-74, NSUN2-3, NSUN2-10, NSUN2-11, NSUN2-17, NSUN2-18, NSUN2-19, NSUN2-20, NSUN2-21, NSUN2-35, NSUN2-37, NSUN2-38, NSUN2-39, NSUN2-64, and/or NSUN2-98.

Aspect 195 is the composition of aspect 194, wherein the derivative is a water soluble derivative.

Aspect 196 is the composition of aspect 193, comprising NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-11, NSUN2-37, NSUN2-35, and/or a derivative thereof.

Aspect 197 is the composition of aspect 196, wherein the composition comprises a derivative of compound NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-11, NSUN2-37, NSUN2-35, and/or a derivative thereof.

Aspect 198 is the composition of aspect 197, wherein the derivative is a water soluble derivative.

Aspect 199 is the composition of aspect 193, comprising NSUN1-17.16, NSUN1-11, NSUN1-67, NSUN2-11, NSUN2-37, NSUN2-35, and/or a derivative thereof.

Aspect 200 is the composition of aspect 199, wherein the composition comprises a derivative of compound NSUN2-11, NSUN2-37, NSUN1-17.16, NSUN1-11, NSUN1-67, NSUN2-37, and/or NSUN2-35.

Aspect 201 is the composition of aspect 200, wherein the derivative is a water soluble derivative.

Aspect 202 is the composition of any one of aspects 193-201, comprising, consists essentially of, or consisting of NSUN1-11 and/or a derivative thereof.

Aspect 203 is the composition of any one of aspects 193-201, comprising, consists essentially of, or consisting of NSUN1-67 and/or a derivative thereof.

Aspect 204 is the composition of any one of aspects 193-201, comprising, consists essentially of, or consisting of NSUN1-17.16 and/or a derivative thereof.

Aspect 205 is the composition of any one of aspects 193-201, comprising, consists essentially of, or consisting of NSUN2-11 and/or a derivative thereof.

Aspect 206 is the composition of any one of aspects 193-201, comprising, consists essentially of, or consisting of NSUN2-37 and/or a derivative thereof.

Aspect 207 is the composition of any one of aspects 193-201, comprising, consists essentially of, or consisting of NSUN2-35 and/or a derivative thereof.

Aspect 208 is the composition of any one of aspects 193-207, further comprising venetoclax and/or 5-azacytidine.

Aspect 209 a composition comprising a compound of formula I

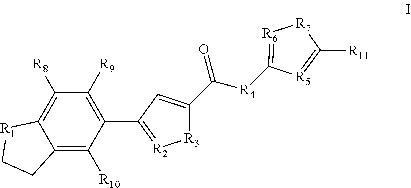

wherein:
   R₁ is CH₂, NH, O, or S;
   R₂ is CH or N;
   R₃ is CH₂, NH, O, or S;
   R₄ is NH or CH₂;
   R₅ is N or CH;
   R₆ is N or CH;
   R₇ is CH₂, NH, O, or S;
   R₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; or an enantiomer, diastereomer, racemic mixture, or salt thereof.

Aspect 210 is the composition of aspect 209, wherein R1 is O.

Aspect 211 is the composition of aspect 209, wherein R4 is NH.

Aspect 212 is the composition of aspect 209, wherein R11 is alkyl.

Aspect 213 is the composition of aspect 209, wherein R11 is isopropyl.

Aspect 214 is the composition of aspect 209, wherein R3 and R7 are NH.

Aspect 215 is the composition of aspect 209, wherein R2, R5, and R6 are N.

Aspect 216 is the composition of aspect 209, wherein the compound of formula I is further defined as

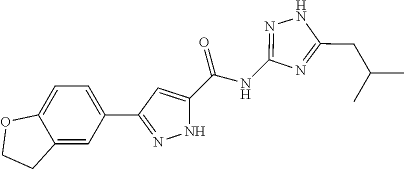 [SMILES: CC(C)Cc1nc(NC(=O)c2cc(-c3ccc4c(c3)CCO4)n[nH]2)n[nH]1]

Aspect 217 a composition comprising a compound of formula II:

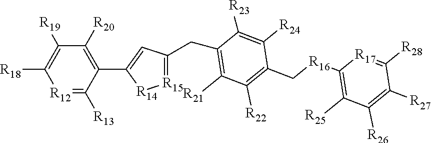

wherein:
   R₁₂ is CH or N;
   R₁₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄ is CH₂, NH, O, or S;
   R₁₅ is CH or N;
   R₁₆ is CH₂, NH, O, or S;
   R₁₇ is CH or N;
   R₁₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 218 is the composition of aspect 217, wherein R14 and R16 are O.

Aspect 219 is the composition of aspect 217, wherein R12, R15, and R17 are N.

Aspect 220 is the composition of aspect 217, wherein R12 is NH2.

Aspect 221 a composition comprising a compound of formula III:

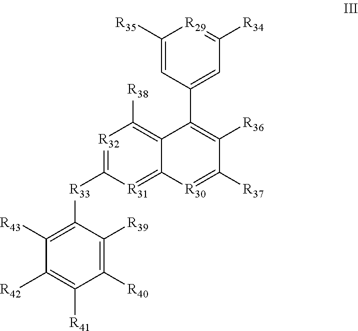

wherein:
   R₂₉ is N or CH;
   R₃₀ is N or CH;
   R₃₁ is N or CH;
   R₃₂ is N or CH;
   R₃₃ is CH₂, NH, O, or S;
   R₃₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₄₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₄₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₄₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₄₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 222 is the composition of aspect 221, wherein R30, R31, R32, and R33 are N.

Aspect 223 is the composition of aspect 221, wherein R38 is OH.

Aspect 224 is the composition of aspect 221, wherein R41 is a halide.

Aspect 225 is the composition of aspect 221, wherein R41 is —Cl.

Aspect 226 is the composition of aspect 221, wherein R33 is NH.

Aspect 227 is the composition of aspect 221, wherein the compound of formula III is further defined as

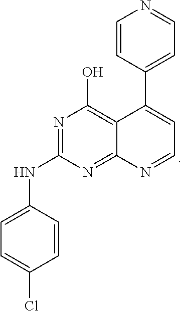 [SMILES: Oc1nc(Nc2ccc(Cl)cc2)nc2nccc(-c3ccncc3)c12]

Aspect 228 a composition comprising a compound of formula IV:

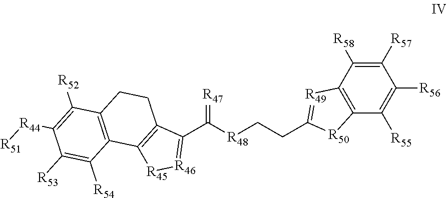

wherein:
   R₄₄ is O, S, NH, or CH₂
   R₄₅ is CH₂, NH, O, or S
   R₄₆ is CH or N;
   R₄₇ is NH, N-alkyl, O, or S;
   R₄₈ is CH₂ or NH;
   R₄₉ is CH or N;
   R₅₀ is CH₂, NH, O, or S;
   R₅₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 229 is the composition of aspect 228, wherein R45, R48, and R50 are NH.

Aspect 230 is the composition of aspect 228, wherein R44 and R47 are O.

Aspect 231 is the composition of aspect 228, wherein R51 and R57 are alkyl.

Aspect 232 is the composition of aspect 228, wherein R51 is methyl.

Aspect 233 is the composition of aspect 228, wherein R57 is methyl.

Aspect 234 is the composition of aspect 228, wherein the compound of formula IV is further defined as

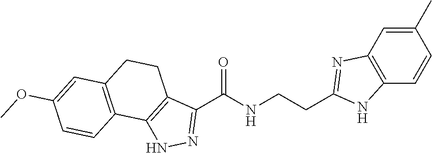

Aspect 235 a composition comprising a compound of formula V:

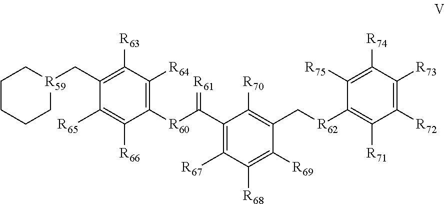

wherein:
R₅₉ is CH or N;
R₆₀ is CN₂, NH, O, or S;
R₆₁ is O or S;
R₆₂ is CH₂, NH, O, or S;
R₆₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 236 is the composition of aspect 235, wherein R59 is N.

Aspect 237 is the composition of aspect 235, wherein R60 is NH.

Aspect 238 is the composition of aspect 235, wherein R51 is a halogen.

Aspect 239 is the composition of aspect 235, wherein R51 is —Br.

Aspect 240 is the composition of aspect 235, wherein R62 is O.

Aspect 241 is the composition of aspect 235, wherein the compound of formula V is further defined as

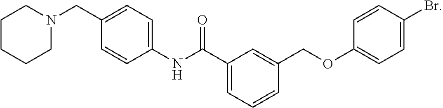 [SMILES: O=C(Nc1ccc(CN2CCCCC2)cc1)c1cccc(COc2ccc(Br)cc2)c1]

Aspect 242 a composition comprising a compound of formula VI:

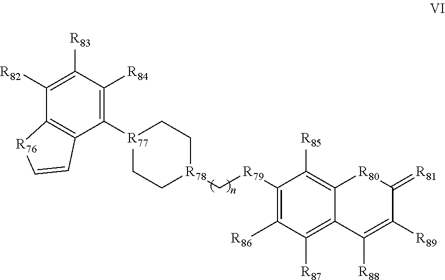

wherein:
   R₇₆ is CH₂, NH, O, or S;
   R₇₇ is CH or N;
   R₇₈ is CH or N;
   R₇₉ is CH₂, NH, S, or O;
   R₈₀ is CH₂, NH, O, or S;
   R₈₁ is CH₂, NH, or O;
   R₈₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₈₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₈₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 243 is the composition of aspect 242, wherein R76 is S.

Aspect 244 is the composition of aspect 242, wherein R77 and R78 are N.

Aspect 245 is the composition of aspect 242, wherein R80 is NH.

Aspect 246 is the composition of aspect 242, wherein R79 is O.

Aspect 247 is the composition of aspect 242, wherein R80 is O.

Aspect 248 a composition comprising a compound of formula VII:

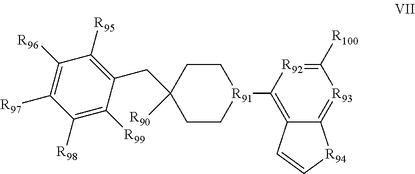

wherein:
   R₉₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₁ is CH or N;
   R₉₂ is CH or N;
   R₉₃ is CH or N;
   R₉₄ is CH₂, O, S, NH, or N-alkyl;
   R₉₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₉₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₀₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 249 is the composition of aspect 248, wherein R94 is NH.

Aspect 250 is the composition of aspect 248, wherein R91 R92, and R93 are N.

Aspect 251 is the composition of aspect 248, wherein R90 is NH2.

Aspect 252 is the composition of aspect 248, wherein R97 is a halide.

Aspect 253 is the composition of aspect 248, wherein R97 is —Cl.

Aspect 254 is the composition of aspect 248, wherein the compound of formula VII is further defined as

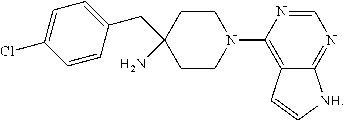 [SMILES: NC1(Cc2ccc(Cl)cc2)CCN(c2ncnc3[nH]ccc23)CC1]

Aspect 255 a composition comprising a compound of formula VIII:

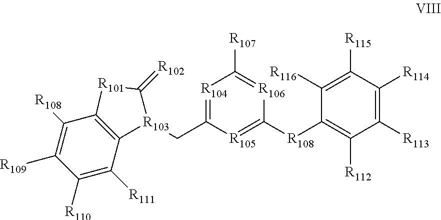

wherein
   R₁₀₁ is CH₂, NH, S, or O;
   R₁₀₂ is O or S;
   R₁₀₃ is CH or N;
   R₁₀₄ is CH, C-alkyl, or N;
   R₁₀₅ is CH, C-alkyl, or N;
   R₁₀₆ is CH, C-alkyl, or N;
   R₁₀₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₀₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₀₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₁₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; or an enantiomer, diastereomer, racemic mixture, or salt thereof.

Aspect 256 is the composition of aspect 255, wherein R107 is NH2.

Aspect 257 is the composition of aspect 255, wherein R104 R105, and R106 are N.

Aspect 258 is the composition of aspect 255, wherein R108 is NH.

Aspect 259 is the composition of aspect 255, wherein R101 and R102 are O.

Aspect 260 is the composition of aspect 255, wherein R103 is N.

Aspect 261 is the composition of aspect 255, wherein the compound of formula VIII is further defined as

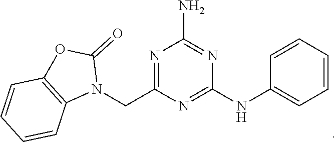 [SMILES: Nc1nc(Cn2c(=O)oc3ccccc32)nc(Nc2ccccc2)n1]

Aspect 262 a composition comprising a compound of formula IX:

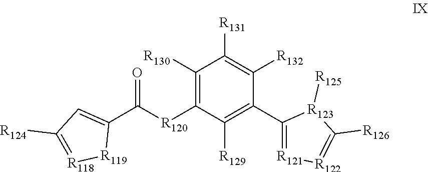

wherein
   R₁₁₈ is N or CH;
   R₁₁₉ is CH₂, NH, O, or S;
   R₁₂₀ is CH₂, or NH;
   R₁₂₁ is N or CH;
   R₁₂₂ is CH or N;
   R₁₂₃ is CH or N; and
   R₁₂₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₂₅ and R₁₂₆ are each independently H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide, or join to form a carbocyclic or heterocyclic ring;
or an enantiomer, diastereomer, racemic mixture, or salt thereof.

Aspect 263 is the composition of aspect 262, wherein R118 is N.

Aspect 264 is the composition of aspect 262, wherein R121 R122, and R123 are N.

Aspect 265 is the composition of aspect 262, wherein R119 and R120 are NH.

Aspect 266 is the composition of aspect 262, wherein R124 is phenyl.

Aspect 267 is the composition of aspect 262, wherein R125 and R126 come together to form a 6-member carbocyclic or heterocyclic ring.

Aspect 268 is the composition of aspect 262, wherein the compound of formula IX is further defined as

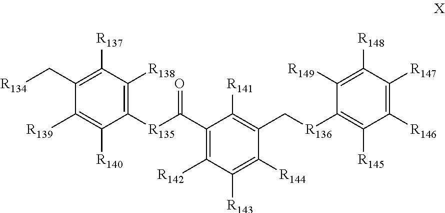

Aspect 269 a composition comprising a compound of formula X:

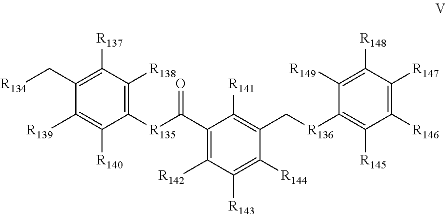

wherein:
   R₁₃₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₃₅ is CH₂ or NH;
   R₁₃₆ is CH₂, NH, O, or S;
   R₁₃₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₃₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₃₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₄₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₄₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 270 is the composition of aspect 269, wherein R135 is NH.

Aspect 271 is the composition of aspect 269, wherein R134 is a 6-member heterocycle.

Aspect 272 is the composition of aspect 269, wherein R134 is N-piperidinyl.

Aspect 273 is the composition of aspect 269, wherein R147 is a halide.

Aspect 274 is the composition of aspect 269, wherein R147 is —Cl.

Aspect 275 is the composition of aspect 269, wherein the compound of formula X is further defined as

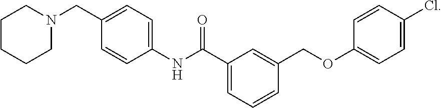 [SMILES: O=C(Nc1ccc(CN2CCCCC2)cc1)c1cccc(COc2ccc(Cl)cc2)c1]

Aspect 276 a composition comprising a compound of formula XI.

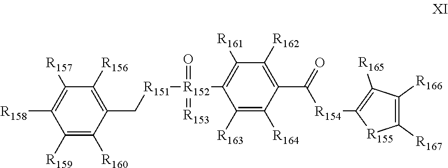

wherein:
   R₁₅₁ is NH or CH₂′
   R₁₅₂ is C or S, with the proviso that when R₁₅₂ is C, R₁₅₃ is nothing, and when R₁₅₂ is S,
   R₁₅₃ is O;
   R₁₅₄ is CH₂ or NH;
   R₁₅₅ is CH₂, NH, O, or S;
   R₁₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₅₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₆₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₆₆ and R₁₆₇ are each independently H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide, or come together to form a substituted or unsubstituted carbocyclic ring or a substituted or unsubstituted heterocyclic ring;
or an enantiomer, diastereomer, racemic mixture, or salt thereof.

Aspect 277 is the composition of aspect 276, wherein R165 is nitrile.

Aspect 278 is the composition of aspect 276, wherein R166 and R167 come together to form a 6-member carbocyclic ring.

Aspect 279 is the composition of aspect 276, wherein R152 is S and R153 is O.

Aspect 280 is the composition of aspect 276, wherein R154 is a NH.

Aspect 281 is the composition of aspect 276, wherein R151 is NH.

Aspect 282 is the composition of aspect 276, wherein the compound of formula XI is further defined as

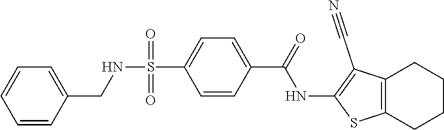 [SMILES: N#Cc1c(NC(=O)c2ccc(S(=O)(=O)NCc3ccccc3)cc2)sc2c1CCCC2]

Aspect 283 a composition comprising a compound of formula XII.

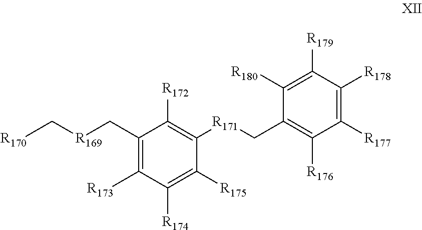

wherein:
   R₁₆₉ is CH₂, NH, O, or S;
   R₁₇₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₁ is CH₂, NH, O, or S;
   R₁₇₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₇₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₈₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 284 is the composition of aspect 283, wherein R169 is NH.

Aspect 285 is the composition of aspect 283, wherein R170 is 4-piperidinyl.

Aspect 286 is the composition of aspect 283, wherein R169 is NH.

Aspect 287 is the composition of aspect 283, wherein R171 is O.

Aspect 288 is the composition of aspect 283, wherein R178 is alkyl.

Aspect 289 is the composition of aspect 283, wherein R178 is methyl.

Aspect 290 is the composition of aspect 283, wherein R176 is a halide.

Aspect 291 is the composition of aspect 283, wherein R176 is —F.

Aspect 292 is the composition of aspect 283, wherein the compound of formula XII is further defined as

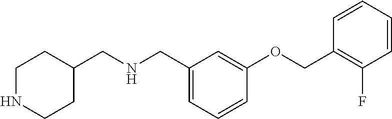 [SMILES: Fc1ccccc1COc1cccc(CNCC2CCNCC2)c1]

Aspect 293 is the composition of aspect 283, wherein the compound of formula XII is further defined as

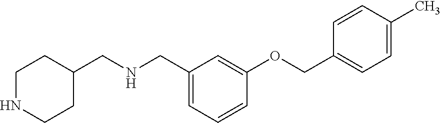 [SMILES: Cc1ccc(COc2cccc(CNCC3CCNCC3)c2)cc1]

Aspect 294 a composition comprising a compound of formula XIII:

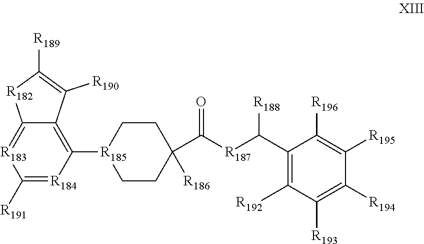

wherein:
   R₁₈₂ is CN₂, NH, O, or S;
   R₁₈₃ is CH or N;
   R₁₈₄ is C H or N;
   R₁₈₅ is CH or N;
   R₁₈₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₈₇ is CH₂ or NH;
   R₁₈₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₈₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₁₉₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₁₉₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 295 is the composition of aspect 294, wherein R183 and R184 are N.

Aspect 296 is the composition of aspect 294, wherein R182 is NH.

Aspect 297 is the composition of aspect 294, wherein R188 is 2-hydroxyethyl.

Aspect 298 is the composition of aspect 294, wherein R187 is NH.

Aspect 299 is the composition of aspect 294, wherein R194 is a halide.

Aspect 300 is the composition of aspect 294, wherein R194 is —Cl.

Aspect 301 a composition comprising a compound of formula XIV:

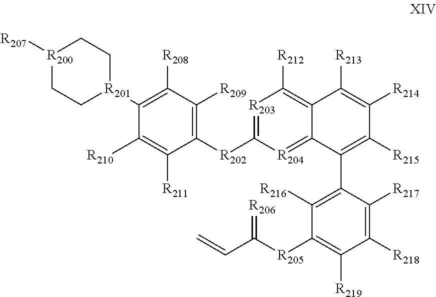

wherein:
   R₂₀₀ is CH or N;
   R₂₀₁ is CH or N;
   R₂₀₂ is CH₂, NH, O, or S;
   R₂₀₃ is CH or N;
   R₂₀₄ is CH or N;
   R₂₀₅ is CH₂ or NH;
   R₂₀₆ is O or R₂₀₆ represents two hydrogen atoms, with each hydrogen atom bound to the geminal carbon atom;
   R₂₀₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₀₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₀₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₁₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₂₁₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;

Aspect 301.1 is the composition of aspect 301, wherein R200 and R201 are N.

Aspect 302 is the composition of aspect 301, wherein R203 and R204 are N.

Aspect 303 is the composition of aspect 301, wherein R207 is 2-hydroxyethyl.

Aspect 304 is the composition of aspect 301, wherein R202 is NH.

Aspect 305 is the composition of aspect 301, wherein R210 and R211 are each a halide.

Aspect 306 is the composition of aspect 301, wherein R210 and R211 are each —F.

Aspect 307 is the composition of aspect 301, wherein R205 is NH and R206 is O.

Aspect 308 is the composition of aspect 301, wherein the compound of formula XIV is further defined as

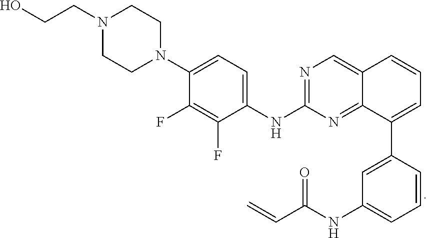 [SMILES: C=CC(=O)Nc1cccc(-c2cccc3cnc(Nc4ccc(N5CCN(CCO)CC5)c(F)c4F)nc23)c1]

Aspect 309 a composition comprising a compound of formula XV:

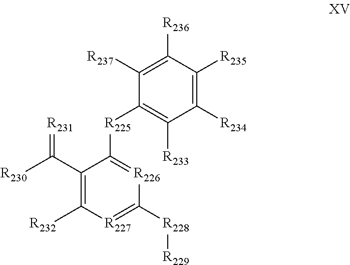

wherein:
   R₂₂₅ is CH₂, NH, O, or S;
   R₂₂₆ is CH or N;
   R₂₂₇ is CH or N;
   R₂₂₈ is CH₂, NH, O, or S;
   R₂₂₉ is is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₁ is O or R₂₃₁ represents two hydrogen atoms, with each hydrogen atom bound to the geminal carbon atom;
   R₂₃₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₃₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 310 is the composition of aspect 309, wherein R234 is alkyl.

Aspect 311 is the composition of aspect 309, wherein R234 is methyl.

Aspect 312 is the composition of aspect 309, wherein R226 and R227 are N.

Aspect 313 is the composition of aspect 309, wherein R228 is NH.

Aspect 314 is the composition of aspect 309, wherein R229 is a carbocyclic ring.

Aspect 315 is the composition of aspect 309, wherein R229 is a substituted carbocyclic ring.

Aspect 316 is the composition of aspect 309, wherein R229 is 2-aminocyclohexyl.

Aspect 317 is the composition of aspect 309, wherein the compound of formula XV is further defined as

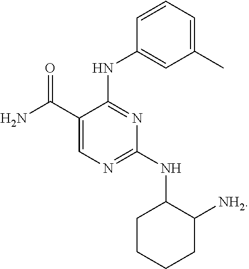 [SMILES: Cc1cccc(Nc2nc(NC3CCCCC3N)ncc2C(N)=O)c1]

Aspect 318 is a composition comprising a compound of formula XVI:

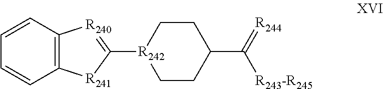

wherein:
   R₂₄₀ is CH or N;
   R₂₄₁ is CH₂, NH, O, or S;
   R₂₄₂ is CH or N;
   R₂₄₃ is CH₂ or NH;
   R₂₄₄ is O or R₂₄₄ represents two hydrogen atoms, with each hydrogen atom bound to the geminal carbon atom; and
   R₂₄₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; or an enantiomer, diastereomer, racemic mixture, or salt thereof.

Aspect 319 is the composition of aspect 318, wherein R240 and R242 are N.

Aspect 320 is the composition of aspect 318, wherein R241 is NH.

Aspect 321 is the composition of aspect 318, wherein R243 is NH and R244 is O.

Aspect 322 is the composition of aspect 318, wherein R245 is substituted alkyl.

Aspect 323 is the composition of aspect 318, wherein R245 is 2-(1-cyclohexenyl)ethyl.

Aspect 324 is the composition of aspect 318, wherein the compound of formula XVI is further defined as

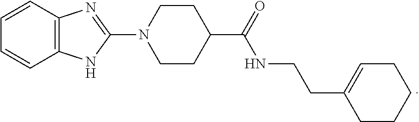 [SMILES: O=C(NCCC1=CCCCC1)C1CCN(c2nc3ccccc3[nH]2)CC1]

Aspect 325 a composition comprising a compound of formula XVII.

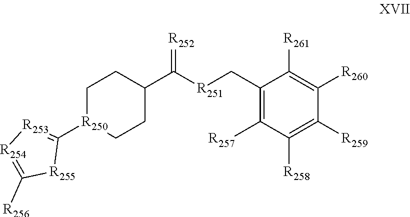

R₂₅₀ is CH₂ or N;
R₂₅₂ is O or R₂₅₂ represents two hydrogen atoms, with each hydrogen atom bound to the geminal carbon atom;
R₂₅₃ is CH or N;
R₂₅₄ is CH or N;
R₂₅₅ is CH₂, NH, O, or S;
R₂₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₅₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₂₆₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
R₂₆₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   or an enantiomer, diastereomer, racemic mixture, or salt thereof.

Aspect 326 is the composition of aspect 325, wherein R253 and R253 are N.

Aspect 327 is the composition of aspect 325, wherein R251 is NH and R252 is O.

Aspect 328 is the composition of aspect 325, wherein R255 is S.

Aspect 329 is the composition of aspect 325, wherein R255 is O.

Aspect 330 is the composition of aspect 325, wherein R256 is phenyl.

Aspect 331 is the composition of aspect 325, wherein R256 is N-pyrrole.

Aspect 332 is the composition of aspect 325, wherein the compound of formula XVII is further defined as

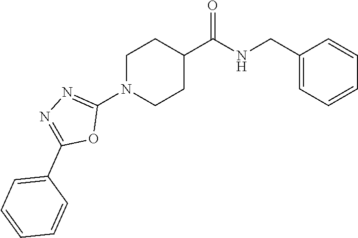 [SMILES: O=C(NCc1ccccc1)C1CCN(c2nnc(-c3ccccc3)o2)CC1]

Aspect 333 is the composition of aspect 325, wherein the compound of formula XVII is further defined as

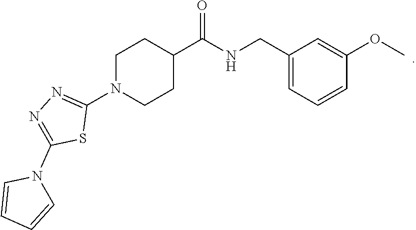 [SMILES: COc1cccc(CNC(=O)C2CCN(c3nnc(-n4cccc4)s3)CC2)c1]

Aspect 334 a composition comprising a compound of formula XVIII:

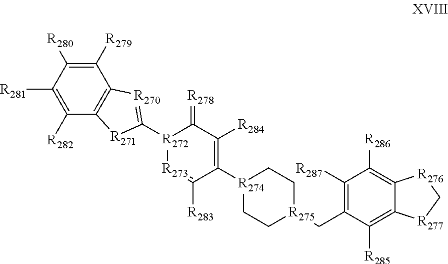

wherein:
   R₂₇₀ is CH or N;
   R₂₇₁ is CH₂, NH, O, or S;
   R₂₇₂ is CH or N;
   R₂₇₃ is CH or N;
   R₂₇₄ is CH or N;
   R₂₇₅ is CH or N;
   R₂₇₆ is CH₂, NH, or O;
   R₂₇₇ is CH₂, NH, or O;
   R₂₇₈ is O or R₂₇₈ represents two hydrogen atoms, with each hydrogen atom bound to the geminal carbon atom;
   R₂₇₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₂₈₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₂₈₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 335 is the composition of aspect 334, wherein R276 and R277 are O.

Aspect 336 is the composition of aspect 334, wherein R274 and R275 are N.

Aspect 337 is the composition of aspect 334, wherein R272 and R273 are N.

Aspect 338 is the composition of aspect 334, wherein R270 is N.

Aspect 339 is the composition of aspect 334, wherein R271 is NH.

Aspect 340 is the composition of aspect 334, wherein R284 is a halide.

Aspect 341 is the composition of aspect 334, wherein R284 is —Cl.

Aspect 342 is the composition of aspect 334, wherein the compound of formula XVII is further defined as

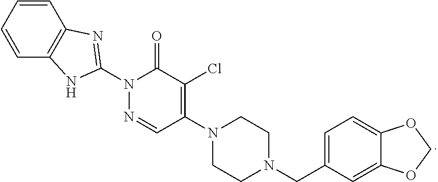 [SMILES: O=c1c(Cl)c(N2CCN(Cc3ccc4c(c3)OCO4)CC2)cnn1-c1nc2ccccc2[nH]1]

Aspect 343 a composition comprising a compound of formula XIX:

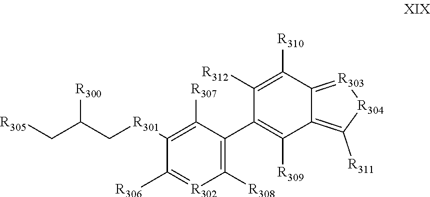

wherein:
   R₃₀₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₁ is CH₂, NH, O, or S;
   R₃₀₂ is CH or N;
   R₃₀₃ is CH or N;
   R₃₀₄ is CH₂, NH, O, or S;
   R₃₀₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₀₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
   R₃₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
   R₃₁₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

Aspect 344 is the composition of aspect 343, wherein R302 is N.

Aspect 345 is the composition of aspect 343, wherein R300 is NH2.

Aspect 346 is the composition of aspect 343, wherein R301 is O.

Aspect 347 is the composition of aspect 343, wherein R305 is phenyl.

Aspect 348 is the composition of aspect 343, wherein R305 is 3-indole.

Aspect 349 is the composition of aspect 343, wherein R311 is alkyl.

Aspect 350 is the composition of aspect 343, wherein R311 is methyl.

Aspect 351 is the composition of aspect 334, wherein the compound of formula XVII is further defined as

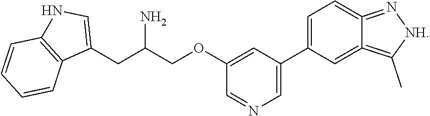

### Examples of Definitions

In keeping with long-standing patent law convention, the words “a” and “an” when used in the present specification in concert with the word comprising, including the claims, denote “one or more.” Some aspects of the disclosure may consist of or consist essentially of one or more elements, method steps, and/or methods of the disclosure. It is contemplated that any method or composition described herein can be implemented with respect to any other method or composition described herein and that different aspects may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

Throughout this application, the term “about” is used according to its plain and ordinary meaning in the area of cell and molecular biology to indicate that a value includes the standard deviation of error for the device or method being employed to determine the value.

As used herein, the terms “individual,” “subject,” and “patient” are used interchangeably and can refer to a human or non-human.

As used herein, the term “nitro” means —NO₂; the term “amino” means —NH₂; the term “halo” or “halogen” designates —F, —Cl, —Br or —I; the term “mercapto” means —SH; the terms “cyano” and “nitrile” mean —CN; the term “azido” means —N₃; the term “silyl” means —SiH₃, and the term “hydroxy” means —OH.

The term “alkyl,” by itself or as part of another substituent, means, unless otherwise stated, a linear (i.e., unbranched) or branched carbon chain of 1, 2, 3, 4, 5, 6, 7, 8, 9, or 10 carbons, which may be fully saturated, monounsaturated, or polyunsaturated. An unsaturated alkyl group includes those having one or more carbon-carbon double bonds (alkenyl) and those having one or more carbon-carbon triple bonds (alkynyl). The groups, —CH₃ (Me, methyl), —CH₂CH₃ (Et, ethyl), —CH₂CH₂CH₃ (n-Pr, n-propyl), propenyl, —CH(CH₃)₂(iso-Pr, iso-propyl), —CH₂CH₂CH₂CH₃ (n-Bu, n-butyl), butenyl, —CH(CH₃)CH₂CH₃ (sec-butyl), —CH₂CH(CH₃)₂(iso-butyl), —C(CH₃)₃ (tert-butyl), —CH₂C(CH₃)₃ (neo-pentyl), propargyl, are all non-limiting examples of alkyl groups. Specifically included within the definition of “alkyl” are those alkyl groups that are optionally substituted.

The term “heteroalkyl,” by itself or in combination with another term, means, unless otherwise stated, a linear or branched chain having at least one carbon atom and at least one heteroatom selected from the group consisting of O, N, S, P, and Si. The heteroatom(s) may be placed at any interior position of the heteroalkyl group or at the position at which the heteroalkyl group is attached to the remainder of the molecule. Up to two heteroatoms may be consecutive. The following groups are all non-limiting examples of heteroalkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂SCH₃, —CH₂CH₂OH, —CH₂CH₂OC(O)CH₃, —CH₂CH₂NHCO₂C(CH₃)₃, and —CH₂Si(CH₃)₃. Specifically included within the definition of “heteroalkyl” are those heteroalkyl groups that are optionally substituted.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule. A cycloalkyl group and a heteroalkyl group can include one or more carbon-carbon double bonds, and/or one or more carbon-carbon triple bonds. Examples of cycloalkyl groups include cyclopropyl, cyclobutyl, cyclopentyl, cyclohexyl, cyclohexenyl, and cycloheptyl groups. Examples of heterocyclic groups include indole, azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, and the like. Specifically included within the definition of “cycloalkyl” are those cycloalkyl groups that are optionally substituted. Specifically included within the definition of “heterocyclyl” are those heterocycle groups that are optionally substituted.

The term “aryl” includes aromatic groups, and is not limited to phenyl, α-naphthyl, β-naphthyl, biphenyl, anthryl, tetrahydronaphthyl, fluorenyl, indanyl, biphenylenyl, and acenaphthenyl. In certain aspects an aryl group is a phenyl group. Specifically included within the definition of “aryl” are those aromatic groups that are optionally substituted. For example, in some aspects of the present invention, the “aryl” groups are optionally substituted with from 1 to 5 substituents selected from the group consisting of hydrogen, hydroxy, aryl, acyl, C₁-C₆ alkyl, C₁-C₆ alkoxy, C₂-C₆ alkenyl, C₂-C₆ alkynyl, substituted alkyl, substituted alkoxy, substituted alkenyl, substituted alkynyl, amino, amino substituted by one or two C₁-C₆ alkyl groups, cyano, halogen, nitro, and trihalomethyl. In some aspects of the present invention, for example, in some aspects wherein the aryl group is phenyl, the aryl groups are optionally substituted with from 1 to 5 substituents selected from the group consisting of hydrogen, hydroxy, aryl, acyl, C₁-C₆ alkoxy, C₂-C₆ alkenyl, C₂-C₆ alkynyl, substituted alkyl, substituted alkoxy, substituted alkenyl, substituted alkynyl, amino, amino substituted by one or two C₁-C₆ alkyl groups, cyano, halogen, nitro, and trihalomethyl.

The term “heteroaryl” refers to an aryl group that contains one to four heteroatoms selected from N, O, and S. Specifically included within the definition of “heteroaryl” are those heteroaryl groups that are optionally substituted. A heteroaryl group can be attached to the remainder of the molecule through a carbon or heteroatom. Non-limiting examples of aryl and heteroaryl groups include phenyl, 4-azaindole, 5-azaindole, 6-azaindole, 7-azaindole, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl. Specifically included within the definition of “alkoxy” are those alkoxy groups that are optionally substituted.

Various groups, including alkyl, heteroalky, cycloalkyl, heterocyclyl, aryl, heteroaryl, and alkoxy, are described herein as substituted or unsubstituted (i.e., optionally substituted). Optionally substituted groups may include one or more substituents independently selected from: —CF₃, —OCF₃, halogen, nitro, cyano, hydroxy, amino, amido, mercapto, formyl, carboxy, oxo, acyl, carbamoyl, silyl, substituted or unsubstituted alkyl, substituted or unsubstituted heteroalkyl, alkoxy, alkenyloxy, alkynloxy, aryloxy, acyloxy, alkylthio, alkylamino, alkenylamino, alkynlamino, arylamino, (alkyl)₂amino, alkylthio, alkenylthio, alkynylthio, arylthio, alkylsulfinyl, sulfonyl, alkylsulfonyl, alkenylsulfonyl, alkynylsulfonyl, arylsulfonyl, acylthio, alkylsilyl, phosphate, phosphonate, phosphinate, cycloalkyl, heterocyclyl, aryl, and heteroaryl. In certain aspects the substituents may be further substituted with one or more substituents.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “acyl” includes straight-chain acyl, branched-chain acyl, cycloacyl, cyclic acyl, heteroatom-unsubstituted acyl, heteroatom-substituted acyl, alkylcarbonyl, alkoxycarbonyl and aminocarbonyl groups. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted acyl” refers to a radical, having a single carbon atom as the point of attachment, the carbon atom being part of a carbonyl group, further having a linear or branched, cyclic or acyclic structure, wherein each additional heteroatom is independently selected from the group consisting of N, O, F, Cl, Br, I, Si, P, and S. The groups, —C(O)CH₂CF₃, —CO₂H, —CO₂, —CO₂CH₃, —CO₂CH₂CH₃, —CO₂CH₂CH₂CH₃, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted alkoxy, and heteroatom-substituted alkoxy. The term “heteroatom-unsubstituted alkoxy” refers to a group, having the structure —OR, in which R is a heteroatom-unsubstituted alkyl. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted alkoxy” refers to a group, having the structure —OR, in which R is a heteroatom-substituted alkyl. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, and heteroatom-substituted alkenyloxy. The term “heteroatom-unsubstituted alkenyloxy” refers to a group, having the structure OR, in which R is a heteroatom-unsubstituted alkenyl. The term “heteroatom-substituted alkenyloxy” refers to a group, having the structure —OR, in which R is a heteroatom-substituted alkenyl.

The term “alkynyloxy” includes straight-chain alkynyloxy, branched-chain alkynyloxy, cycloalkynyloxy, cyclic alkynyloxy, heteroatom-unsubstituted alkynyloxy, and heteroatom-substituted alkynyloxy. The term “heteroatom-unsubstituted alkynyloxy” refers to a group, having the structure —OR, in which R is a heteroatom-unsubstituted alkynyl. The term “heteroatom-substituted alkynyloxy” refers to a group, having the structure —OR, in which R is a heteroatom-substituted alkynyl.

The term “aryloxy” includes heteroatom-unsubstituted aryloxy, heteroatom-substituted aryloxy, heteroaryloxy, and heterocyclic aryloxy groups. The term “heteroatom-unsubstituted aryloxy” refers to a group, having the structure —OAr, in which Ar is a heteroatom-unsubstituted aryl. A non-limiting example of a heteroatom-unsubstituted aryloxy group is —OC₆H₅. The term “heteroatom-substituted aryloxy” refers to a group, having the structure —OAr, in which Ar is a heteroatom-substituted aryl.

The term “acyloxy” includes straight-chain acyloxy, branched-chain acyloxy, cycloacyloxy, cyclic acyloxy, heteroatom-unsubstituted acyloxy, heteroatom-substituted acyloxy, alkylcarbonyloxy, arylcarbonyloxy, alkoxycarbonyloxy, aryloxycarbonyloxy, and carboxylate groups. The term “heteroatom-unsubstituted acyloxy” refers to a group, having the structure —OAc, in which Ac is a heteroatom-unsubstituted acyl. For example, —OC(O)CH₃ is a non-limiting example of a heteroatom-unsubstituted acyloxy group. The term “heteroatom-substituted acyloxy” refers to a group, having the structure —OAc, in which Ac is a heteroatom-substituted acyl. For example, —OC(O)OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, and heteroatom-substituted alkylamino. The term “heteroatom-unsubstituted alkylamino” refers to a radical, having a single nitrogen atom as the point of attachment, further having one or two saturated carbon atoms attached to the nitrogen atom, further having a linear or branched, cyclic or acyclic structure, 4 or more hydrogen atoms, a total of 1 nitrogen atom, and no additional heteroatoms. The term “heteroatom-unsubstituted alkylamino” includes groups, having the structure —NHR, in which R is a heteroatom-unsubstituted alkyl. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. The term “heteroatom-substituted alkylamino” includes groups, having the structure —NHR, in which R is a heteroatom-substituted alkyl.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, dialkenylamino, and alkyl(alkenyl)amino groups. The term “heteroatom-unsubstituted alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, 4 or more hydrogen atoms, a total of one nitrogen atom, and no additional heteroatoms. The term “heteroatom-unsubstituted alkenylamino” includes groups, having the structure —NHR, in which R is a heteroatom-unsubstituted alkenyl. The term “heteroatom-substituted alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. The term “heteroatom-substituted alkenylamino” includes groups, having the structure —NHR, in which R is a heteroatom-substituted alkenyl.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, dialkynylamino, alkyl(alkynyl)amino, and alkenyl(alkynyl)amino groups. The term “heteroatom-unsubstituted alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, at least one hydrogen atoms, a total of one nitrogen atom, and no additional heteroatoms. The term “heteroatom-unsubstituted alkynylamino” includes groups, having the structure —NHR, in which R is a heteroatom-unsubstituted alkynyl. The term “heteroatom-substituted alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. The term “heteroatom-substituted alkynylamino” includes groups, having the structure NHR, in which R is a heteroatom-substituted alkynyl.

The term “arylamino” includes heteroatom-unsubstituted arylamino, heteroatom-substituted arylamino, heteroarylamino, heterocyclic arylamino, and alkyl(aryl)amino groups. The term “heteroatom-unsubstituted arylamino” refers to a radical, having a single nitrogen atom as the point of attachment, further having at least one aromatic ring structure attached to the nitrogen atom, wherein the aromatic ring structure contains only carbon atoms, 6 or more hydrogen atoms, a total of one nitrogen atom, and no additional heteroatoms. The term “heteroatom-unsubstituted arylamino” includes groups, having the structure —NHR, in which R is a heteroatom-unsubstituted aryl. The term “heteroatom-substituted arylamino” refers to a radical, having a single nitrogen atom as the point of attachment, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. The term “heteroatom-substituted arylamino” includes groups, having the structure —NHR, in which R is a heteroatom-substituted aryl.

The term “amido” includes straight-chain amido, branched-chain amido, cycloamido, cyclic amido, heteroatom-unsubstituted amido, heteroatom-substituted amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted amido” refers to a radical, having a single nitrogen atom as the point of attachment, further having a carbonyl group attached via its carbon atom to the nitrogen atom, further having a linear or branched, cyclic or acyclic structure, 1 or more hydrogen atoms, a total of one oxygen atom, a total of one nitrogen atom, and no additional heteroatoms. The term “heteroatom-unsubstituted amido” includes groups, having the structure —NHR, in which R is a heteroatom-unsubstituted acyl. The group, —NHC(O)CH₃, is a non-limiting example of a heteroatom-unsubstituted amido group. The term “heteroatom-substituted amido” refers to a radical, having a single nitrogen atom as the point of attachment, further having a carbonyl group attached via its carbon atom to the nitrogen atom, further having a linear or branched, cyclic or acyclic structure, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. The term “heteroatom-substituted amido” includes groups, having the structure —NHR, in which R is a heteroatom-unsubstituted acyl. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, and heteroatom-substituted alkylthio. The term “heteroatom-unsubstituted alkylthio” refers to a group, having the structure —SR, in which R is a heteroatom-unsubstituted alkyl. The group, —SCH₃, is an example of a heteroatom-unsubstituted alkylthio group. The term “heteroatom-substituted alkylthio” refers to a group, having the structure —SR, in which R is a heteroatom-substituted alkyl.

The term “alkenylthio” includes straight-chain alkenylthio, branched-chain alkenylthio, cycloalkenylthio, cyclic alkenylthio, heteroatom-unsubstituted alkenylthio, and heteroatom-substituted alkenylthio. The term “heteroatom-unsubstituted alkenylthio” refers to a group, having the structure —SR, in which R is a heteroatom-unsubstituted alkenyl. The term “heteroatom-substituted alkenylthio” refers to a group, having the structure —SR, in which R is a heteroatom-substituted alkenyl.

The term “alkynylthio” includes straight-chain alkynylthio, branched-chain alkynylthio, cycloalkynylthio, cyclic alkynylthio, heteroatom-unsubstituted alkynylthio, and heteroatom-substituted alkynylthio. The term “heteroatom-unsubstituted alkynylthio” refers to a group, having the structure —SR, in which R is a heteroatom-unsubstituted alkynyl. The term “heteroatom-substituted alkynylthio” refers to a group, having the structure —SR, in which R is a heteroatom-substituted alkynyl.

The term “arylthio” includes heteroatom-unsubstituted arylthio, heteroatom-substituted arylthio, heteroarylthio, and heterocyclic arylthio groups. The term “heteroatom-unsubstituted arylthio” refers to a group, having the structure —SAr, in which Ar is a heteroatom-unsubstituted aryl. The group, —SC₆H₅, is an example of a heteroatom-unsubstituted arylthio group. The term “heteroatom-substituted arylthio” refers to a group, having the structure —SAr, in which Ar is a heteroatom-substituted aryl.

The term “acylthio” includes straight-chain acylthio, branched-chain acylthio, cycloacylthio, cyclic acylthio, heteroatom-unsubstituted acylthio, heteroatom-substituted acylthio, alkylcarbonyloxy, arylcarbonyloxy, alkoxycarbonyloxy, aryloxycarbonyloxy, and carboxylate groups. The term “heteroatom-unsubstituted acylthio” refers to a group, having the structure —SAc, in which Ac is a heteroatom-unsubstituted acyl. The group, —SCOCH₃, is an example of a heteroatom-unsubstituted acylthio group. The term “heteroatom-substituted acylthio” refers to a group, having the structure —SAc, in which Ac is a heteroatom-substituted acyl.

The term “alkylsilyl” includes straight-chain alkylsilyl, branched-chain alkylsilyl, cycloalkylsilyl, cyclic alkylsilyl, heteroatom-unsubstituted alkylsilyl, and heteroatom-substituted alkylsilyl. The term “heteroatom-unsubstituted alkylsilyl” refers to a radical, having a single silicon atom as the point of attachment, further having one, two, or three saturated carbon atoms attached to the silicon atom, further having a linear or branched, cyclic or acyclic structure, 5 or more hydrogen atoms, a total of 1 silicon atom, and no additional heteroatoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S.

The term “phosphonate” includes straight-chain phosphonate, branched-chain phosphonate, cyclophosphonate, cyclic phosphonate, heteroatom-unsubstituted phosphonate, and heteroatom-substituted phosphonate. The term “heteroatom-unsubstituted phosphonate” refers to a radical, having a single phosphorous atom as the point of attachment, further having a linear or branched, cyclic or acyclic structure, 2 or more hydrogen atoms, a total of three oxygen atom, and no additional heteroatoms. The three oxygen atoms are directly attached to the phosphorous atom, with one of these oxygen atoms doubly bonded to the phosphorous atom. The groups, —P(O)(OH)₂, —P(O)(OH)OCH₃, —P(O)(OH)OCH₂CH₃, —P(O)(OCH₃)₂, and —P(O)(OH)(OC₆H₅) are non-limiting examples of heteroatom-unsubstituted phosphonate groups. The term “heteroatom-substituted phosphonate” refers to a radical, having a single phosphorous atom as the point of attachment, further having a linear or branched, cyclic or acyclic structure, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S.

The term “phosphinate” includes straight-chain phosphinate, branched-chain phosphinate, cyclophosphinate, cyclic phosphinate, heteroatom-unsubstituted phosphinate, and heteroatom-substituted phosphinate. The term “heteroatom-unsubstituted phosphinate” refers to a radical, having a single phosphorous atom as the point of attachment, further having a linear or branched, cyclic or acyclic structure, 2 or more hydrogen atoms, a total of two oxygen atom, and no additional heteroatoms. The two oxygen atoms are directly attached to the phosphorous atom, with one of these oxygen atoms doubly bonded to the phosphorous atom. The groups, —P(O)(OH)H, —P(O)(OH)CH₃, —P(O)(OH)CH₂CH₃, —P(O)(OCH₃)CH₃, and —P(O)(OC₆H₅)H are non-limiting examples of heteroatom-unsubstituted phosphinate groups. The term “heteroatom-substituted phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S.

Any apparently unfulfilled valency is to be understood to be properly filled by hydrogen atom(s). For example, a compound with a substituent of —O or —N is to be understood to be —OH or —NH₂, respectively.

The term “geminal” refers to a carbon atom to which two atoms or functional groups are attached. A geminal diol, for example, is a compound in which two hydroxyl groups are attached to the same carbon atom.

Any genus, subgenus, or specific compound discussed herein is specifically contemplated as being excluded from any aspect described herein.

Compounds described herein may be prepared synthetically using conventional organic chemistry methods known to those of skill in the art and/or are commercially available (e.g., ChemBridge Co., San Diego, Calif.; MCule, Palo Alto, CA; etc.).

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Derivatives of compounds of the present invention are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds of the present invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some aspects, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers. Compounds of the present invention may occur as a hydrate, a compound containing an equivalent of water in the form of an H₂O molecule, or polyhydrate, a compound containing more than one equivalent of water in the form of H₂O molecules.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

As noted above, compounds of the present invention may exist in prodrug form. As used herein, “prodrug” is intended to include any covalently bonded carriers which release the active parent drug or compounds that are metabolized in vivo to an active drug or other compounds employed in the methods of the invention in vivo when such prodrug is administered to a subject. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### BRIEF DESCRIPTION OF THE FIGURES

FIGS. 1A-1B display TCGA data showing elevated expression of NSUN1 and NSUN2 in patients with many cancer subtypes (FIG. 1A), and exemplary correlation of NSUN1 and NSUN2 expression with significantly shorter survival in patients with renal cancer (FIG. 1B).

FIG. 2 displays a schematic exemplifying subcellular locations of NSUN1 and NSUN2 in a cell.

FIGS. 3A-3C display a schematic exemplifying RCMT-mediated (e.g., NSUN1 and/or NSUN2 mediated) active chromatin structures (ACS) (FIG. 3A), and at ACS with nascent RNAs in drug resistant cells (e.g., MDS/AML cells) (FIG. 3B). FIG. 3C provide insights into NSUN1-mediated drug-resistant active transcription complex by co-immunoprecipitation.

FIGS. 4A-4B display exemplary results of NSUN1/2/ACS imaging/quantification methods. FIG. 4A shows NSUN1/2-ACS-targeting PL-RCA/CM imaging results. FIG. 4B shows the results of EC-FCM for quantifying nascent RNA synthesis.

FIGS. 5A-5G display a pipeline of computational design and functional screening of small-molecule compound libraries as well as exemplary characterization and validation of identified positive hit compounds and their targets. FIGS. 5A-5D display an exemplary experimental procedure, beginning with characterization of NSUN1 and/or NSUN2 putative binding modules for interaction with ACS components (FIGS. 5A-5B), a graphic displaying the design of a small molecule library and testing of the putative small molecule NSUN1/2-ACS inhibitors in cell lines (FIG. 5C), characterization of the NSUN1/2-ACS inhibitors interactions with the ACS machinery (FIG. 5D). FIG. 5E graphs the inhibition of the growth of venetoclax-resistant SC cells by exemplary positive hits NSUN1-17 and NSUN1-11. FIG. 5F shows computationally predicted docking of NSUN1-17 and NSUN1-11 in A-460 binding module of NSUN1. FIG. 5G shows measurements of the effects of NSUN1-17 on the interactions between NSUN1 and other key components in the drug (venetoclax)-resistant ACS (e.g., that NSUN1-17 inhibited the interaction of NSUN1 with each of eRNAPII, MYC, and BRD4, but not the interaction between NSUN1 and RNA m5C).

FIGS. 6A-6D display an exemplary screening of NSUN1/2-targeting drug libraries and quantification of NSUN1/2-mediated ACS in cancer cells (e.g., Leukemia). FIGS. 6A-6B are schematic presentations of NSUN1/2-targeting fluorescence quenching release (FQR) plate assay and PL-RCA-FCM respectively. FIGS. 6C-6D display PL-RCA-FCM measurement of NSUN2-mediated interactions with m5C, eRNAPII, i.e., eRNAPII (RNAPII-CTD-S2P) and BRD4 in the SC and K1, respectively. Black: NSUN2 antibody only (neg ctrl); Red, green and blue: Addition of antibody against m5C, eRNAPII, i.e., RNAPII-CTD-S2P, and BRD4, respectively. Following in silico and in vitro work, lead compounds can be further validated in vivo (e.g., in mice models).

FIGS. 7A-7C display schematics outlining NSUN1/2-ACS-targeting fluorescence quenching release assay (FQRA) results. FIG. 7A depicts a fluorescence quenching complex. FIG. 7B depicts fluorescence quenched in the absence of an inhibitory drug. FIG. 7C depicts the release of quenched fluorescence by presence of an NSUN1/2-ACS inhibitory drug.

FIGS. 8A-8B display a schematic of an 5-ethynyl uridine click chemistry (EC)-coupled flow cytometry (EC-FCM) assay (FIG. 8A), while FIG. 8B depicts the results of an EC-FCM assay showing distinct drug-specific signatures/patterns of nascent RNA synthesis inhibition in the venetoclax sensitive leukemia cell line (K1). The MTT assay measures cell viability and proliferation as well as cytotoxicity of compounds by assessing cellular metabolic activity. The associated measurement can be described as cell viability, cell killing, and/or growth inhibition.

FIG. 9 displays a schematic representation of an assay (as described in FIGS. 8A-8B) flowthrough.

FIGS. 10A-10M display binding pockets for NSUN1 and NSUN2 interactions with ACS. Specifically for NSUN1, modules: A-219 (FIG. 10G; module 1), A-316 (FIG. 10A; module 2), A-371 (FIG. 10H; module 3) and A-460 (FIG. 10B; module 4); and specifically for NSUN2, modules A-89 (FIG. 10J; module 1), A-162 (FIG. 10K; module 2), A-202 (FIG. 10L; module 3), A-271 (FIG. 10E; module 4), A-279 (FIG. 10F; module 5), A-301 (FIG. 10I; module 6), A-580 (FIG. 10C; module 7), A-691 (FIG. 10D; module 8), and A-700 (FIG. 10M; module 9).

FIGS. 11A-11D display scatter plot representations of screening results for AI-designed NSUN1-targeting compound libraries in a venetcoclax-resistant human myeloid leukemia cell line (SC), lung cancer cell line (H₈₂), and venetoclax resistant human lung cancer cell line (K562), and a schematic representation of categorization of the results. A human myeloid leukemia cell Line (SC), a lung cancer cell line (H₈₂ or K562) were both incubated at drug concentration 10 μM/μL overnight. MTT assays were performed after 24 hours, and changes were calculated as the percentage of mean absorbance (n=3) versus mean absorbance of drug-free control (n=3). Negative outliers indicated in red ovals. FIG. 11A depicts the results of an NSUN1-targeting drug library, 74 drugs, comparing growth/inhibition in H-82 (Y axis) and SC (X axis) cell lines. FIG. 11B depicts the results of an NSUN2-targeting drug library, 102 drugs, comparing growth/inhibition in H-82 (Y axis) and SC (X axis) cell lines. FIG. 11C depicts the results of an NSUN1-targeting drug library, 74 drugs, comparing growth/inhibition in K562 (Y axis) and SC (X axis) cell lines. FIG. 11D is a schematic representation showing categorization of the results from screens performed as described in FIG. 11, and validation experiments as described in FIG. 12.

FIGS. 12A-12J display bar graphs displaying the results of validation experiments for small molecule inhibitors of NSUN1/2-ACS interactions as identified herein. Drug validation of select SC, H82, and/or K562 inhibiting compounds in mouse acute myeloid leukemia cell Line (C1498), SC cells, H82 cells, and/or K562 cells. NSUN1-17 and/or venetoclax acted as controls. Inhibition of cell vitality was identified as dose-dependent for certain small molecules, not merely cytotoxic. In brief, stock with concentration 0.4×10\hat{ }6 test (e.g., SC, H82, K562, or C1498, as labelled) cells/mL, and 10 μM/μL drug was serially diluted into drug-free cell stock to reach concentrations of 1 μM/μL and 0.1 μM/mL and split into 100 μL volumes in a 96-well plate. MTT assays performed after 24 hour incubation. Percentages calculated as a difference in mean absorbance of each drug triplicate and mean absorbance in drug-free cell triplicate. Red bars represent standard errors. The concentration of drug represented by each column is from left to right as written in the figure keys. FIGS. 12A-12E provide results from independent repeats for different groupings of small molecules tested against C1498 cells. FIGS. 12F-12G provide results from independent repeats for different groupings of small molecules tested against H82 cells. FIGS. 12H-12J provide results from independent repeats for different groupings of small molecules tested against SC cells. FIG. 12J provide results from an assay against K562 cells.

FIGS. 13A-13L display 2D chemical structures, InChIKey data, SMILES data, and formula data for certain NSUN1/2-ACS inhibitors (selected from NSUN1 binding library) identified as positive hits for inhibiting tumor cell growth in a lineage independent manner.

FIGS. 14A-14N display 2D chemical structures, InChIKey data, SMILES data, and formula data for certain NSUN1/2-ACS inhibitors (selected from NSUN1 binding library) identified as positive hits for inhibiting tumor cell growth in a lineage dependent manner (FIGS. 14A-14J were leukemia-specific compounds; FIGS. 14K-14N were lung cancer-specific compounds).

FIGS. 15A-15M display 2D chemical structures, InChIKey data, SMILES data, and formula data for certain NSUN1/2-ACS inhibitors (selected from NSUN2 binding library) identified as positive hits for inhibiting tumor cell growth in a lineage independent manner.

FIGS. 16A-16N display 2D chemical structures, InChIKey data, SMILES data, and formula data for certain NSUN1/2-ACS inhibitors (selected from NSUN2 binding library) as positive hits for inhibiting tumor cell growth in a lineage dependent manner (FIGS. 16A-16H were leukemia-specific compounds; FIGS. 16I-16N were lung cancer-specific compounds).

FIGS. 17A-17Y display 2D chemical structures, InChIKey data, SMILES data, and formula data for certain NSUN1/2-ACS binding molecules (selected from NSUN1 and NSUN2 binding library) as hits for promoting tumor cell growth in a lineage independent or dependent manner.

FIGS. 18A-18E display 2D chemical structures for certain NSUN1/2-ACS inhibitors identified as positive hits for inhibiting tumor cell growth in a lineage dependent or independent manner.

FIGS. 19A-19B provide in vivo validation results for NSUN1/2-targeting compound NSUN1-17 in the lung tissue of syngeneic mouse model comprising mouse leukemia cell line C₁₄₉₈.

FIGS. 20A-20F provide a schematic illustration of the principles (FIGS. 20A and 20B) of NSUN1/2-targeting Fluorescence Quenching Release (NSUN1/2-FQR) high-throughput assay for screening NSUN1/2 drug libraries, two methods (FIGS. 20C and 20D) for conducting the same, and the design (FIGS. 20E and 20F) of the associated fluorescence quenching linker complexes, SEQ ID NOs: 3-6, with 5′ modifications Bi, IBQ, or BioTEG respectively.

FIG. 21 provide select results for Hit-to-Lead and structure-activity relationship analysis of NSUN1/2 compounds NSUN1-17 and NSUN2-11. The data shows cell viability of venetoclax-resistant monocytic leukemia cells exposed to increasing concentrations of NSUN1-17, NSUN2-11, or Venetoclax.

FIG. 22 provides PL-RCA-FCM results and an associated schematic showing that NSUN2-11 can disrupt both the NSUN1- and NSUN2-mediated interactions with c-MYC and RNA polymerase II (RNAPII), while NSUN1-17 can only disrupt the NSUN1-mediated interaction, but not the NSUN-2 mediated interaction. Black=negative control, blue=positive control, no drug=red.

FIG. 23 provides PL-RCA-FCM results and an associated schematic showing that NSUN2-11 can disrupt the interactions between NSUN2 and mitochondrial RNA polymerase (POLRMT) as well as mitochondrial translation initiation factor 3 (MTIF3). RNA:m5C/NSUN1/2-mediated transcription and translation complexes (RNATTC) mediate multidrug resistance through regulating multi-bioprocesses and multi-organelles' functions in cancer cells.

### DETAILED DESCRIPTION

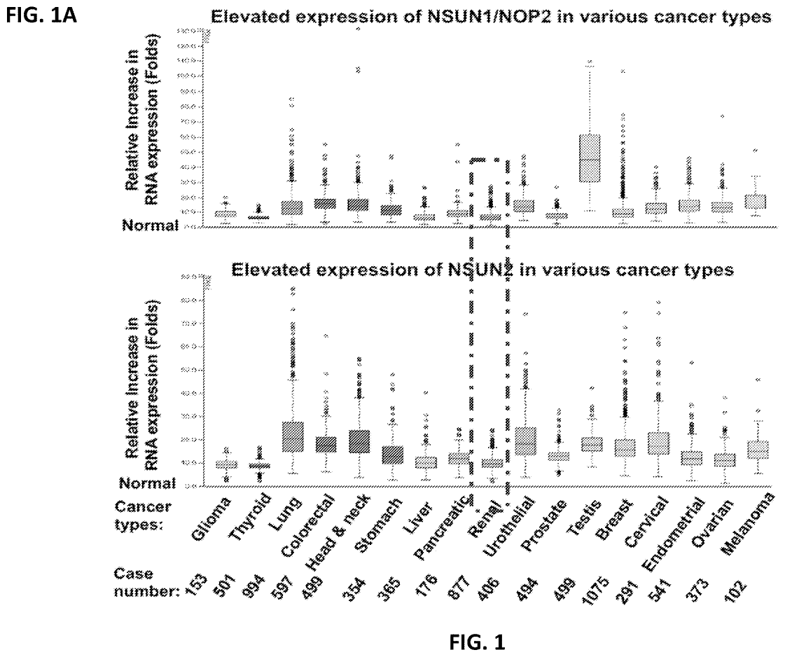

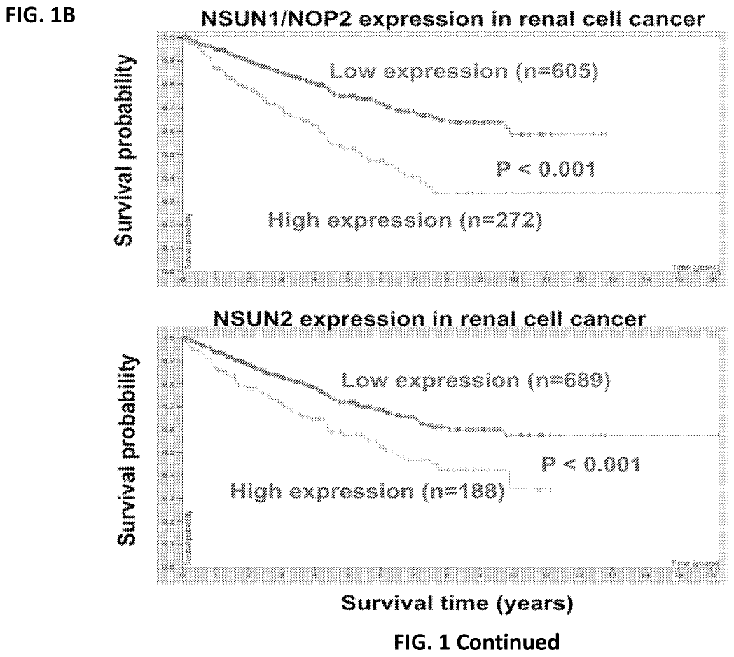

The clinical significance of RNA cytosine methyltransferases NSUN1 and NSUN2 in oncology. TCGA data demonstrated that elevated expression levels of NSUN1 (aka NOP2, NOL1) and NSUN2 are closely correlated with a significantly shorter survival in patients with nearly all cancer subtypes (FIG. 1). NSUN1 and NSUN2 belong to RNA cytosine methyltransferase (RCMT) family that comprises seven NOL1/NOP2/SUN domain (NSUN) proteins and the DNA methyltransferase homologue 2 protein (DNMT2) and catalyze the transfer of methyl groups to the C₅ position of cytosine residues in a wide variety of RNA species, i.e., RNA:m5C), including cytoplasmic and mitochondrial ribosomal RNAs (rRNAs), transfer RNAs (tRNAs), messenger RNAs (mRNAs), enhancer RNAs (eRNAs) and other non-coding RNAs.

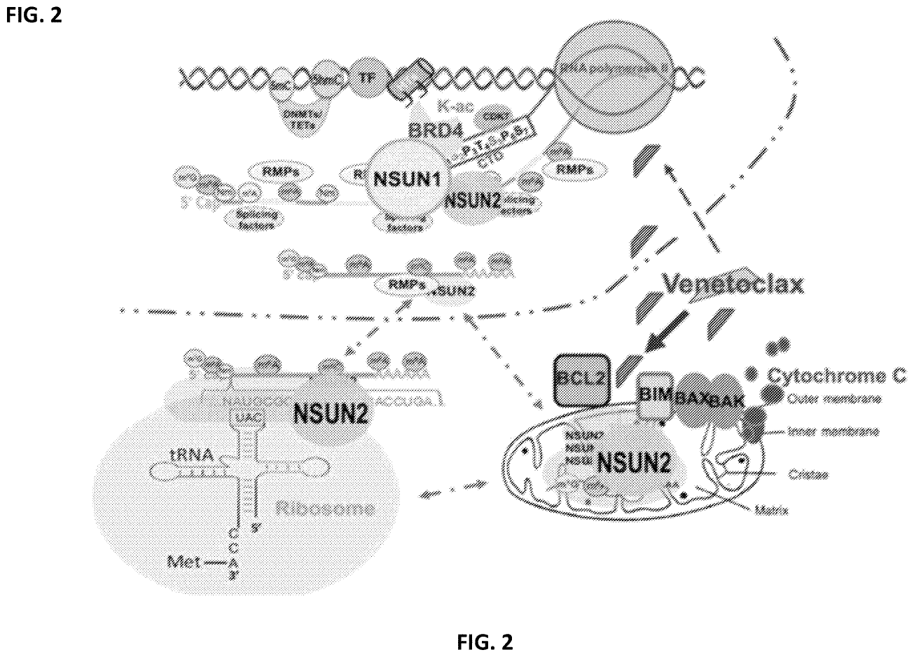

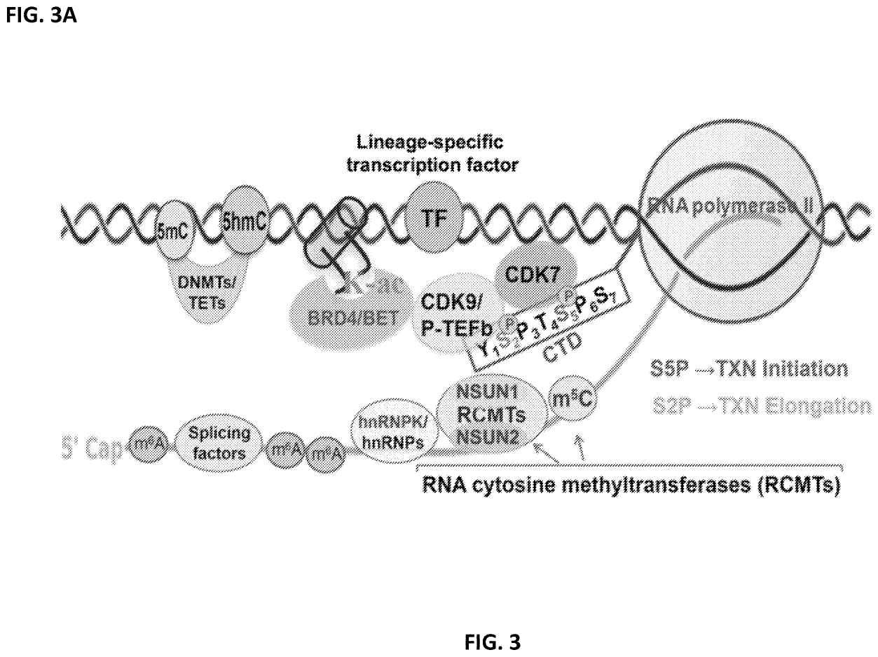

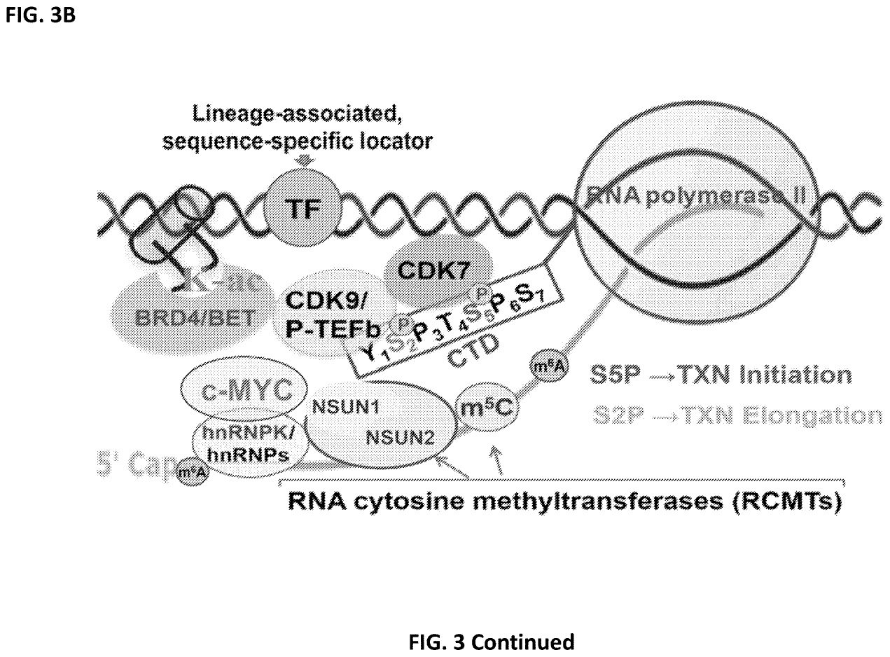

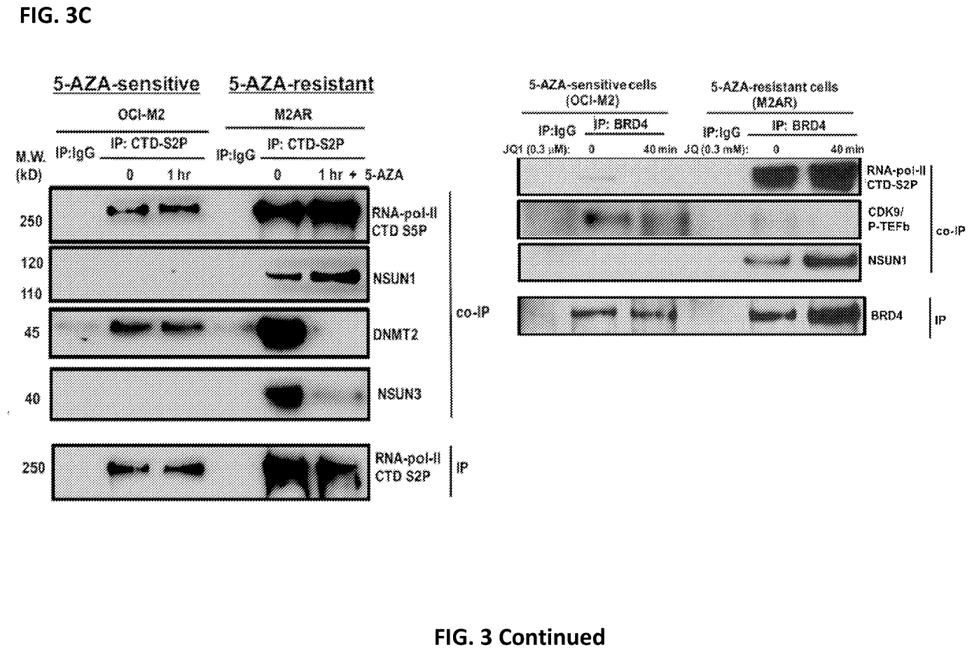

NSUN1 and NSUN2 are responsible for catalyzing the majority of RNA:m5C reactions and regulate chromatin structure, gene expression and organelle functions, as well as cancer progression and drug resistance. NSUN1 and NSUN2 have different subcellular locations; NSUN1 is located in the nucleus and nucleolus, while NSUN2 is distributed in both the nucleus and the cytoplasmic organelles, including ribosomes and mitochondria. Both NSUN1 and NSUN2 can regulate chromatin structure, transcription and translation as well as metabolism and cancer progression and drug resistance (FIGS. 1 and 2). The inventors previously published studies (see e.g., Jason Cheng et al., “RNA cytosine methylation and methyltransferases mediate chromatin organization and 5-azacytidine response and resistance in leukaemia” Nature Communications. 2018; which is incorporated herein by reference in its entirety for any purpose described herein) and unpublished data provided herein demonstrated that NSUN1 and NSUN2 mediate the formation of distinct active chromatin structures (ACS) at nascent RNAs, and such ACS can determine drug (e.g., azacitidine and venetoclax) resistance in leukemia cells. However, the mechanisms underlying NSUN1/2-mediated oncology and drug resistance were largely unknown, and before now (to the inventors knowledge), no NSUN1/2-targeting cancer diagnostics and therapeutics have been developed.

Provided herein is support for the discovery of RCMTs-mediated drug-resistant vs. drug-sensitive active ACS in leukemia cells. Both NSUN1 and NSUN2 regulate multiple essential bioprocesses, but their clinical implications are largely unexplored. The inventors previously demonstrated that RCMTs (e.g., NSUN1 and NSUN2) directly interacted with bromodomain-containing protein 4 (BRD4) and elongating RNA polymerase II (eRNAPII, CTD-S2P) to form distinct active chromatin structures (ACS) at nascent RNAs, and such ACS determined drug (azacitidine) resistance in leukemia cells. This was the first study to demonstrate the presence of distinct drug-resistant ACS in leukemia cells.

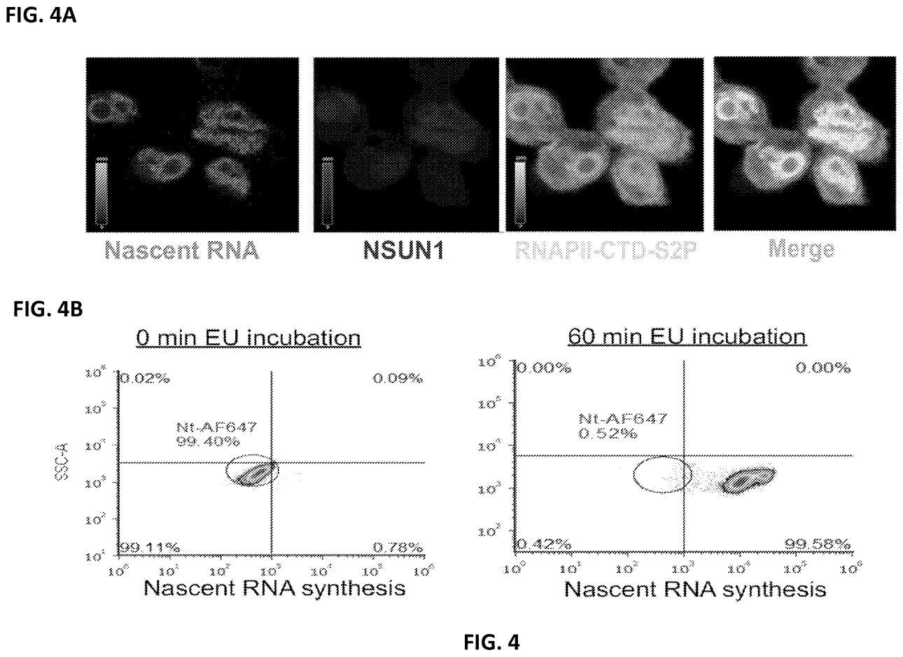

By combining 5-ethynyl uridine click chemistry (EC), proximity ligation rolling cycle amplification (PL-RCA), fluorescent confocal microscopy (CM), and flow cytometry (FCM), the inventors have developed new technologies, specifically PL-RCA-CM (exemplary images provided in FIG. 4A) and EC-/PL-RCA-FCM (exemplary results provided in FIG. 4B) to visualize and quantify drug-induced changes in ACS and nascent RNA synthesis in living cells. The inventors have developed various drug (e.g., azacitidine and venetoclax) cell lines that were shown to have NSUN1 and/or NSUN2 enforced expression cell lines, and Nsun2 heterozygous and homozygous knockout (KO) mice. These cell lines and animal models provide powerful tools to study NSUN1/2 functions and drug effects on NSUN1/2-mediated ACS as well as functional screening and target validation of the NSUN1/2-targeting compound libraries. With these tools in hand, the inventors sought to design novel compounds and/or identify new uses for known compounds with the goal of targeting NSUN1/2-ACS for inhibition.

Computational Design and Functional Screening of Small-Molecule Compound Libraries that target NSUN1 and NSUN2 proteins in leukemia and cancer cells.

In some aspects described herein, initial identification of putative small-molecule NSUN1 and/or NSUN2 inhibitors was provided through a supercomputing AI approach. Recent development of machine learning (ML) and artificial intelligence (AI) enables prediction of protein structures with high accuracy and can greatly accelerate drug discovery. In some aspects, employment of supercomputing AI approaches can prove critically important for developing NSUN1/2-targeting small-molecule inhibitors as the crystal structures of NSUN1 and NSUN2 have not yet been solved.

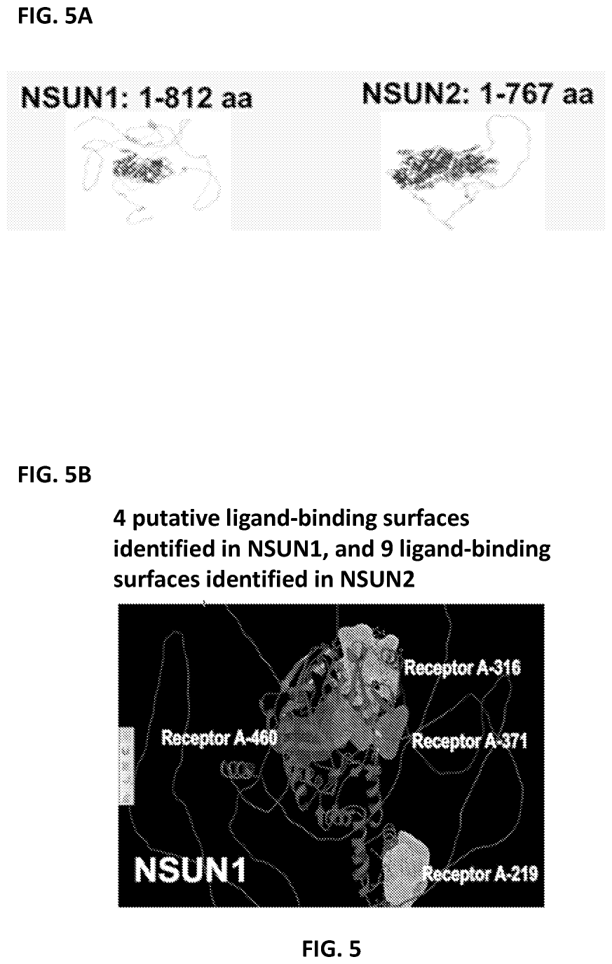

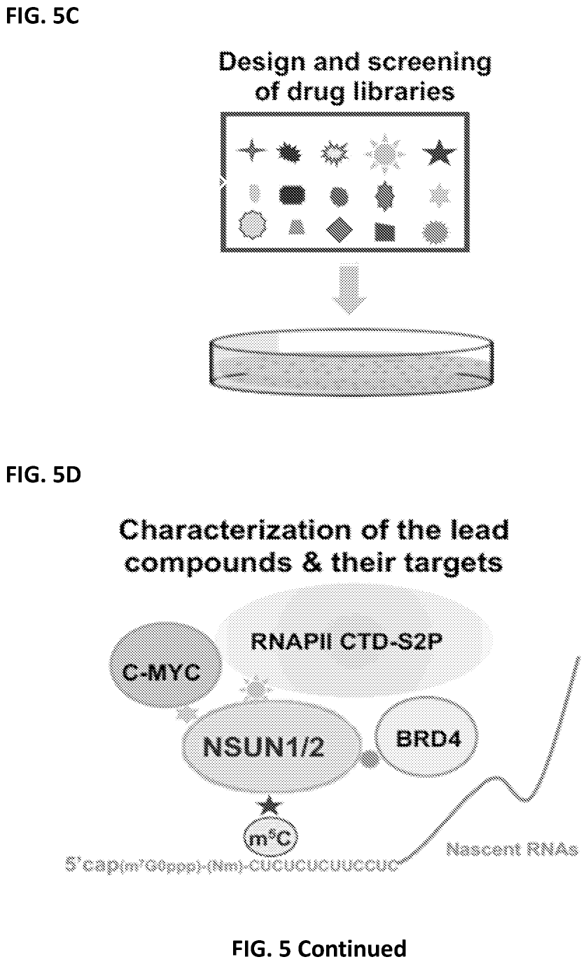

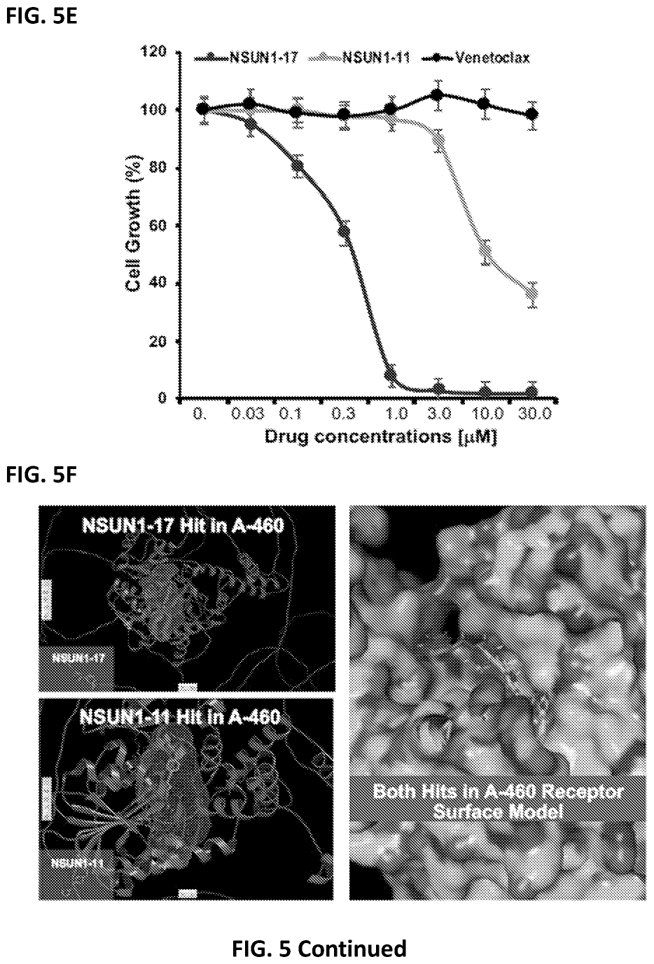

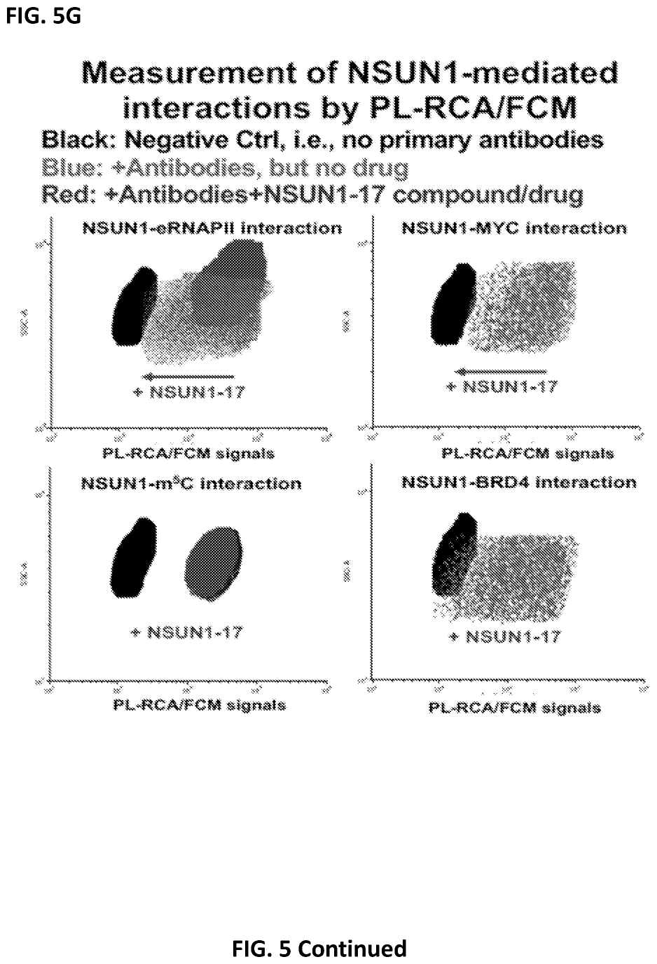

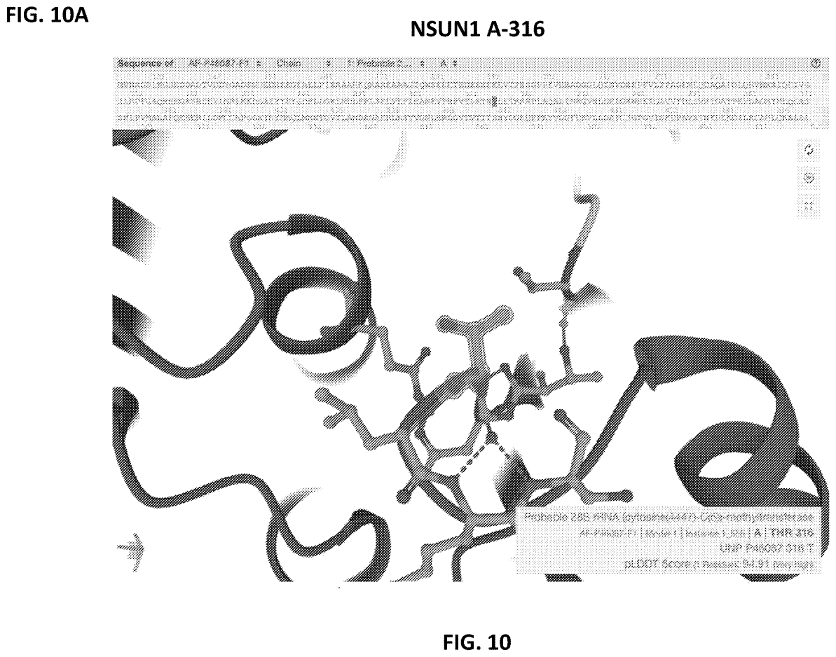

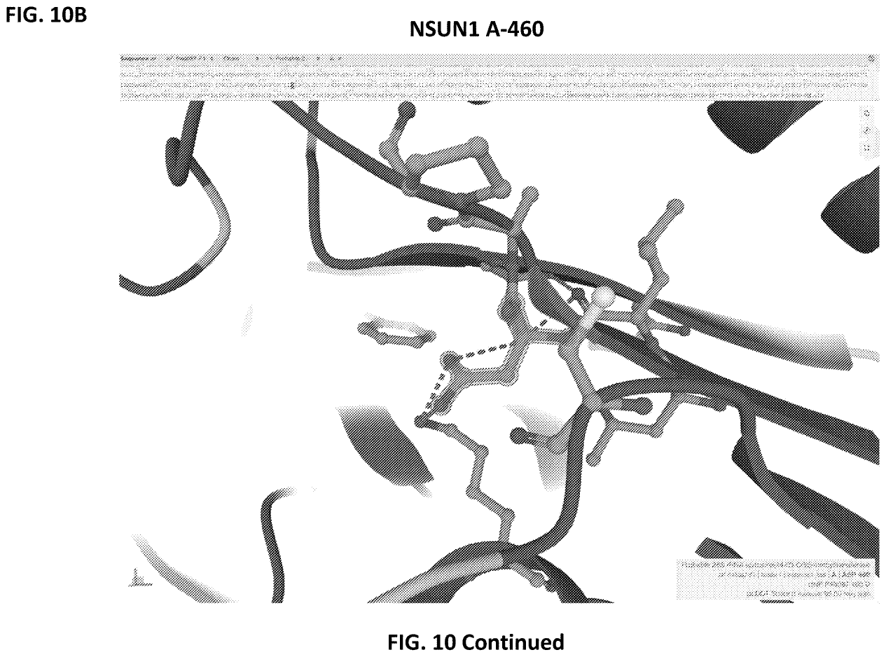

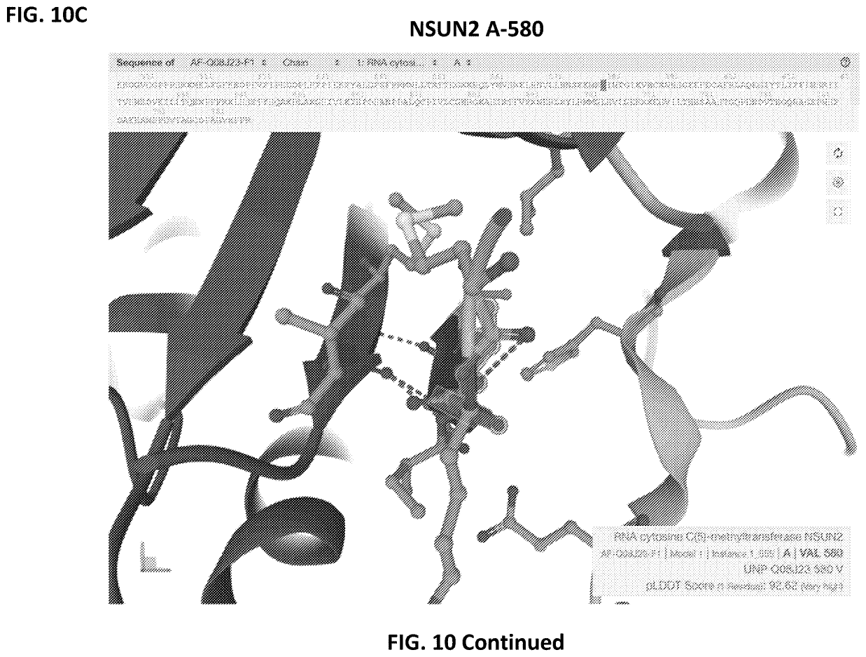

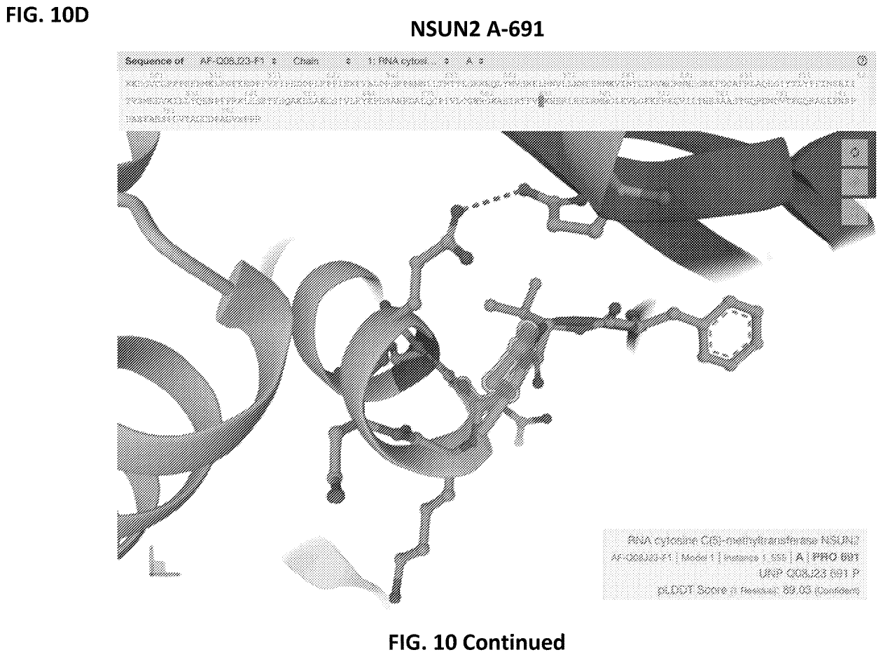

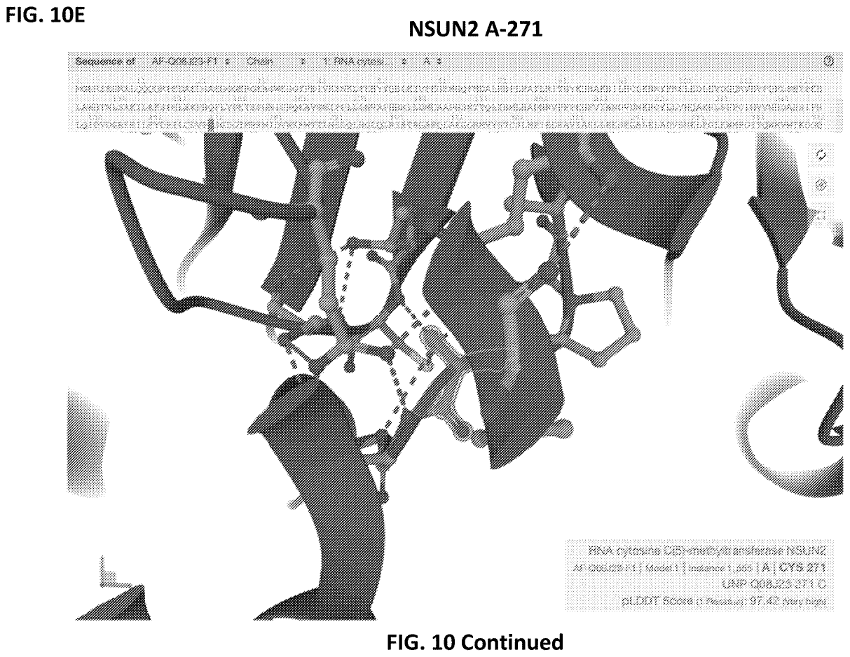

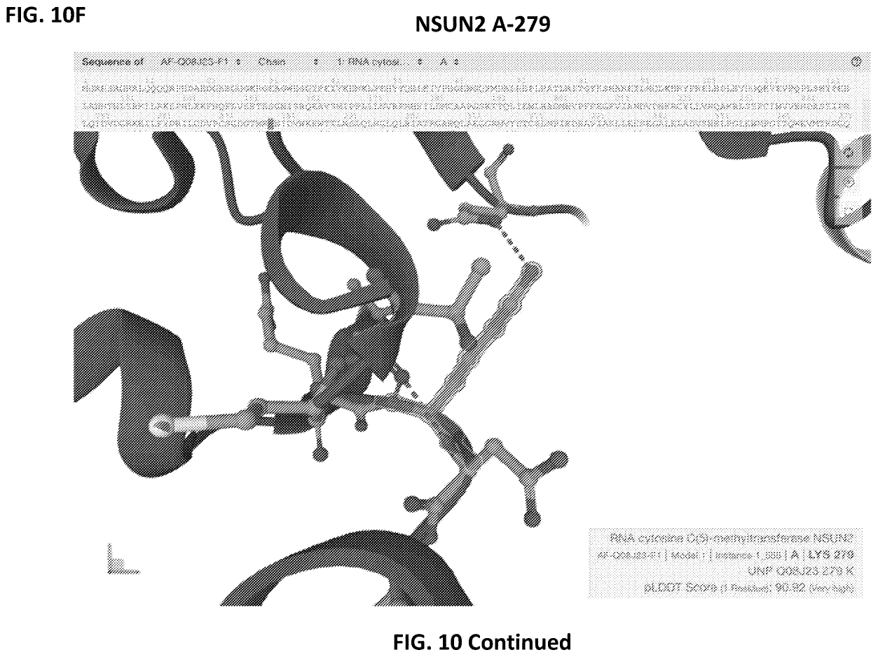

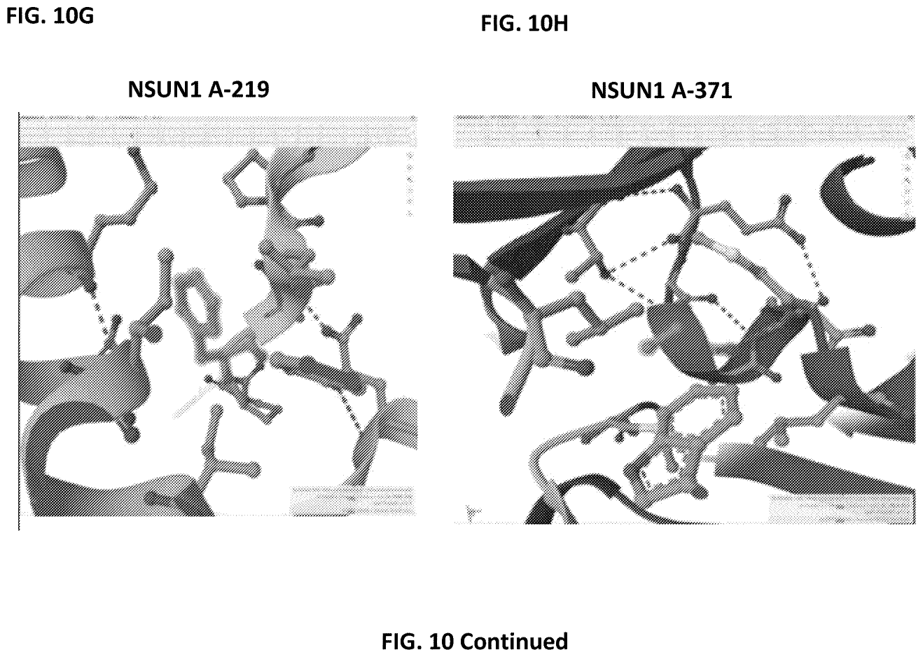

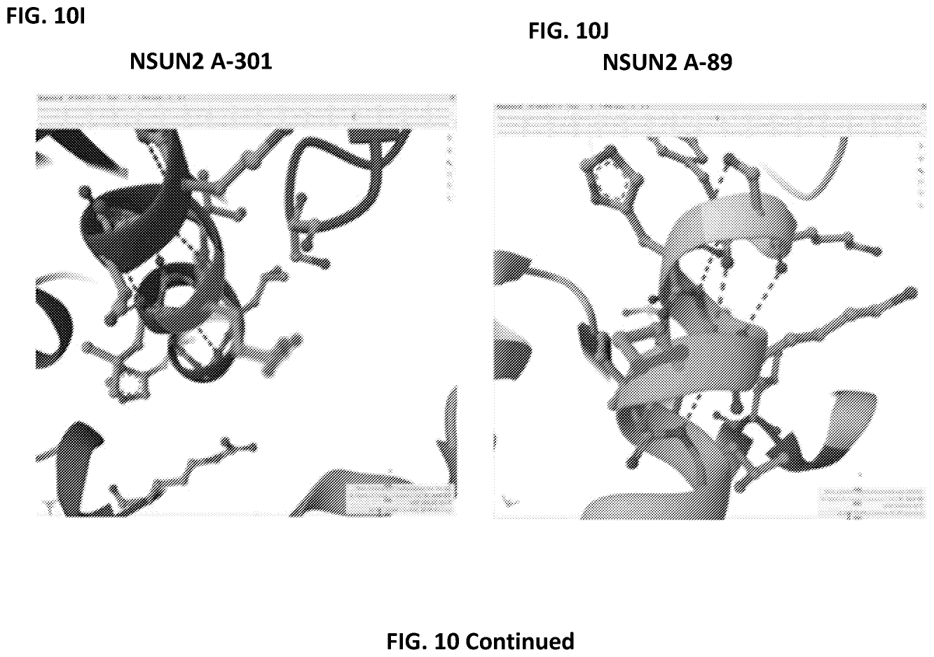

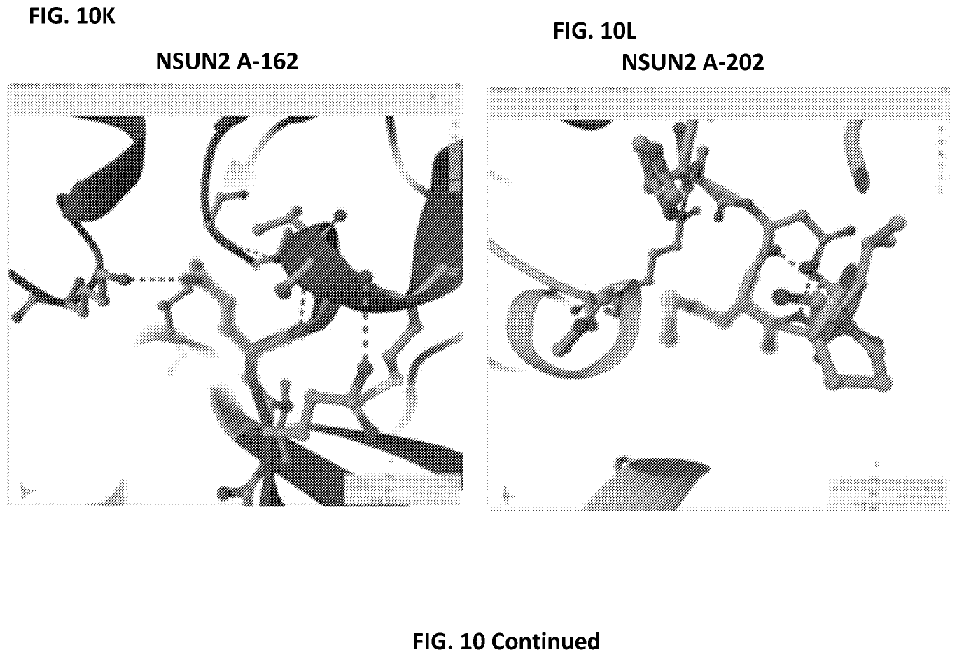

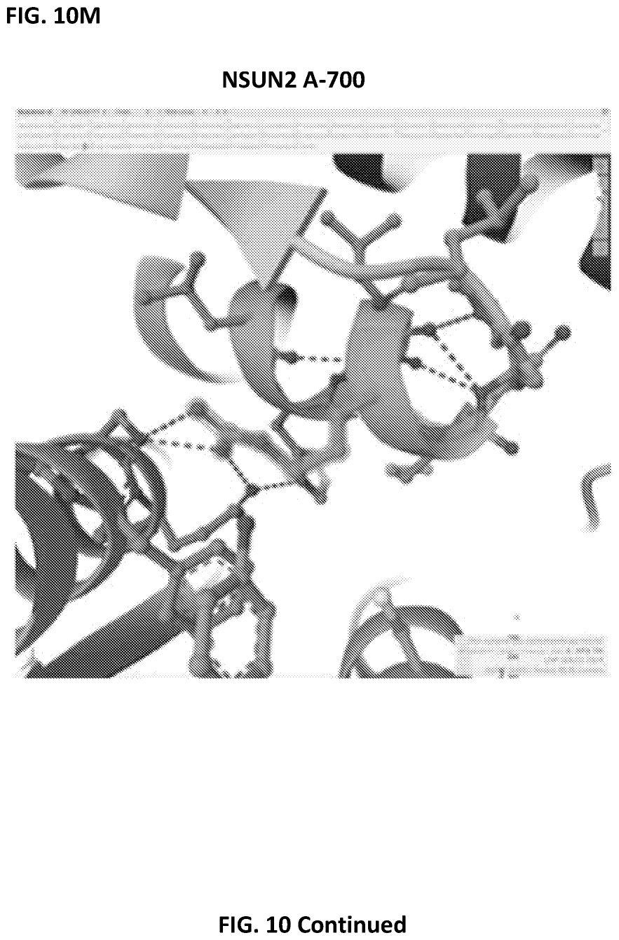

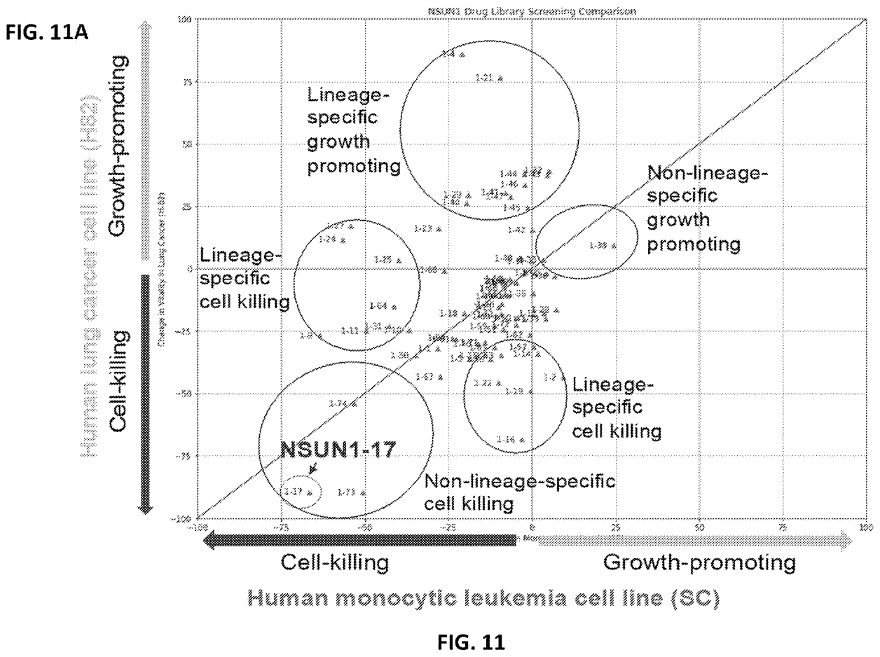

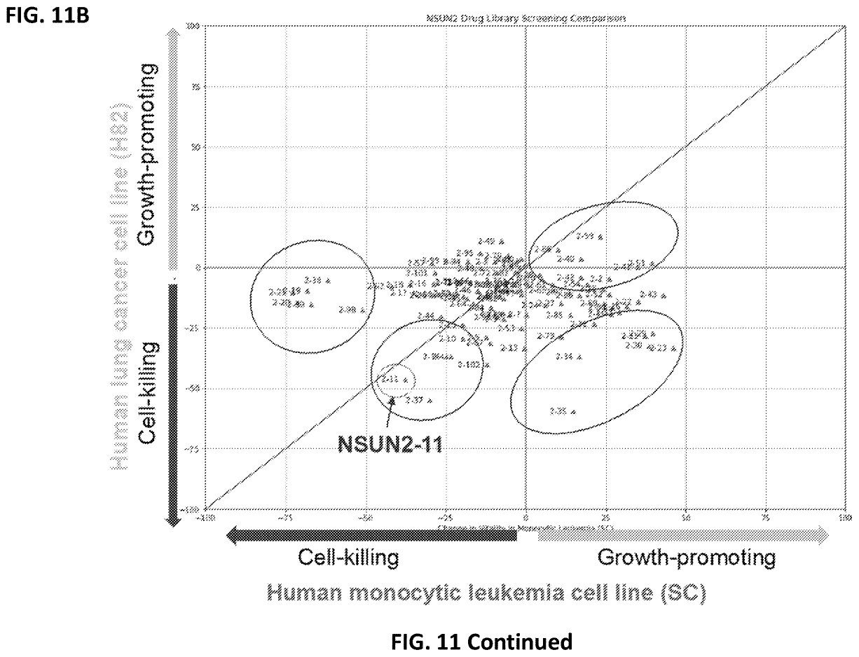

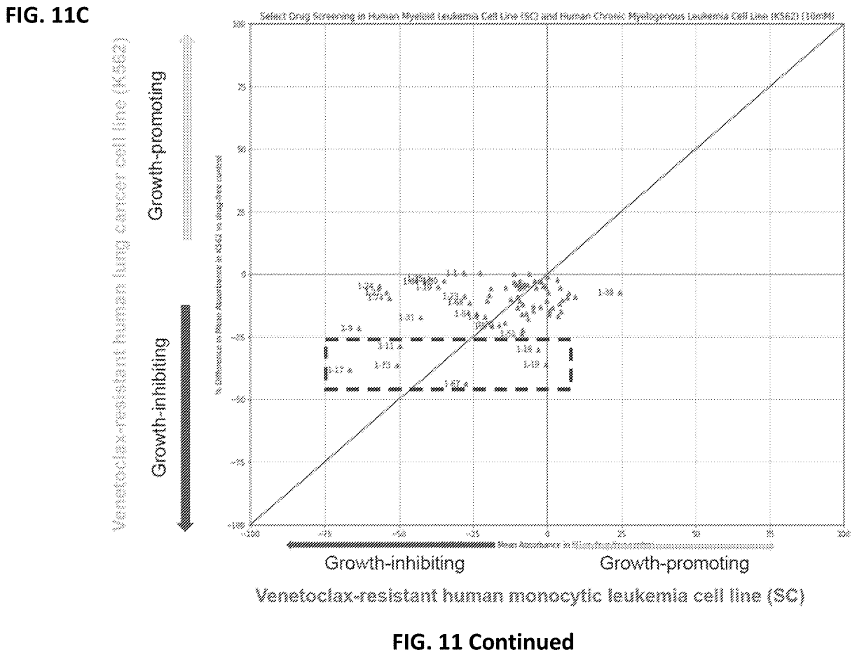

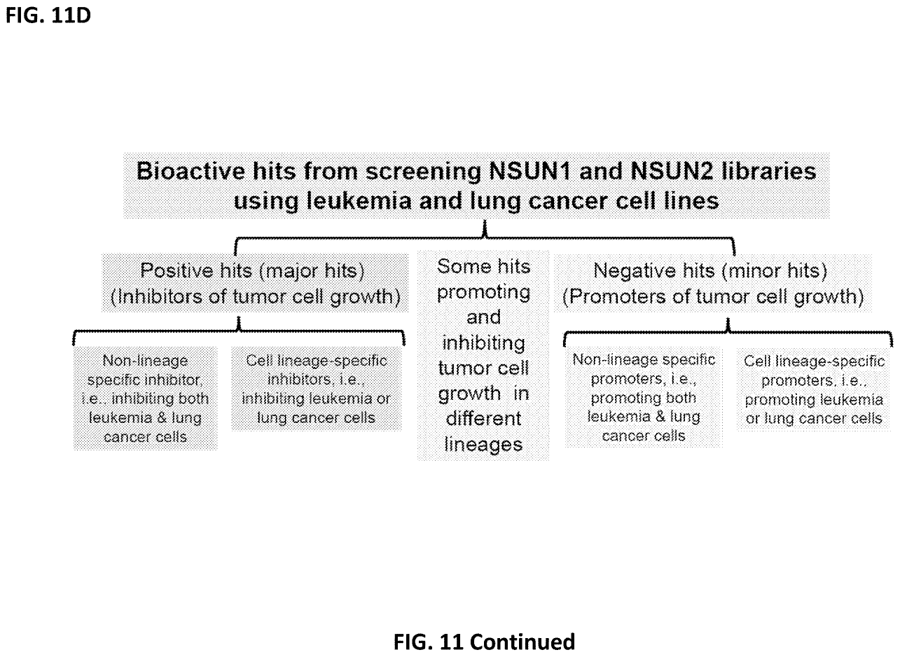

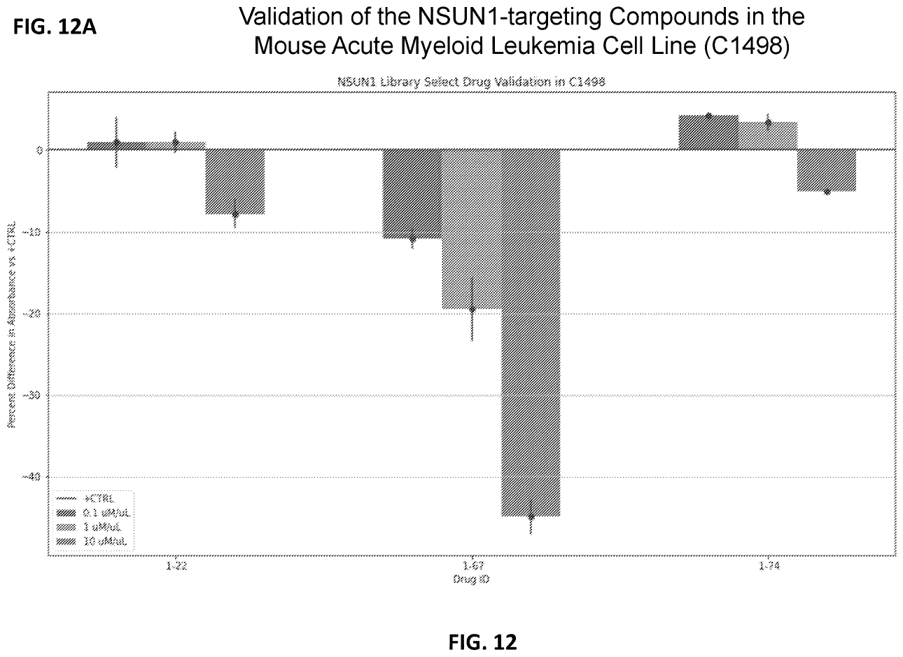

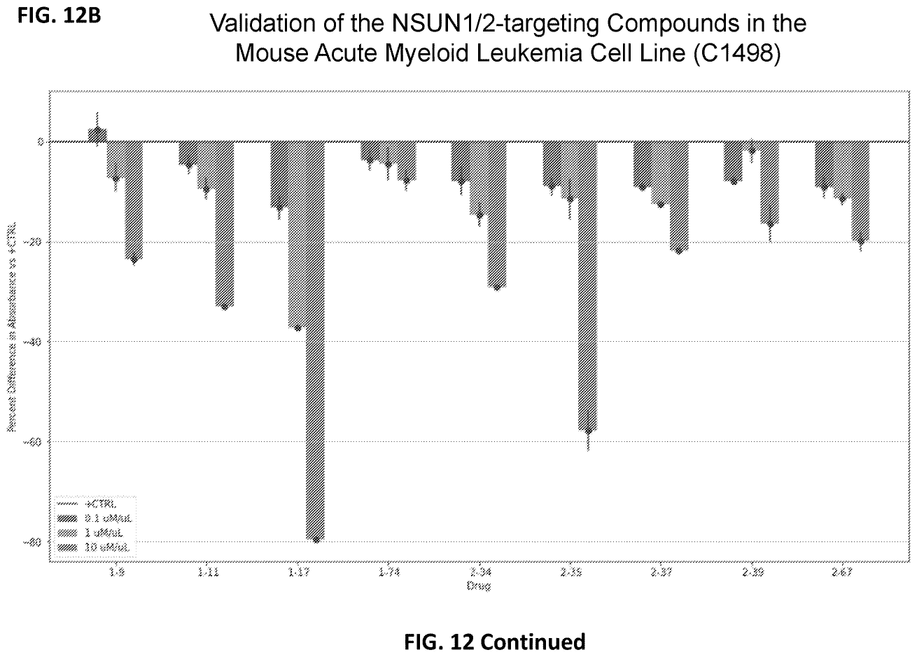

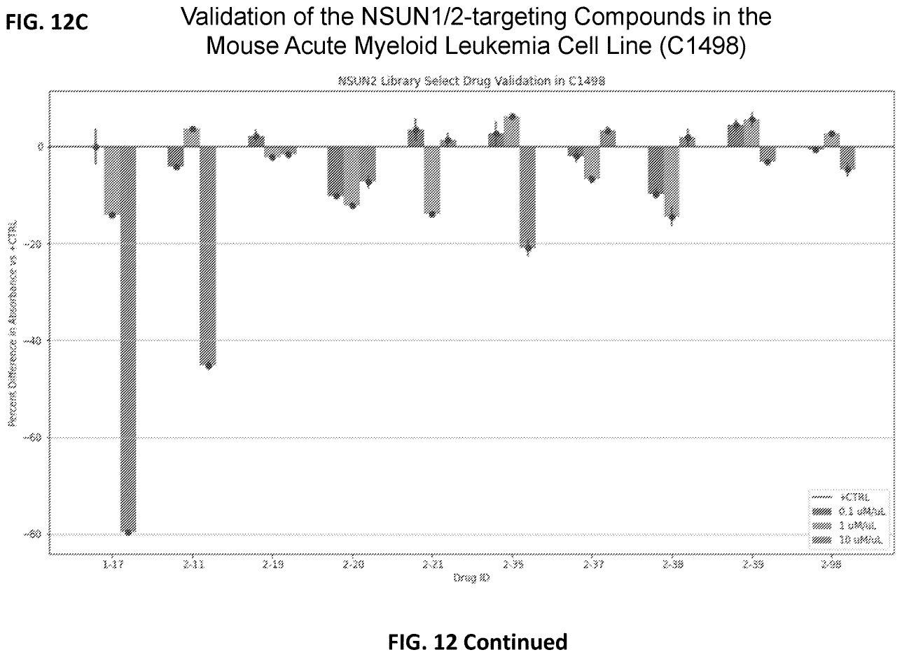

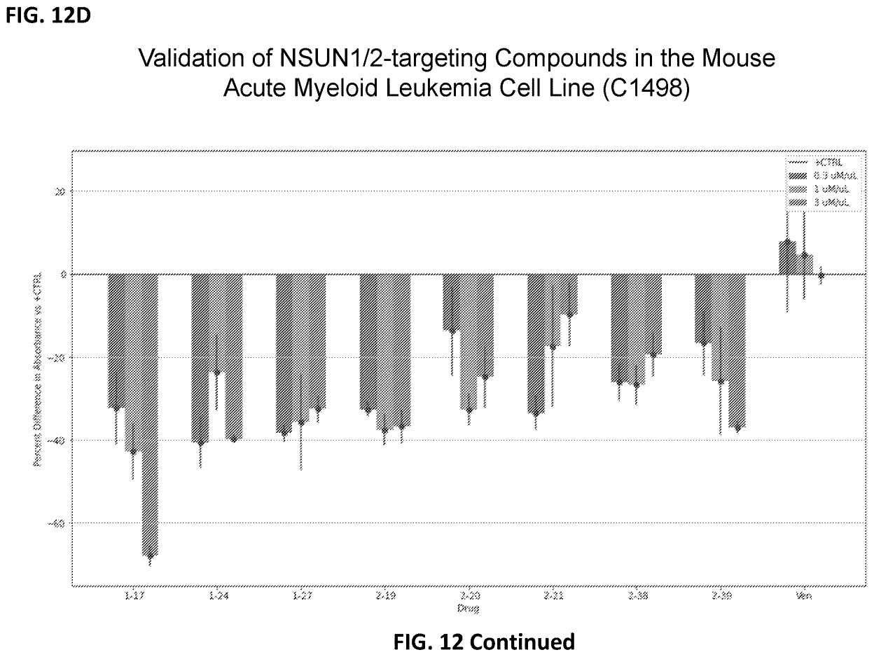

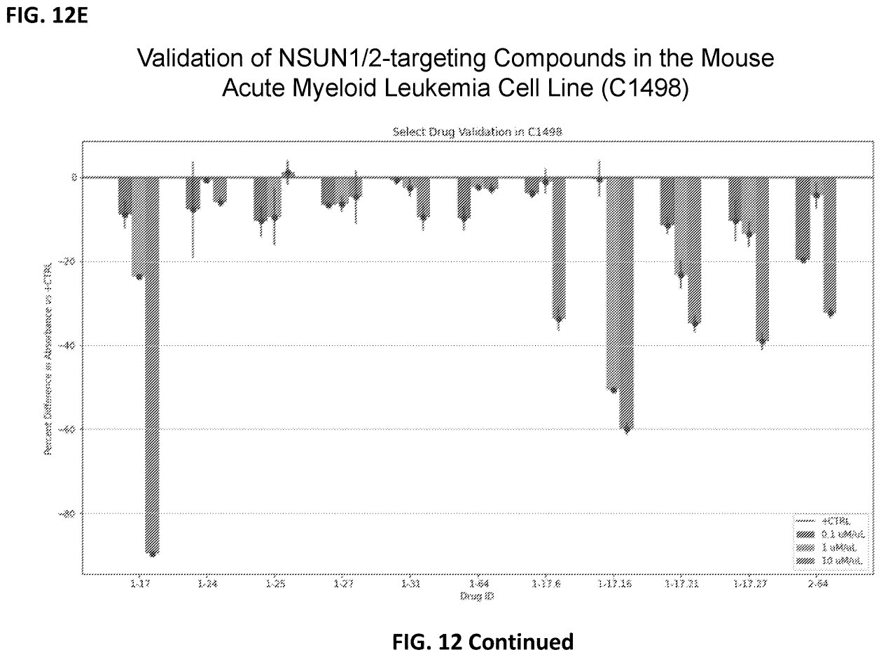

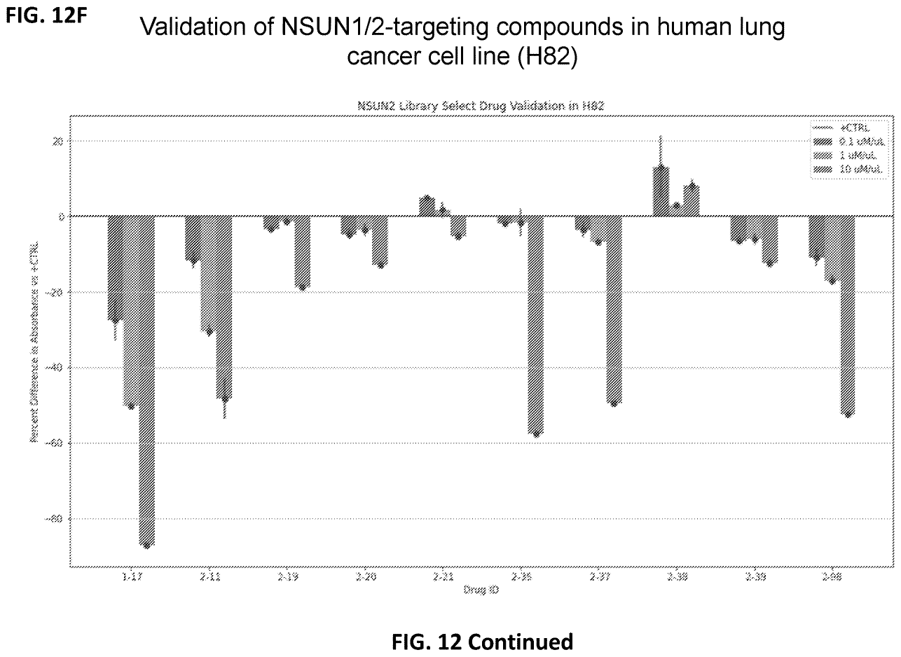

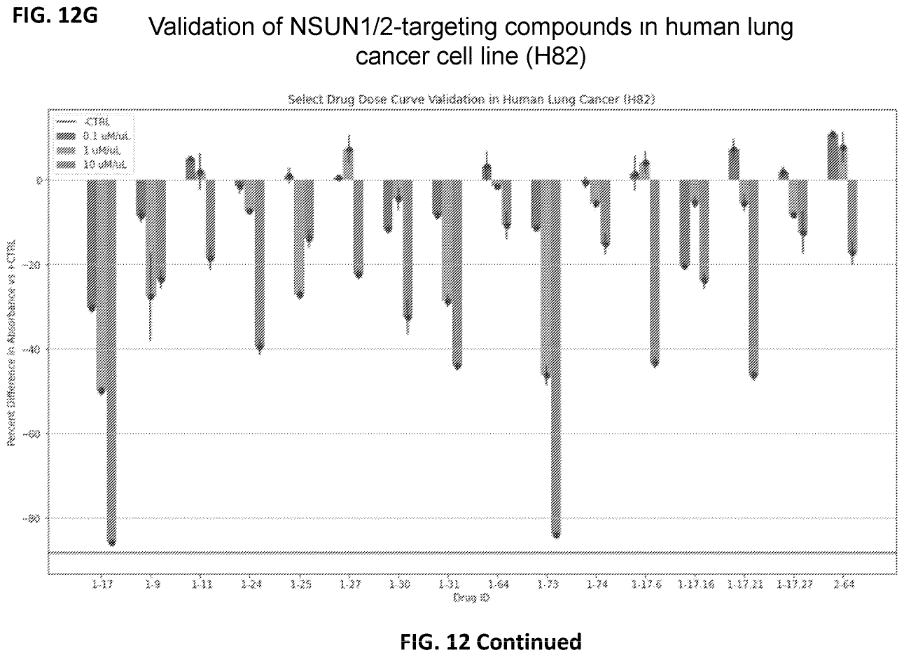

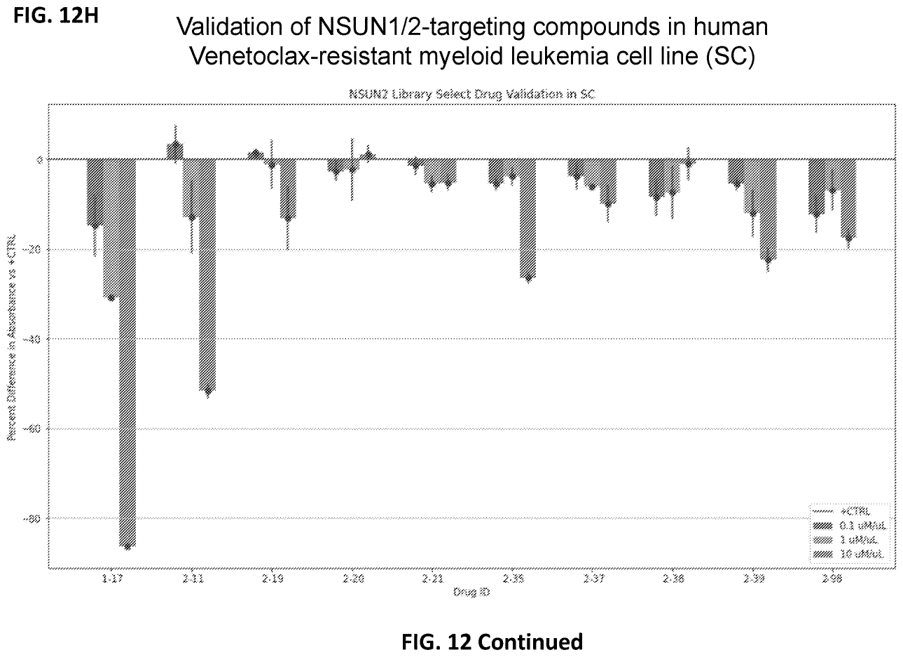

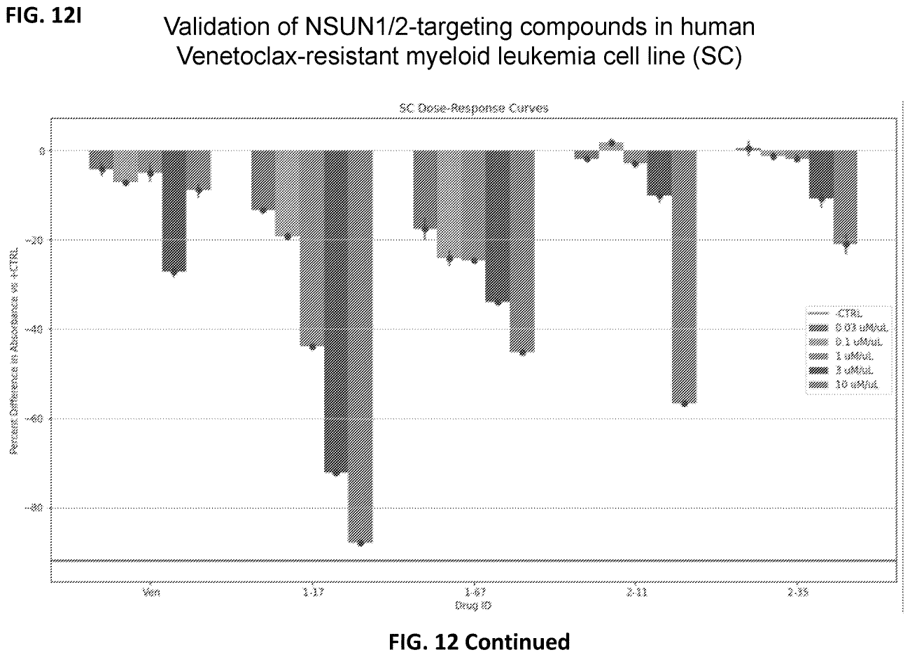

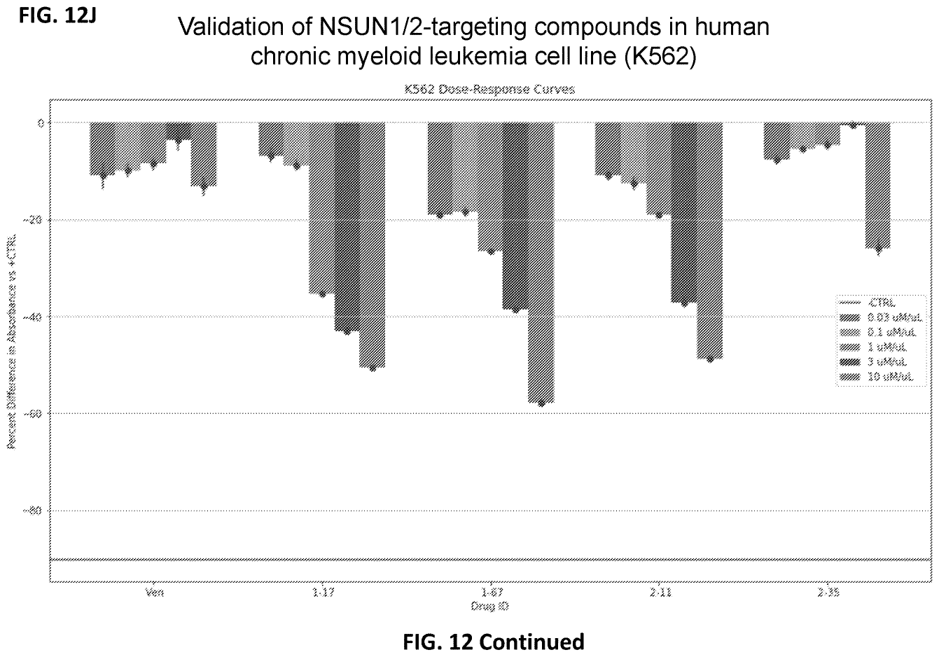

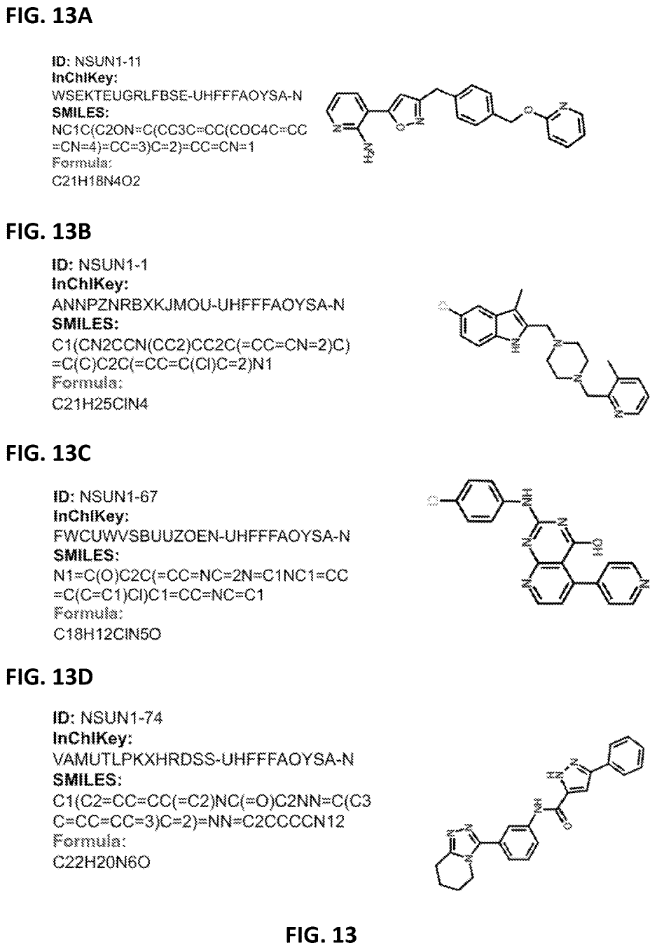

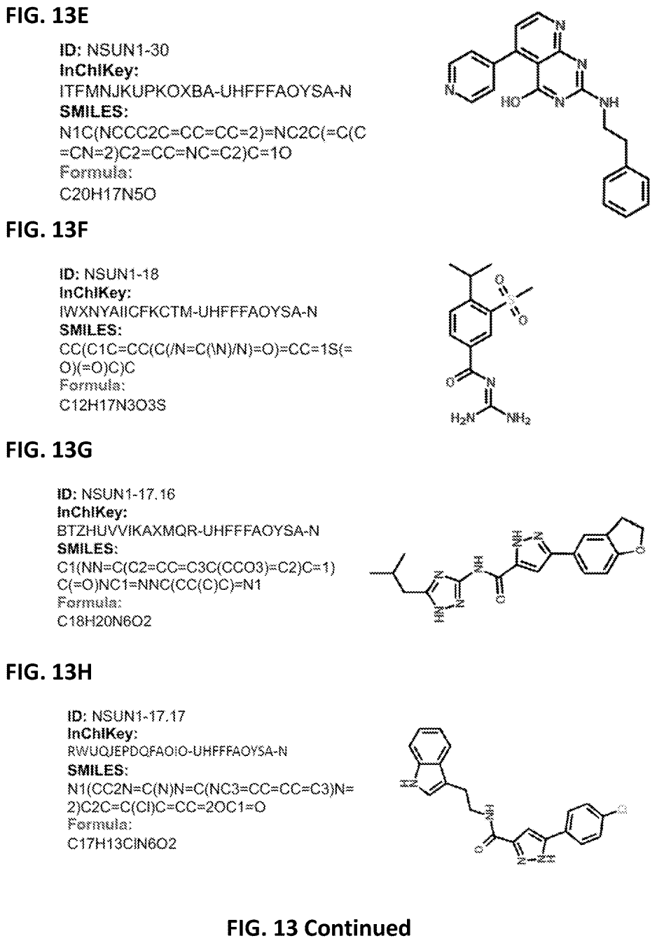

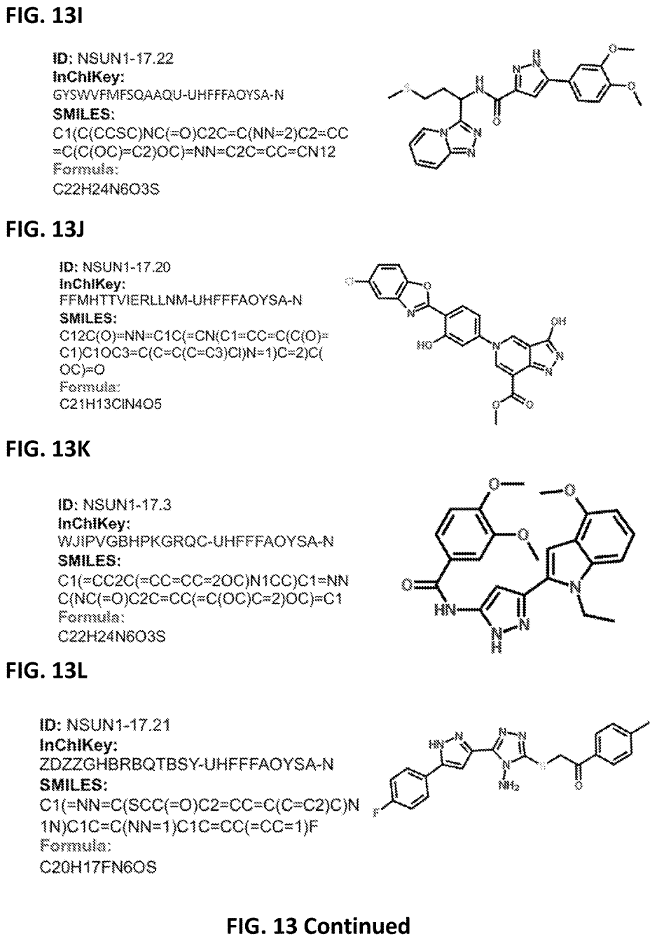

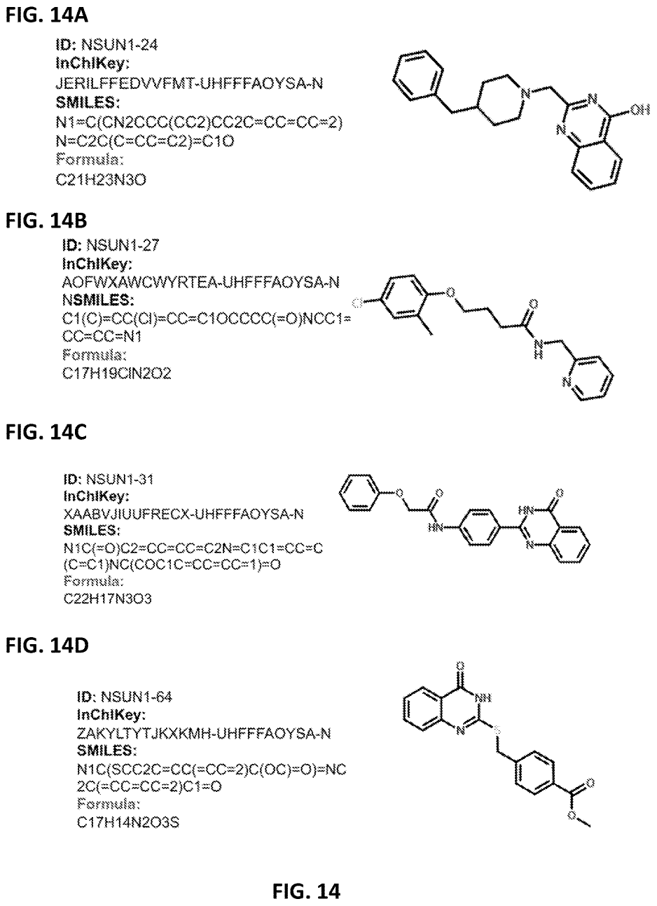

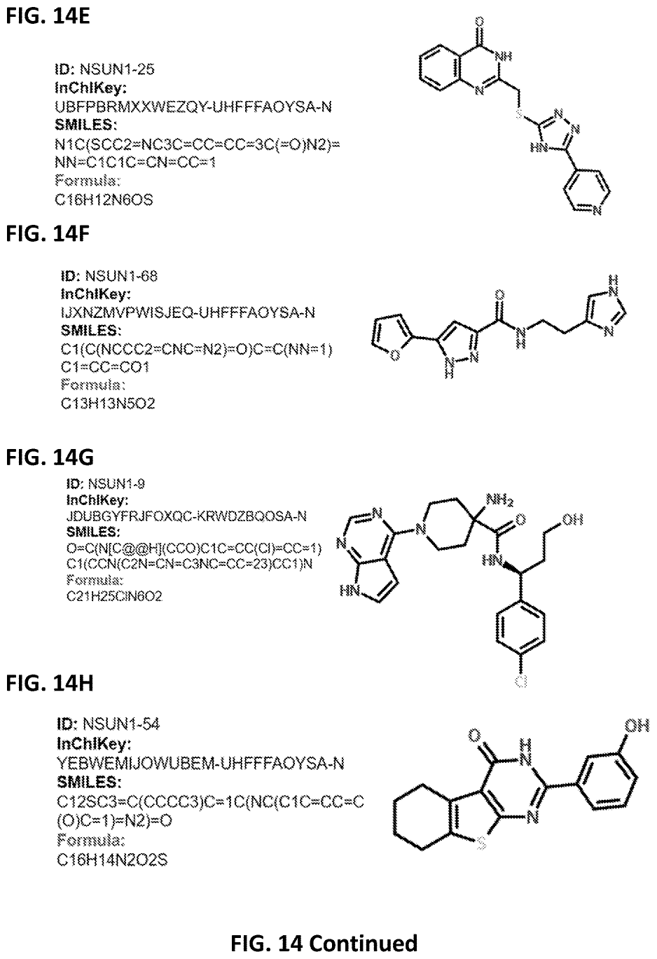

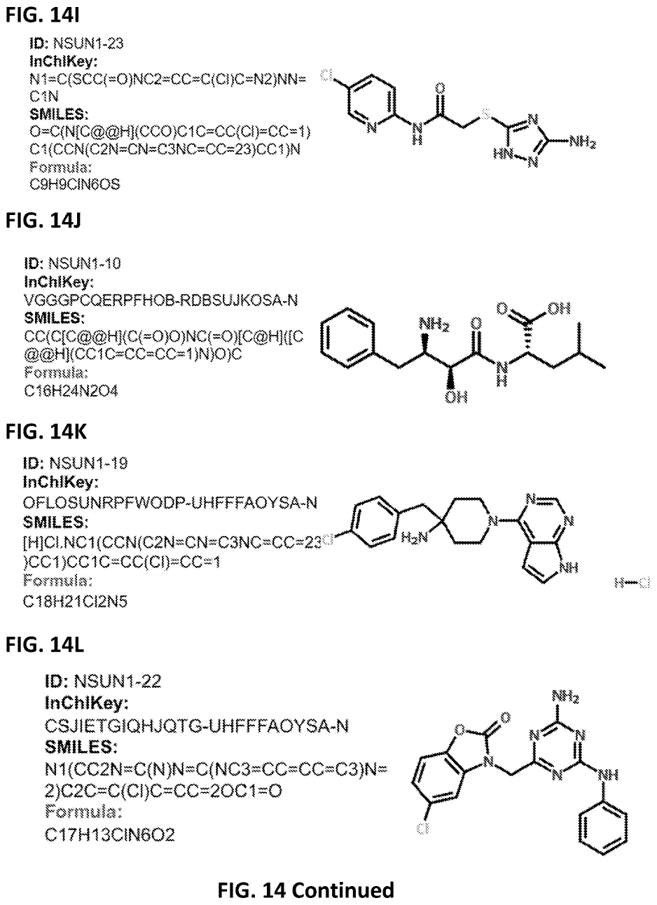

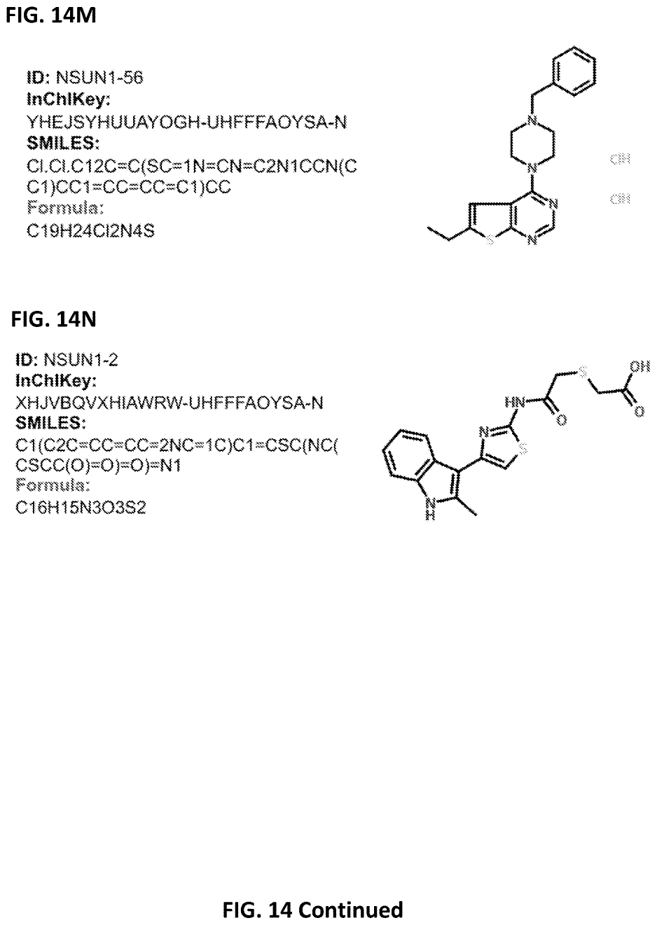

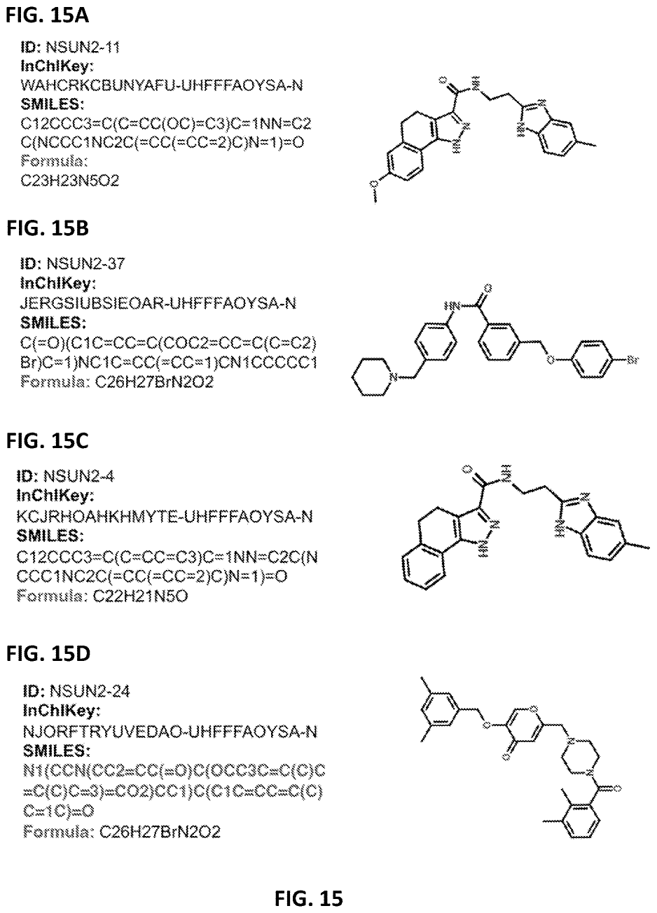

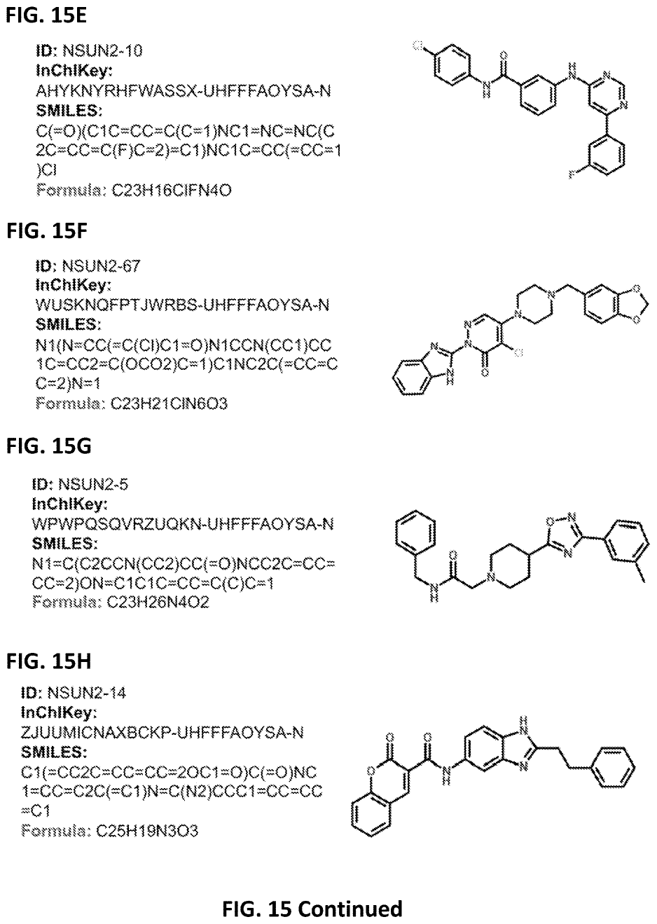

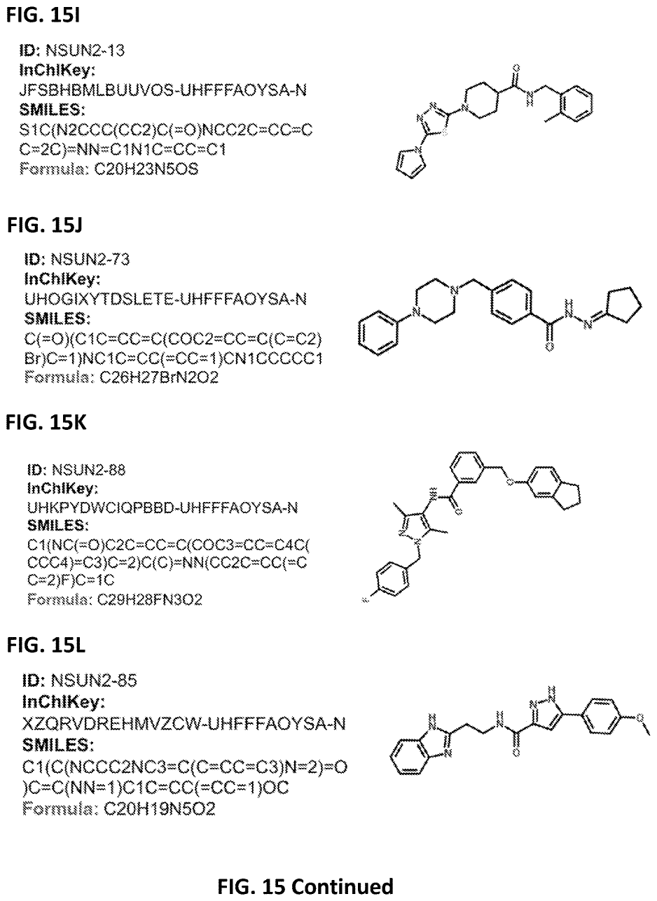

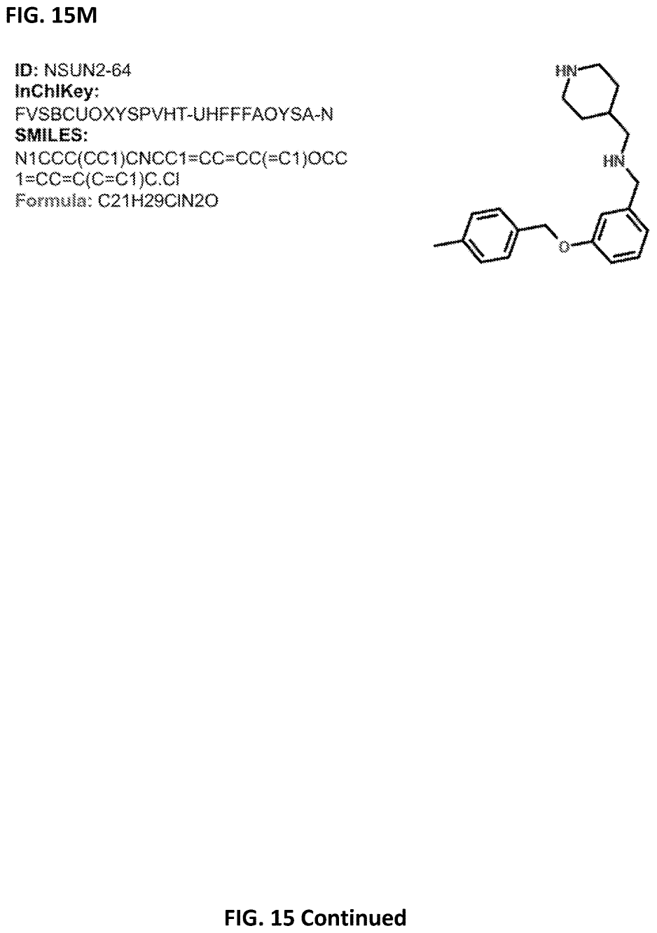

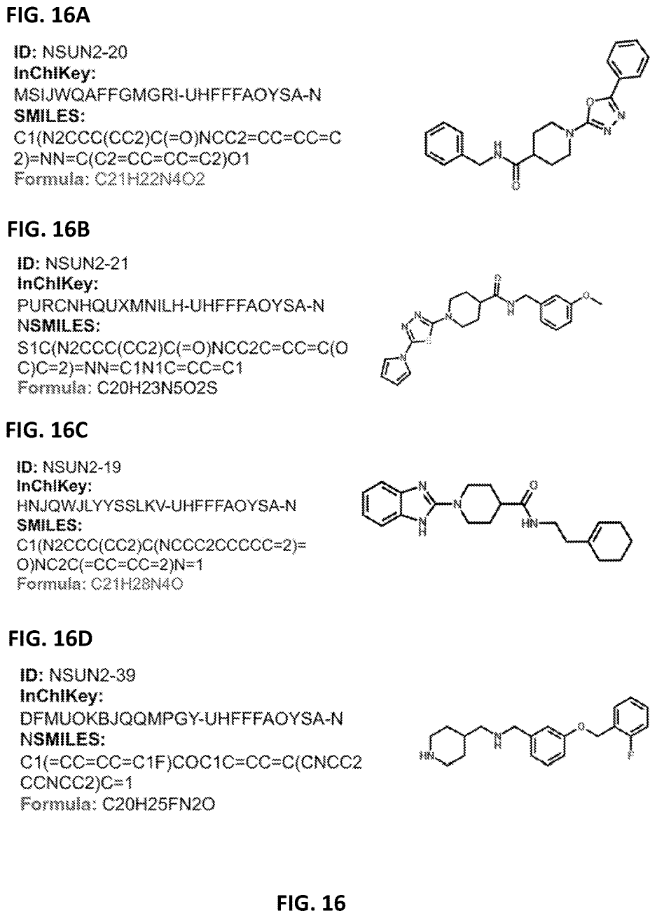

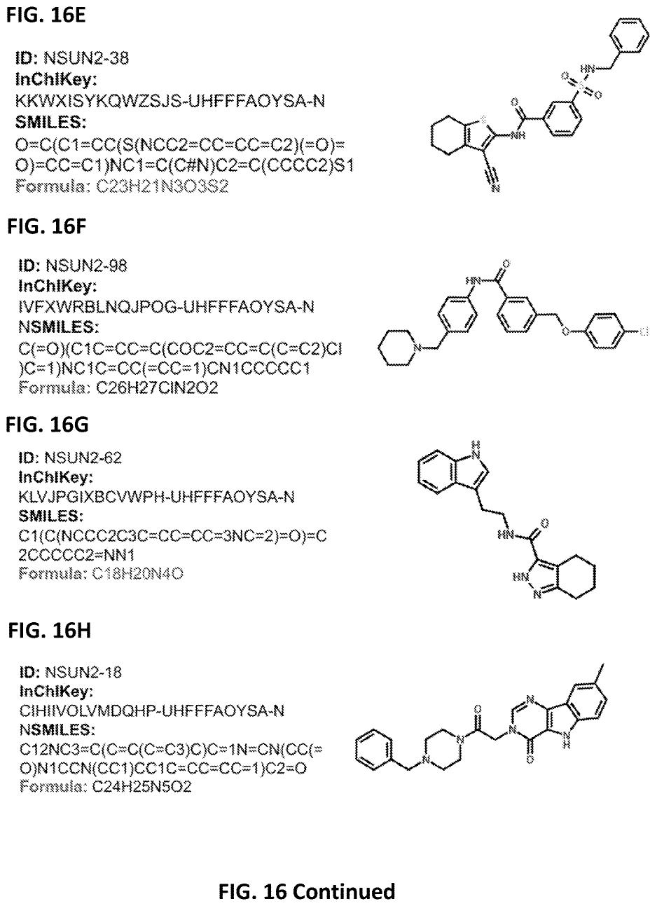

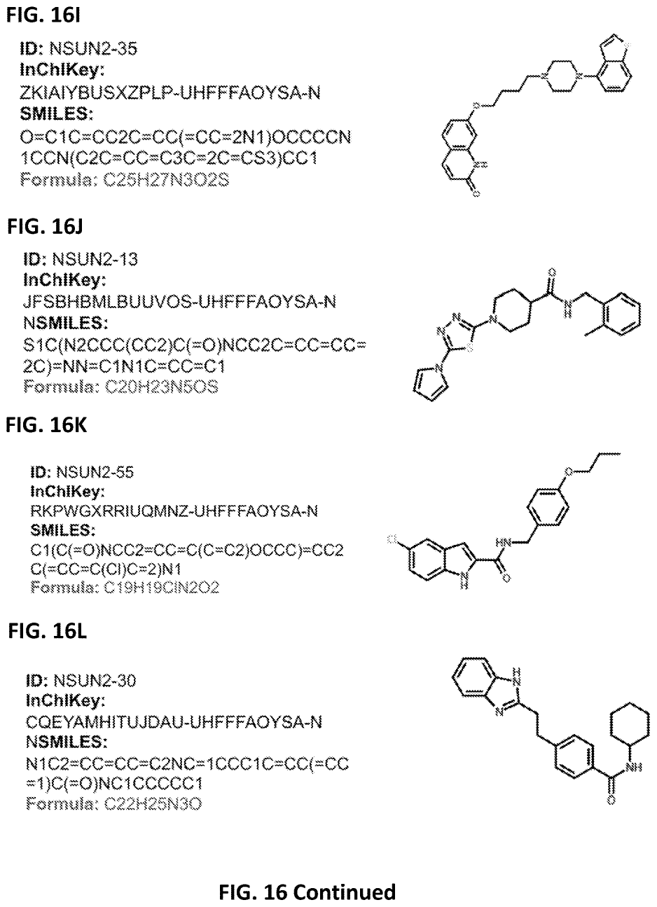

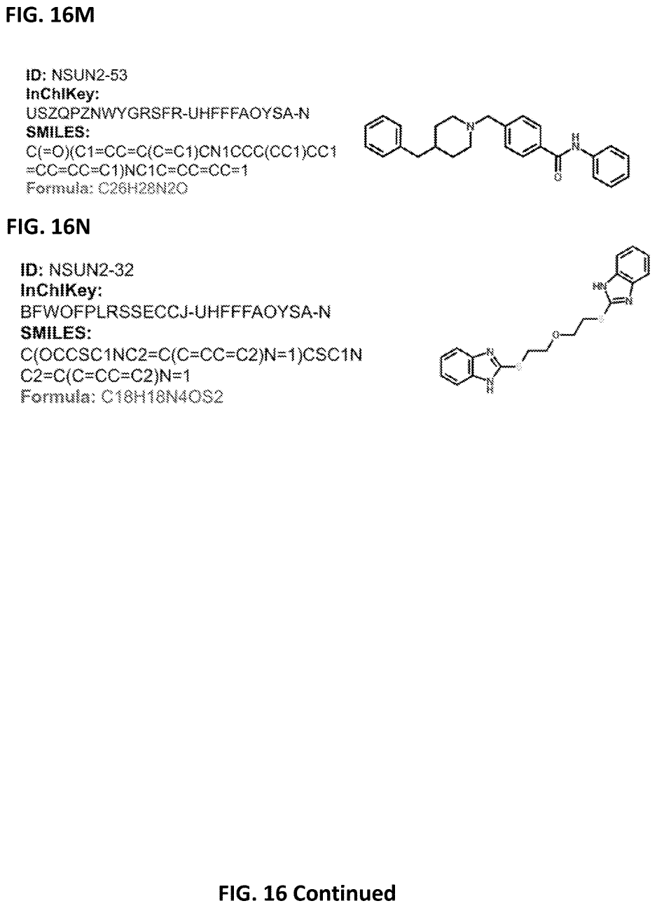

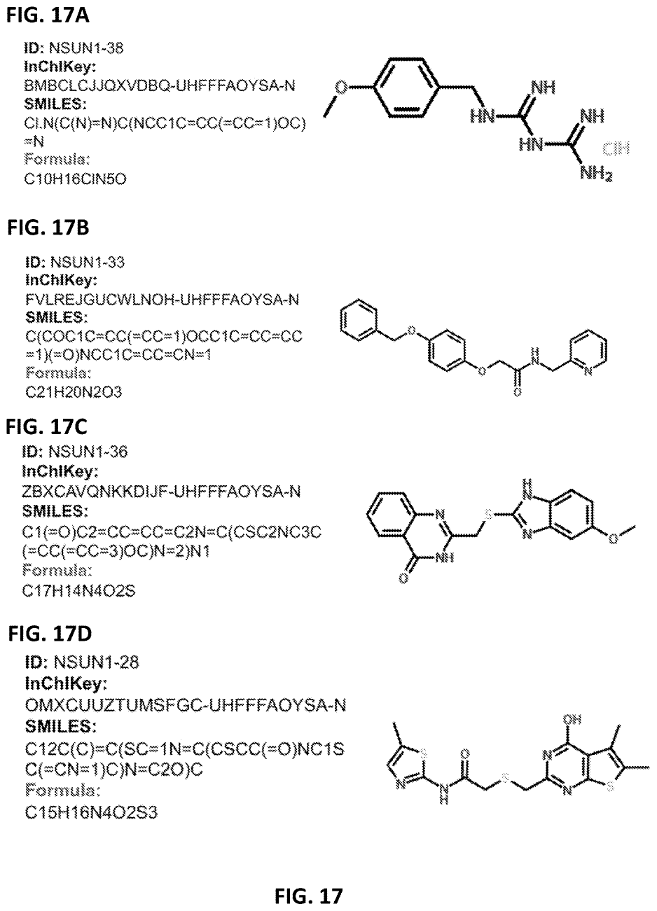

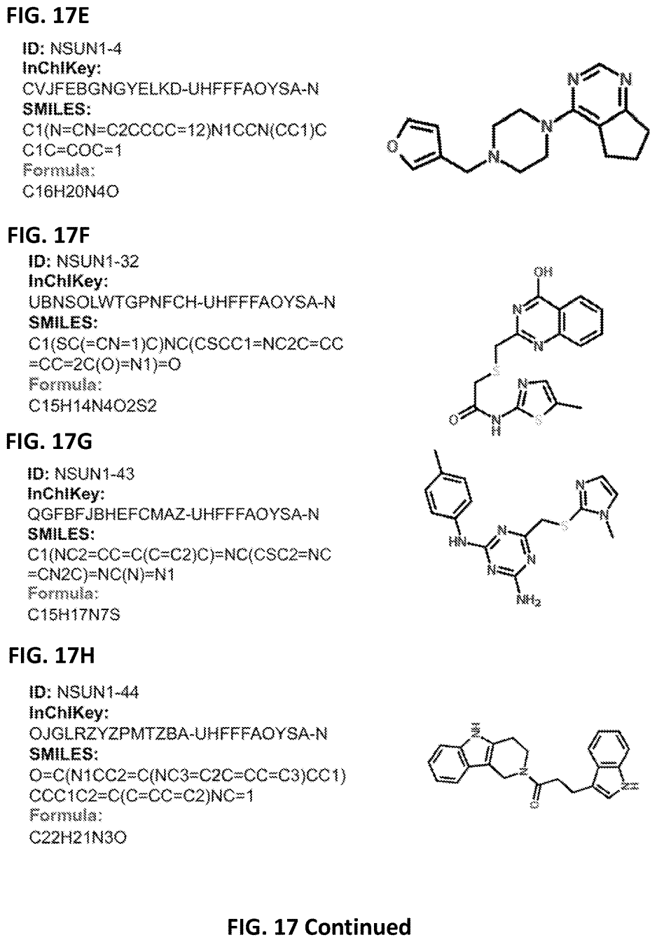

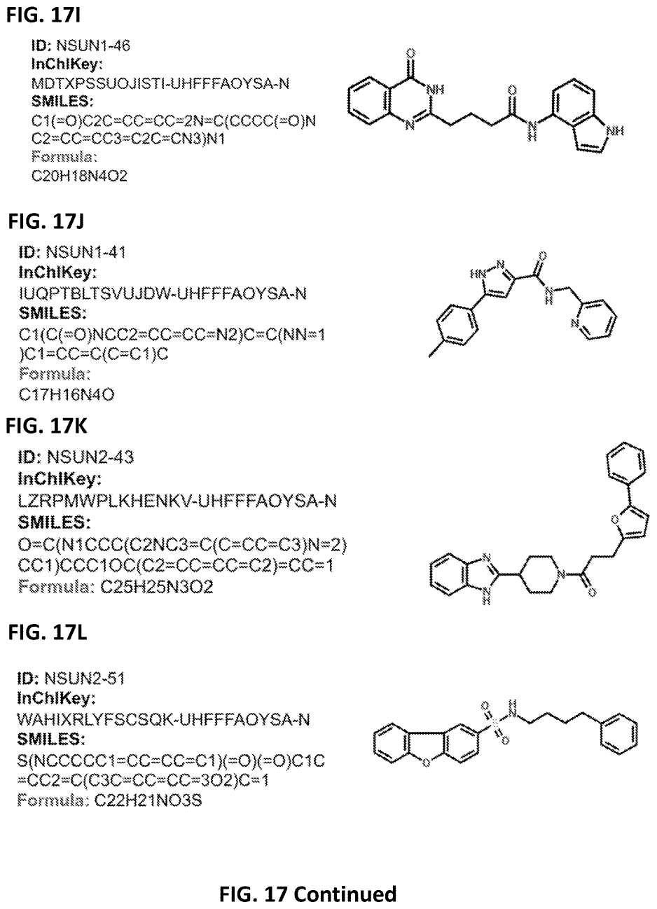

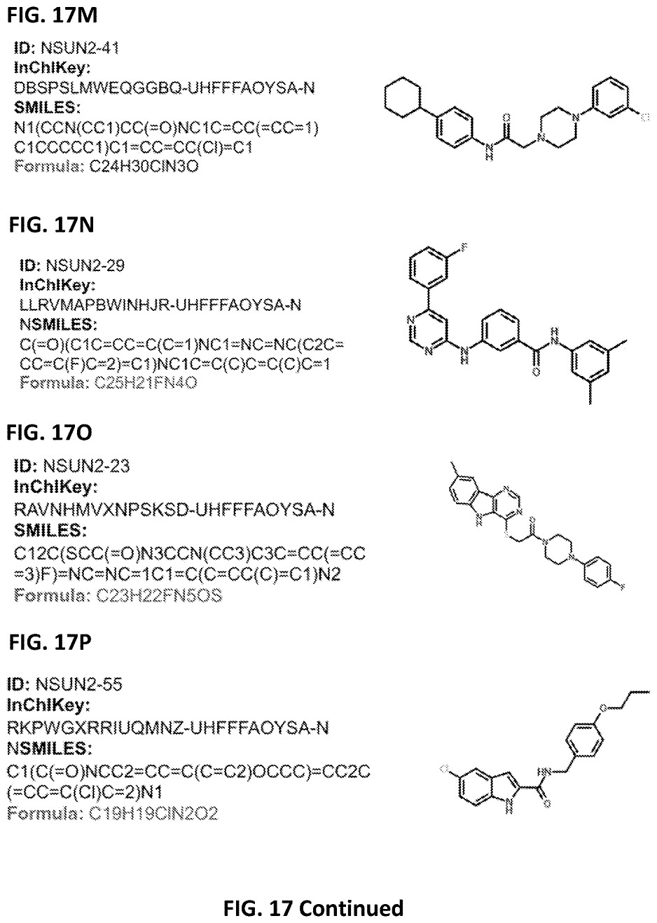

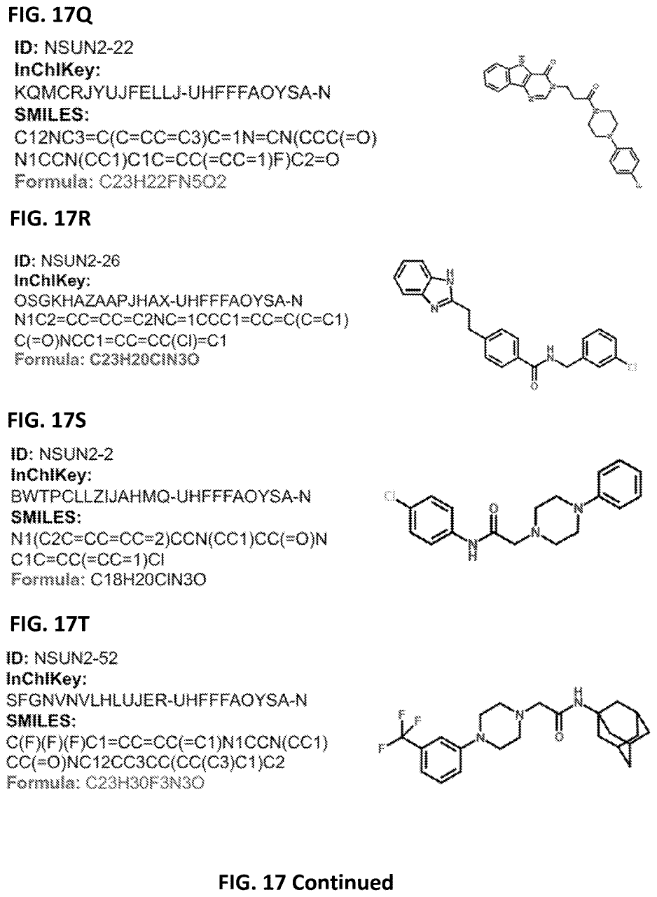

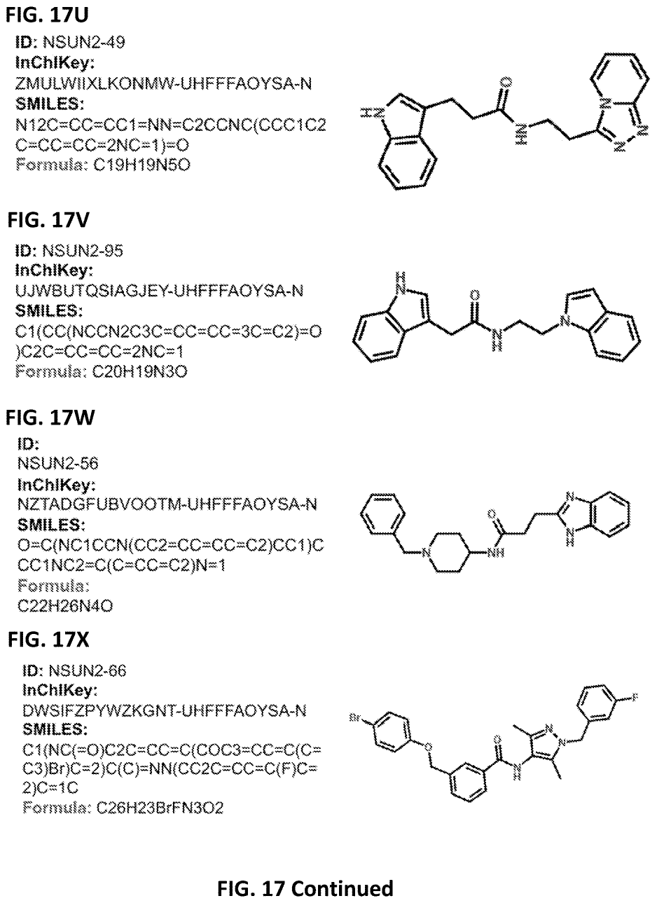

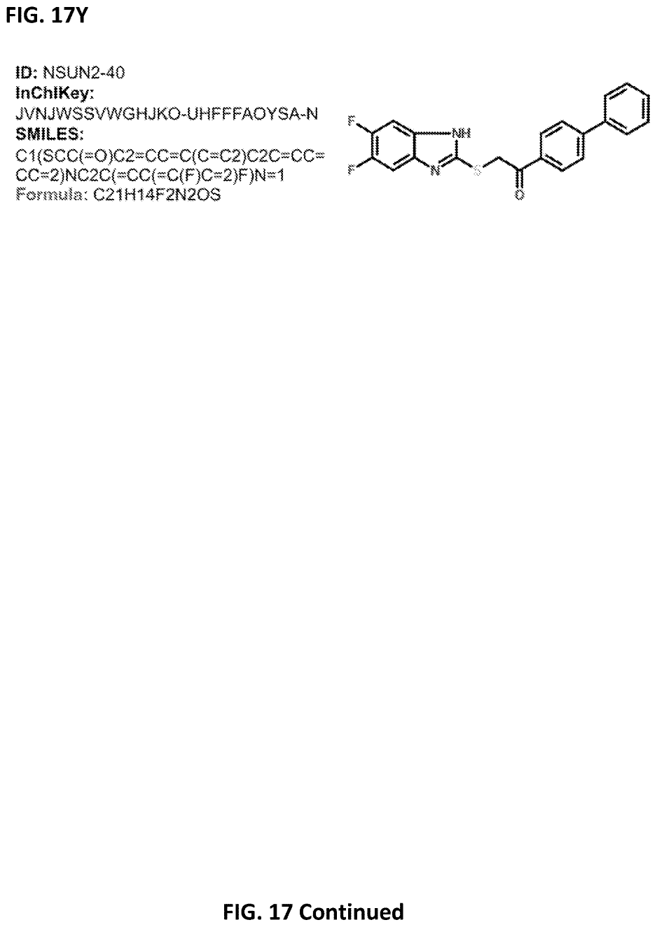

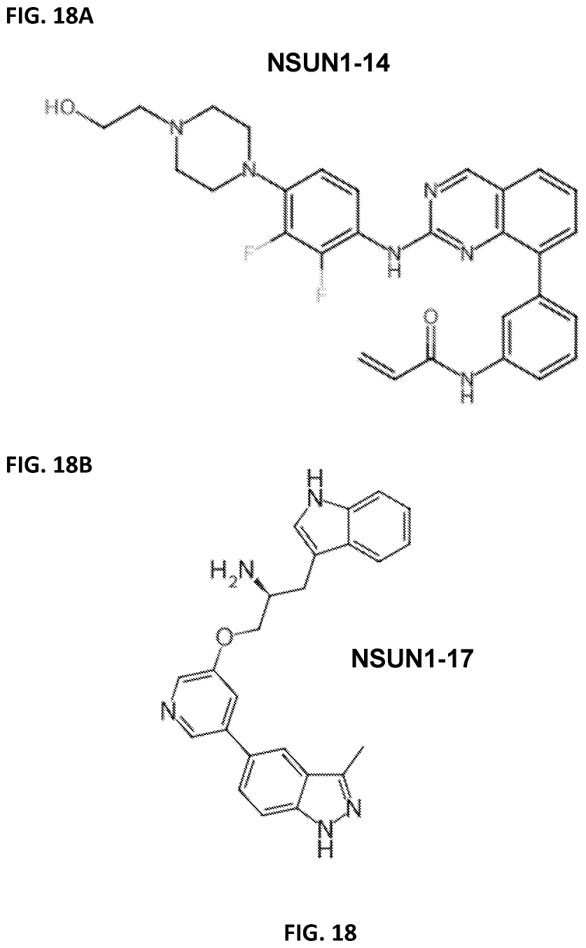

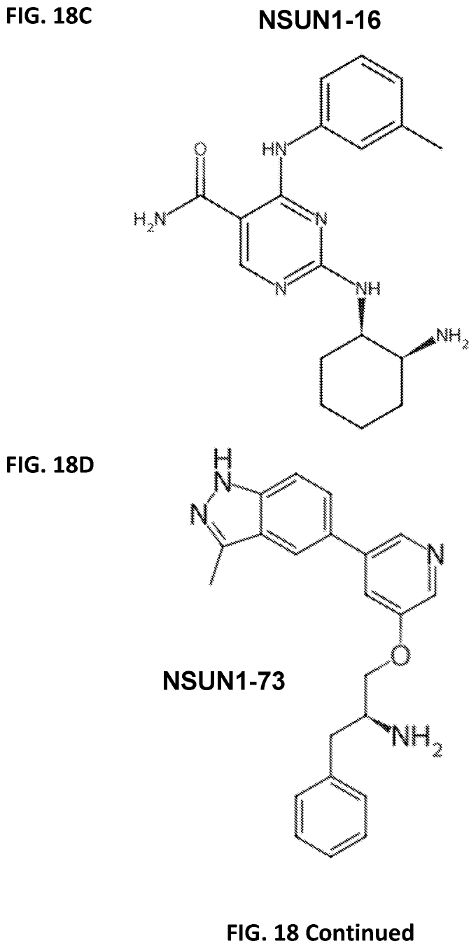

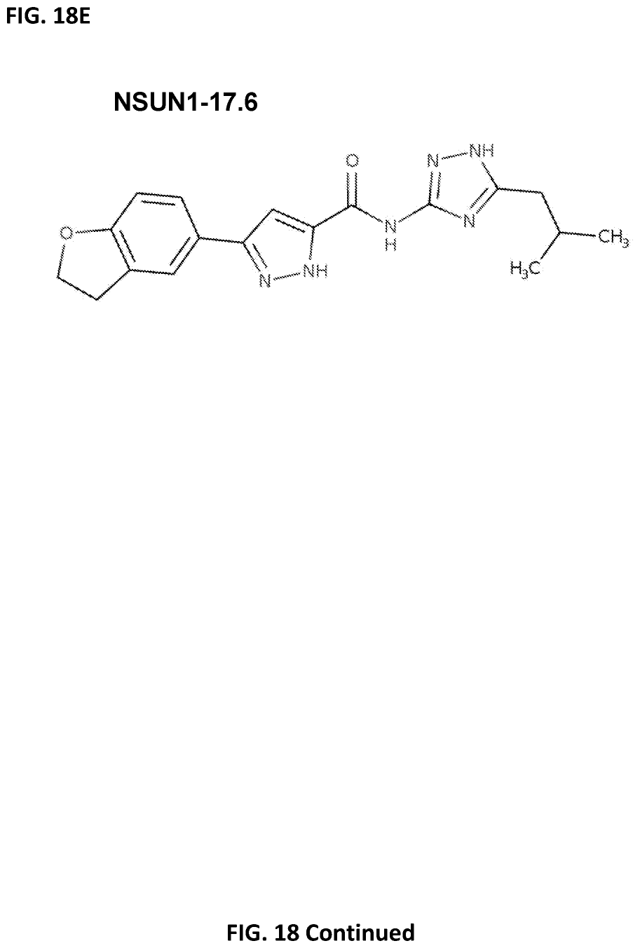

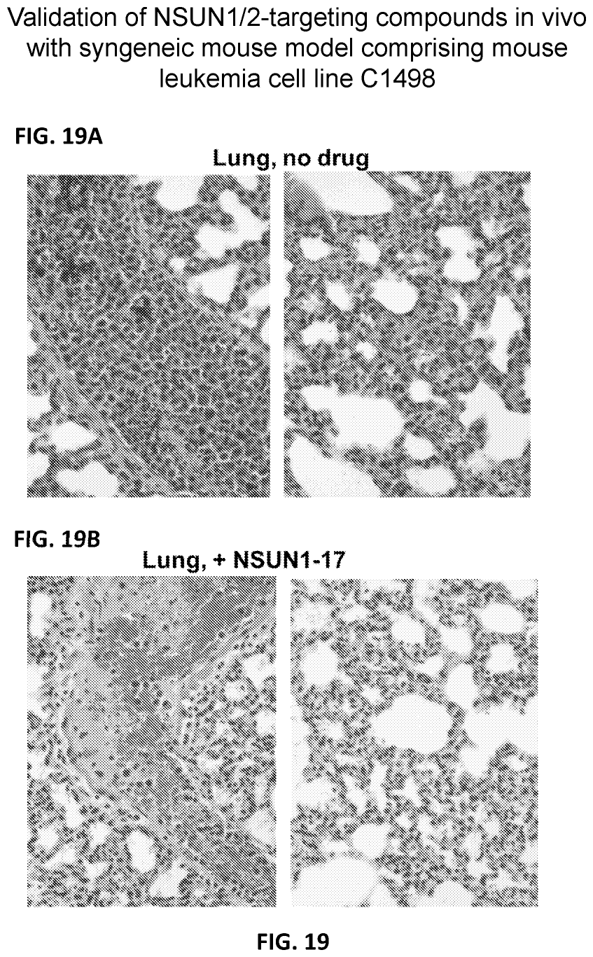

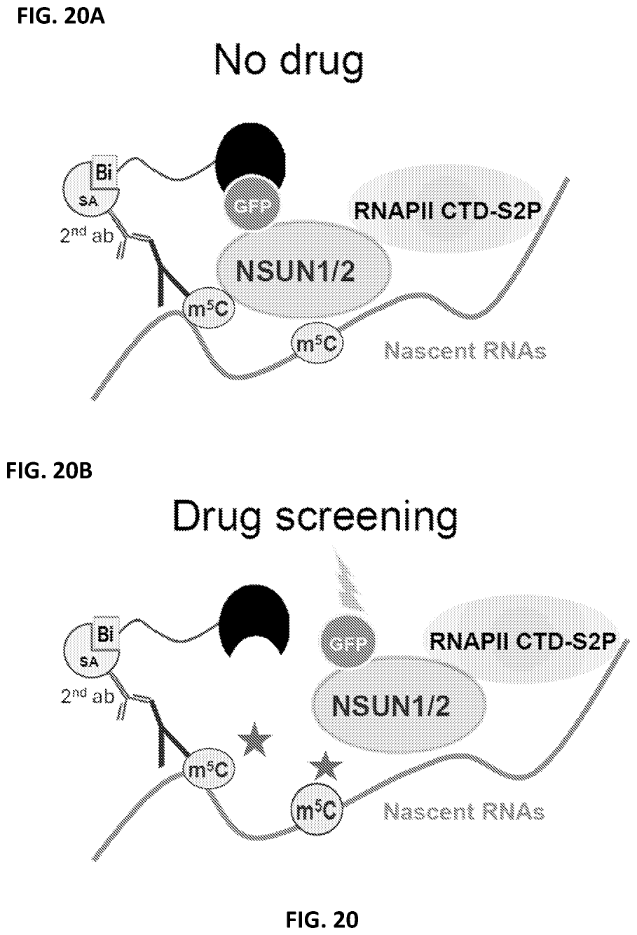

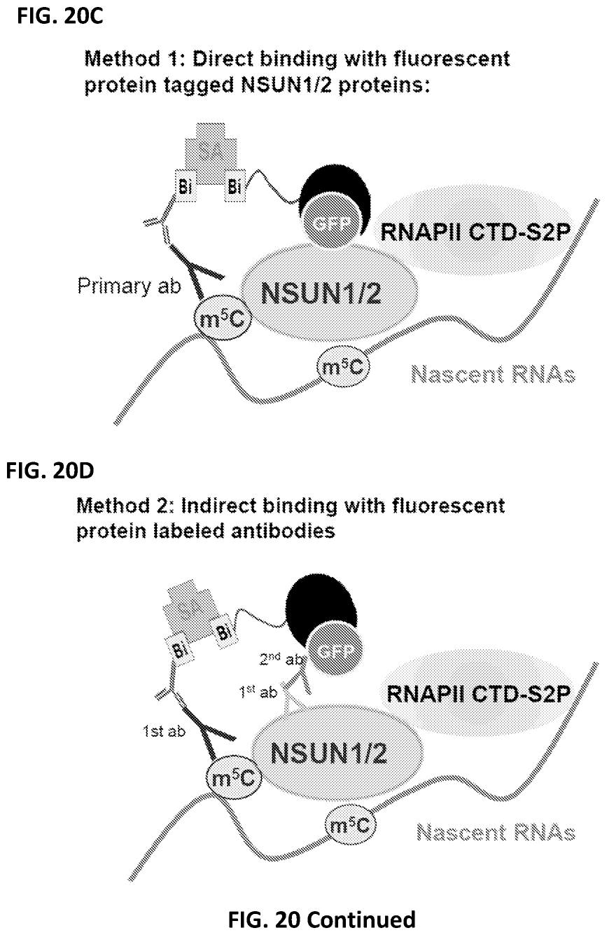

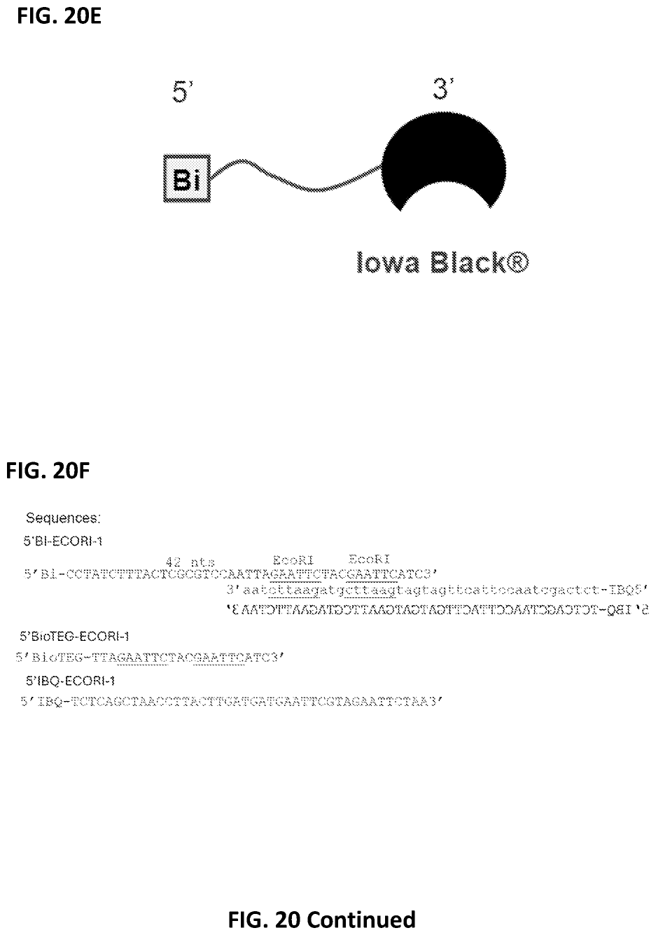

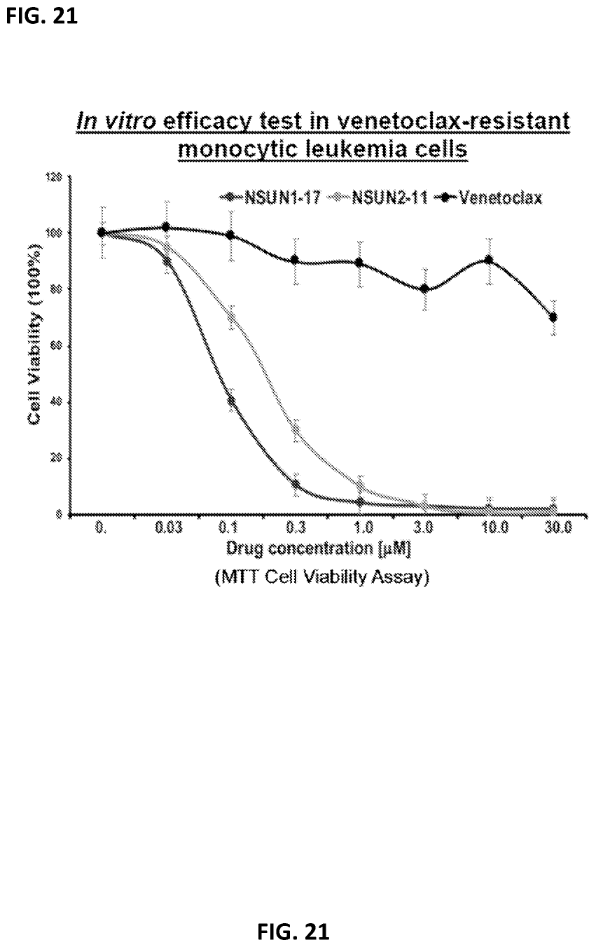

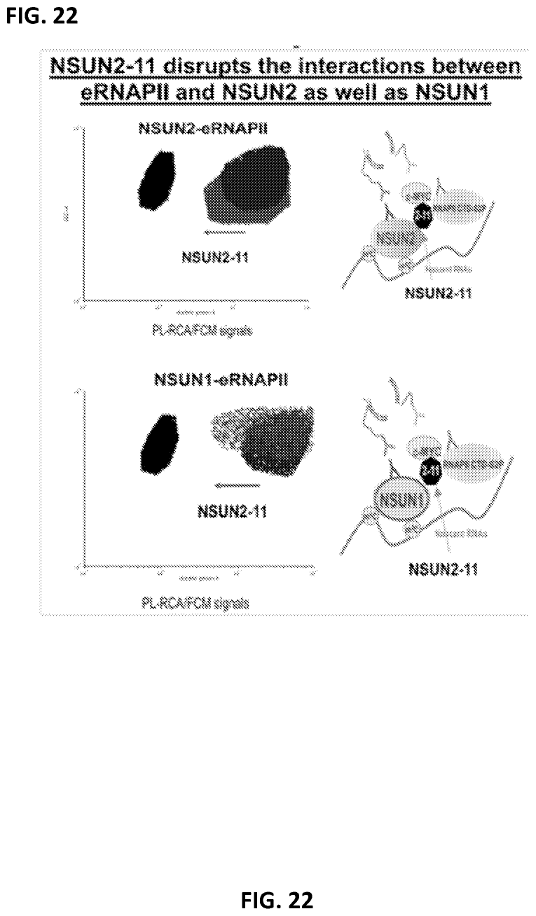

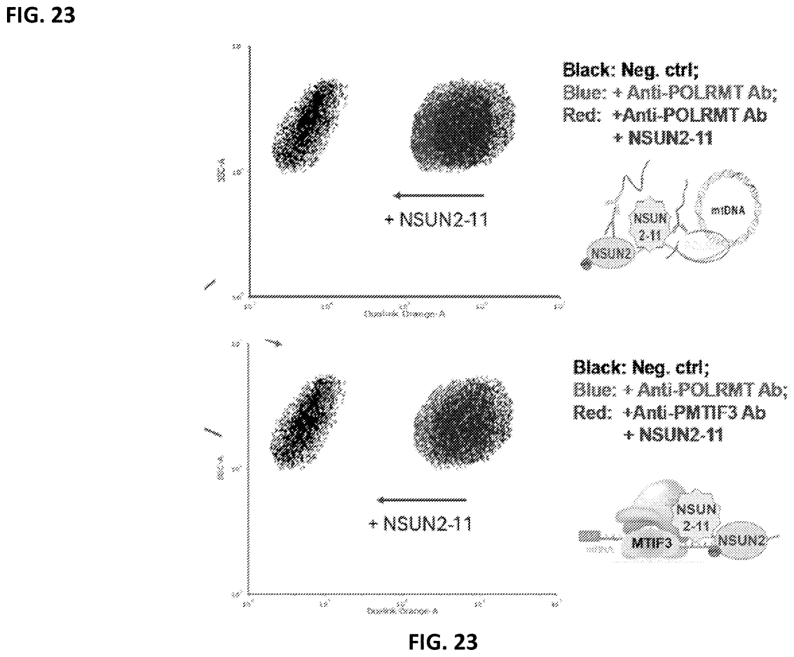

As described herein, the inventors utilized a supercomputer (e.g., the Argonne supercomputer) and ML/AI to predict NSUN1 and NSUN2 protein structures and simulate the interactions between potential ligands/drugs and NSUN1 and NSUN2 proteins (FIG. 5A). The supercomputing and AI/ML identified several potential ligand-binding surfaces/modules in NSUN1 and NSUN2 proteins. As shown in FIG. 5B, provided herein are at least four ligand-binding surfaces or modules for NSUN1 (see FIG. 10), and at least nine ligand-binding surfaces or modules for NSUN2 (see FIG. 10). Novel small-molecule libraries were generated according to those potential ligand-binding surfaces/modules in NSUN1 and NSUN2 proteins FIG. 5C. Provided herein are small-molecules that successfully bound to NSUN1 and/or NSUN2 and inhibited drug-resistant cancer cell proliferation. In certain aspects, also provided herein are NSUN1 and/or NSUN2 binding small-molecules that can promote drug-resistant cancer cell proliferation, where in some aspects, such information can be useful for discerning the relevant importance of select NSUN1 and/or NSUN2 binding modules. For example, at least the identified compounds NSUN1-11 and NSUN1-17 were found to inhibit drug (venetoclax)-resistant leukemia cells. Redocking the compounds to NSUN1 protein demonstrated that both NSUN1-11 and NSUN1-17 compounds bind to the A-460 surface/module (FIG. 5E).

In some aspects, provided herein are experimental results from procedures comprising computational simulation of NSUN1/2 protein structures, and the design of the NSUN1/2-targeting compound libraries. In some aspects, supercomputing/AI design of NSUN1/2-targeting libraries is performed. In some aspects, supercomputing/AI design comprises: 1) chemical compound structures documented in various chemical compound database are uploaded onto the supercomputer and normalized for supercomputing and AI/ML simulation; 2) the structures of those chemical compounds are filtered through ML/AI convolutional neural network; 3) the supercomputer/AI simulates the massive number of potential interactions between drugs/compounds and targeting proteins to score strong interactions between the predicted ligand-binding surfaces/modules in NSUN1 and NSUN2 proteins with the tested drugs/compounds; 4) those compounds with the highest binding potentials (scores) predicted by the supercomputing/AI simulation process are used to generate the NSUN1/2-targeting drug/compound libraries, which include drugs/compounds with known bioactivities, and compounds with to-be-determined bioactivities.

In some aspects, following supercomputer/AI mediated identification of putative NSUN1/2-targeting compounds, functional screening of the NSUN1/2-targeting compound libraries are performed. In the past, some of the inventors research has focused on exploring the mechanisms underlying the lineage-associated drug selectivity and resistance, and developing new strategies and technologies to predict and overcome drug resistance in hematologic malignancies. The inventors efforts led to the development of RNA epigenetics driven novel technologies and therapeutic targets. For example the inventors discovery of RNA cytosine methyltransferases (RCMTs) NSUN1 and NSUN2-mediated drug-resistant active chromatin structures (ACS) in leukemia cells. In the inventors previous study, Cheng, J. X. et al., Nature Communications 2018 (which is incorporated herein in its entirety by reference), the relationship between 5-AZA resistance and eRNAPII complexes was investigated. The inventors compared the 5-AZA resistant M2AR and SCAR cells against 5-AZA sensitive OCI-M2 and SC cells. After screening various RCMTs, the inventors identified NSUN1 as a unique RCMT that binds eRNAPII (CTD-S2P), only in the 5-AZA-resistant M2AR cells, but not in the original 5-AZA-sensitive OCI-M2 cells.

The interactions between eRNAP-II (CTD-S2P) and NSUN1 as well as CTD-S5P are resistant to 5-AZA. In contrast, the markedly increased interactions between eRNAPII (CTD-S2P) and NSUN3 as well as DNMT2 are highly sensitive to 5-AZA. BRD4 is a member of the BET (bromodomain and extra terminal domain) protein family and can function as an atypical kinase to phosphorylate the eRNAPII. In the azacitidine-resistant M2AR cells there is an 80-120-fold increase in BRD4-associated eRNAPII (CTD-S2P) compared to azacitidine-sensitive leukemia cells, as well as a unique BRD4-NSUN1 interaction. The interactions between BRD4 and eRNAPII (CTD-S2P) as well as NSUN1 were insensitive to the molecule JQ1 that can block BRD4 binding to acetylated lysine residues of histones to inhibit BRD4-mediated transcriptional activation.

To dissect the mechanisms underlying drug (azacitidine) resistance and its lineage association, the inventors demonstrated the roles of individual RCMTs in the regulation of drug (azacitidine)-resistant leukemia cells, through knocked down of the expression of RCMTs by specific siRNAs, the results demonstrated a general inhibition effect on the growth of the MDS/AML cells. However, the 5-AZA resistant SCAR cells were more sensitive to the downregulation of NSUN1 and NSUN2 expression than the 5-AZA-sensitive SC cells. Knockdown of NSUN1 expression re-sensitized the 5-AZA-resistant SCAR cells to low concentrations of 5-AZA and induced growth inhibition. The data of NSUN2 knockdown were similar to that of NSUN1. These data demonstrated the importance of NSUN1 and NSUN2 in determining azacitidine resistance in leukemia cells.

In the same publication (Cheng, J. X. et al., Nature Communications 2018), the inventors compared the RCMTs that mediated ACS in the azacitidine (a.k.a. 5-AZA)-resistant M2AR cells and SCAR cells compared to the original 5-AZA-sensitive OCI-M2 and SC cells. 5-ethynyl uridine (EU)-clicking chemistry confocal microscopy (EC-CM) illustrated the subcellular localization of NSUN1, BRD4 and eRNAPII (RNA-pol-II CTDS2P) and their relationship to nascent RNA in M2 and M2AR cells. OCI-M2 cells had a weaker and more diffused pattern of NSUN1 and BRD4. In contrast, M2AR cells showed stronger expression of NSUN1 and BRD4, and these molecules were are co-localized to form distinct granular particles with intensified peripheral zone stain at nascent RNAs in M2AR cells BRD4. Similar patterns of subcellular localization and co-localization of NSUN1, eRNAPII and nascent RNAs were observed in OCI-M2 and M2AR cells as well. In situ proximity ligation rolling circle amplification (PL-RCA) was performed to further confirm the co-localization of NSUN1 with BRD4 and eRNAPII. These experiments demonstrated a marked increase in the PL-RCA signals with the pairs of antibodies against NSUN1 and BRD4 as well as NSUN1 and RNA-pol-II CTD-S2P in M2AR cells BRD4, compared to those in OCI-M2 cells. A similar pattern of the PL-RCA signals of co-localization of NSUN1 with BRD4 and eRNAPII was observed with the same pairs of antibodies in SCAR vs. SC cells. These data demonstrated the presence of distinct NSUN1-/eRNAPII/BRD4-associated ACS at nascent RNA in the drug (azacitidine)-resistant leukemia cells.

In the same publication (Cheng, J. X. et al., Nature Communications 2018), the inventors investigated clinical AML/MDS specimens. PLRCA-CM was performed in the azacitidine-sensitive and -resistant MDS/AML clinical bone marrow (BM) specimens (n=10 for each group). The PLRCA-CM results showed a marked increase in the key components' interaction/co-localization in RCMTs-mediated ACS, such as the NSUN1-BRD4 interaction. A semi-quantitative analysis demonstrated a significant difference in the PL-RCA-CM signals of NSUN1-BRD4 interaction between azacitidine-sensitive vs. azacitidine-resistant BM specimens. The data also showed a similar increase in NSUN1/2-eRNAPII interactions in clinical BM specimens. These results confirmed increased NSUN1/2-mediated ACS in clinical azacitidine-resistant MDS and AML BM specimens, compared to the azacitidine-sensitive MDS and AML BM specimens. The data demonstrated that the monocytic leukemia line SC is highly resistant to venetoclax, whereas other leukemia cell lines, such as Kasumi-1 (K1) and CCRF-CEM, were sensitive to venetoclax. The inventors then established the venetoclax-resistant leukemia cell line K1VR from the K-1 cells and demonstrated that knockdown of either NSUN1 or NSUN2 with the corresponding siRNAs could re-sensitize the venetoclax-resistant leukemia cell line K1VR to venetoclax, which demonstrated an important role of NSUN1 and NSUN2 in venetoclax resistance.

The inventors examined expression of key components of NSUN1/2-mediated ACS, along with other markers, in clinical BM specimens from normal controls and from the patients with cytogenetically normal low-grade MDS (L.G. MDS), high-grade MDS (H.G. MDS), refractory anemia with multilineage dysplasia (RCMD) subtype, and MDS with excess blasts (MDS-EB), respectively, and monocytic AML. Ten cases were selected for each group. The morphology and manual differential count of the BM cells were assessed because blasts in erythroid and monocytic AML cases are often negative for CD34, a useful marker for identifying hematopoietic stem cells and leukemic blasts. Immunohistochemical analysis demonstrated low expression levels of CD34, hnRNPK, NSUN1 and BRD4 in the normal bone marrow cells, and markedly increased expression levels of these proteins in the BM cells of the MDS and AML cases, and the increased expression of these proteins is correlated with the disease progression in these MDS/AML cases. Of note: There was a drastic difference in NSUN1 expression between normal BM cells and high-grade MDS/AML, which indicates a large therapeutic window for NSUN1-targeting therapy.

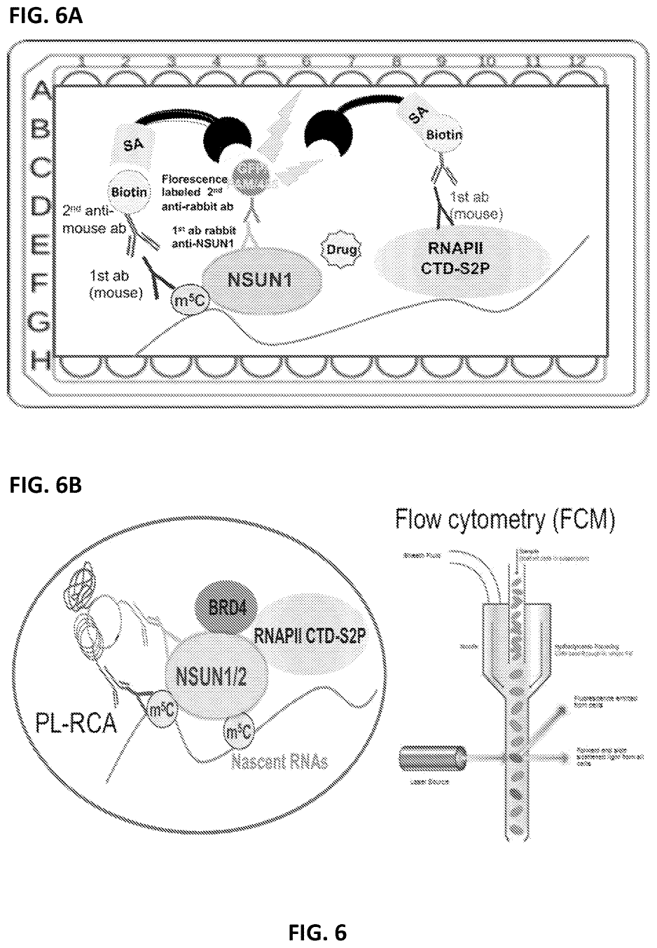

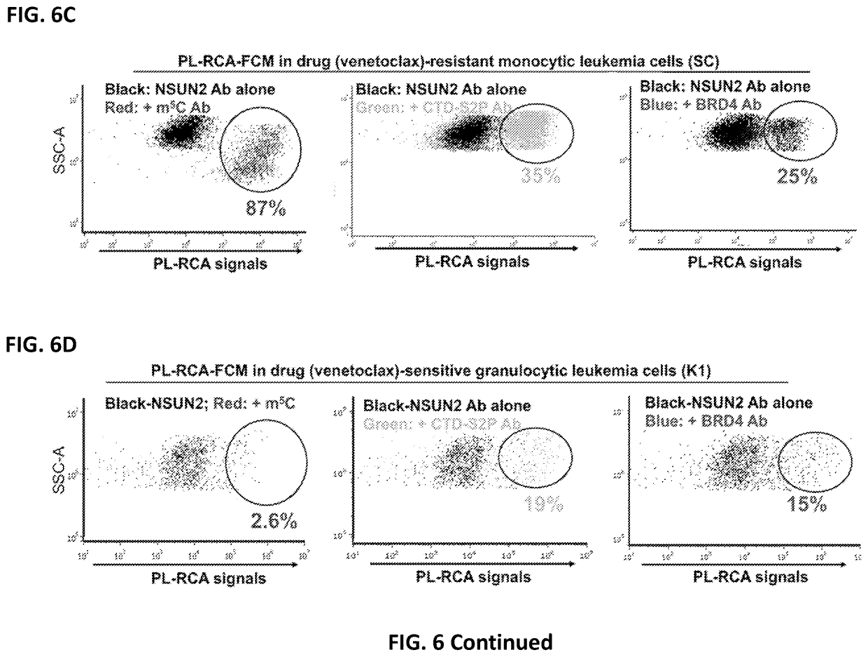

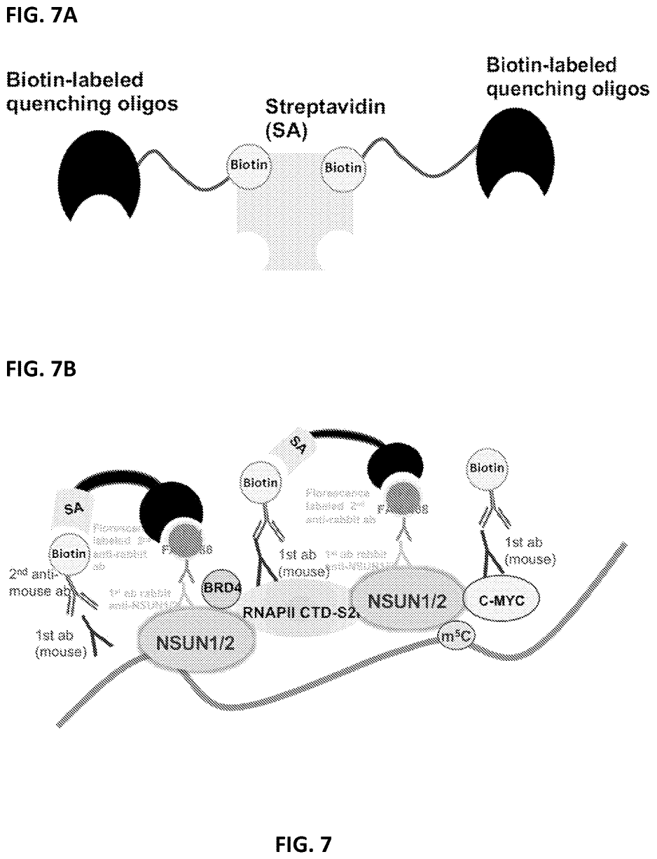

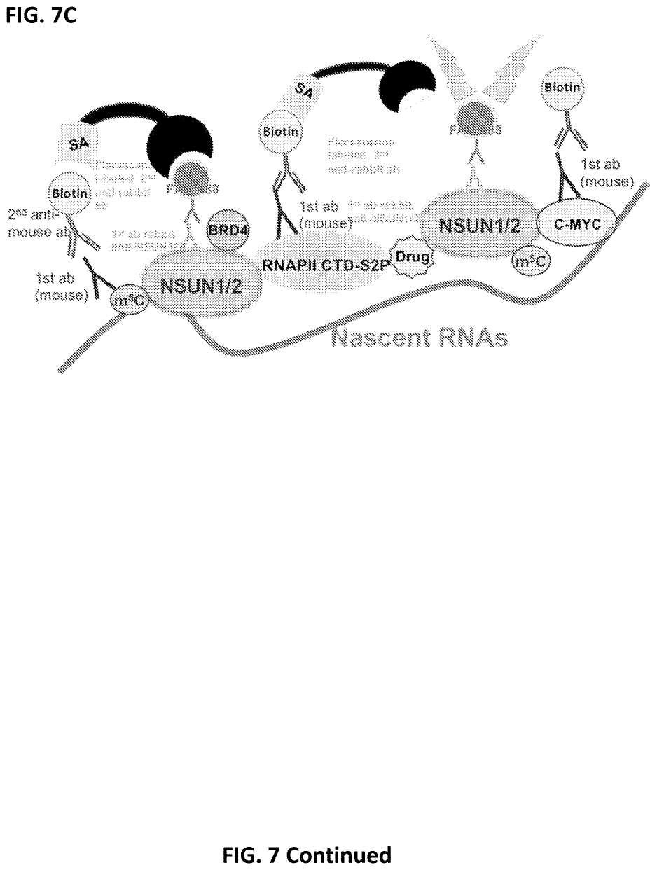

The PL-RCA-CM/imaging technique utilized in the inventors previous work (Cheng, J. X. et al., Nature Communications 2018) was suitable to visualize and semi-quantify the NSUN1/2-mediated ACS in cells. However, the previous technique had the disadvantage of having a low sensitivity and low throughput. To overcome this bottleneck, as described herein, the inventors developed NSUN1/2-targeting fluorescence quenching and release (FQR) plate assay (FIG. 6A) and PL-RCA flow cytometry (PL-RCA-FCM) technology (FIG. 6B) for screening of NSUN1/2-targeting drug libraries and quantitatively measuring NSUN1/2-mediated ACS with and without drugs in a lineage-specific and high-throughput manner. The new PL-RCA-FCM detected very strong PL-RCA signals (>2 logs) of the NSUN2-mediated interactions with m5C, RNAPII CTD2-S2P and BRD4, particularly in the venetoclax-resistant leukemia cells (SC) (FIG. 6C), in comparison to the venetoclax-sensitive K1 cells (FIG. 6D). These data demonstrated the importance of RCMTs in venetoclax resistance and provided a novel technology to analyze ACS in a lineage-specific and high-throughput manner.

The inventors then designed a protocol for functional screening of NSUN1/2-targeting supercomputing/AI-derived compound libraries as schematically presented in FIGS. 5A-5G. Briefly, the library compounds were obtained from various chemical companies/vendors and dissolved in DMSO, and then applied to 96-well plates that contained leukemia and/or other cancer cells that were resistant to venetoclax. Both the routine colorimetric assay (MTT assay) and the newly developed NSUN1/2-targeting fluorescence quenching and release (FQR) assay were performed to select bioactive compounds for further quantitative study to confirm that they targetNSUN1/2 and their associated ACS. As described below, the compound libraries screened through cancer cell proliferation and/or NSUN1/2 localization to the ACS successfully identified numerous promising compounds that can inhibit the leukemia cell growth and appear to target the NSUN1-mediated ACS (see Table 2). This success has proven the feasibility and efficacy of the described supercomputing/AI design and functional screening of the NSUN1/2-targeting drug libraries. In some aspects, screening is conducted using drug-resistant cell lines. In some aspects, screening is conducted using drug-sensitive cell lines. In some aspects, screening is conducted in leukemia cell lines. In some aspects, screening is conducted in other cancer cell lines besides leukemia (e.g., lung cancer cell lines). In some aspects, NSUN1/2-ACS inhibitors identified are verified in additional cell lines and/or animal models (see e.g., Tables 4-6).

Characterization and Optimization of NSUN1/2-Targeting Lead Compounds and their Structure-Activity Relationship (SAR) in Leukemia and Cancer Cells.

After completion of the initial screening of the NSUN1/2-targeting drug libraries in various drug-resistant leukemia and cancer cell lines, characterization of the targets (e.g., targeted NSUN1/2 binding modules) and action mechanisms for identified compounds is conducted. The SAR between NSUN1/2 and the putative small molecule NSUN1/2-ACS inhibitors can then be improved through compound derivatization. As described herein, supercomputing/AI re-docking the identified NSUN1-11 and NSUN1-17 compounds to the predicted NSUN1 protein structure revealed that both the compounds bind to the A-460 binding surface/module of NSUN1 protein. The inventors PL-RCA-FCM assays demonstrated that NSUN1-17 compound effectively disrupted the interactions between NSUN1 and eRNAPII and c-MYC, but that the compound had little effects on the interaction between NSUN1 and RNA:m5C or BRD4 (FIG. 5G). Based on these data, the inventors characterize the targets and SAR of identified compounds for lead compound NSUN1/2-ACS inhibition improvement.

The lead compounds are re-designed (e.g., derivatized) and a second-round of compound libraries is produced. The lead compounds were re-designed (e.g., derivatized) and a second-round of compound libraries was produced from positive hits described in Table 3.

Derivative compounds docking to the predicted NSUN1 and NSUN2 structures are re-simulated to characterize drug-target interactions. The second-round compound libraries are generated based on the best structural hits from the computational simulation. Functional screening(s) of the second-round libraries are performed, and validation of the targets and efficacies of identified lead compounds are determined. The derivatized lead compounds targeting various binding surfaces/modules of NSUN1 and NSUN2 proteins are chosen for further structural, molecular, functional, and in vivo animal studies.

As described above, thus far the crystal structures of NSUN1 and NSUN2 proteins have not yet been solved. The detailed structural information of RCMTs is only limited to NSUN6. However, NSUN1 and NSUN2 protein sequences share low homology with NSUN6 protein sequence, except the methyltransferase domain. Therefore, it would be extremely helpful to obtain the detailed structural information about NSUN1 and NSUN2 proteins for functional study and drug design.

Analysis of NSUN1 and NSUN2 crystal structures are performed. Briefly, protocols of high-throughput protein purification and quality assessment for crystallization and for x-ray crystallography, cryo-EM and NMR are followed to determine the SAR of NSUN1/2 proteins and the associated key factors with their targeting lead compounds.

Full length NSUN2 and truncated constructs were expressed and purified. These purified NSUN2s bound RNA substrates. Proteins and their complexes with small ligands will be crystallized. Diffraction data will be collected at sector 19 of the APS. Alternative beamtime is available at NSLS II and ALS beamlines. The inventors anticipate that the structures will be solved by molecular replacement using available models in the PDB or produced using AlfaFold2 modeling. The datasets will be processed, structures refined, validated and deposited to the PDB using standard crystallographic protocols. If deemed necessary, cryo-electron microscopy (cryo-EM) will be performed to determine structure and characterize complexes with the lead compound(s) binding to NSUN1 and NSUN2 protein structures as described by the literatures.

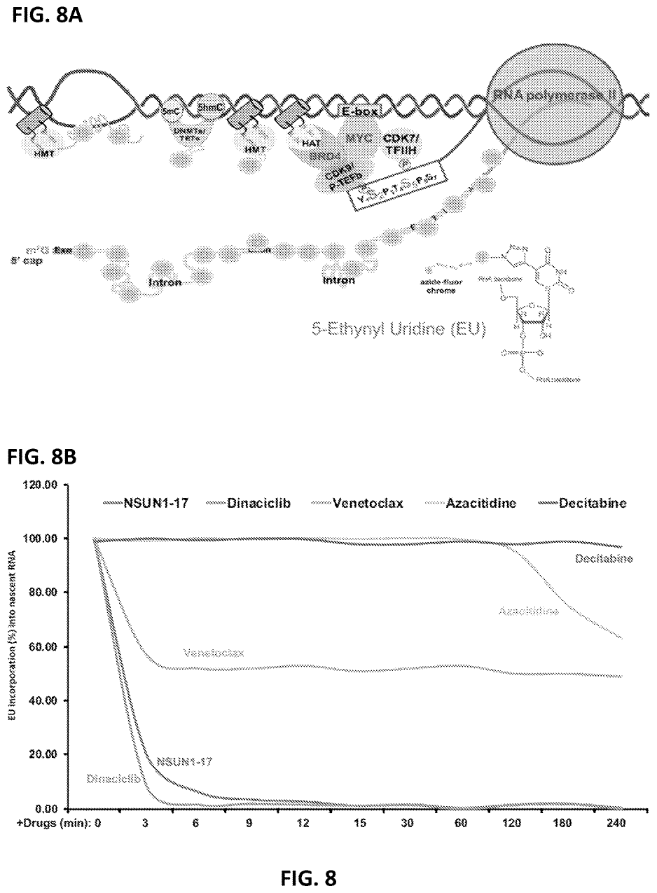

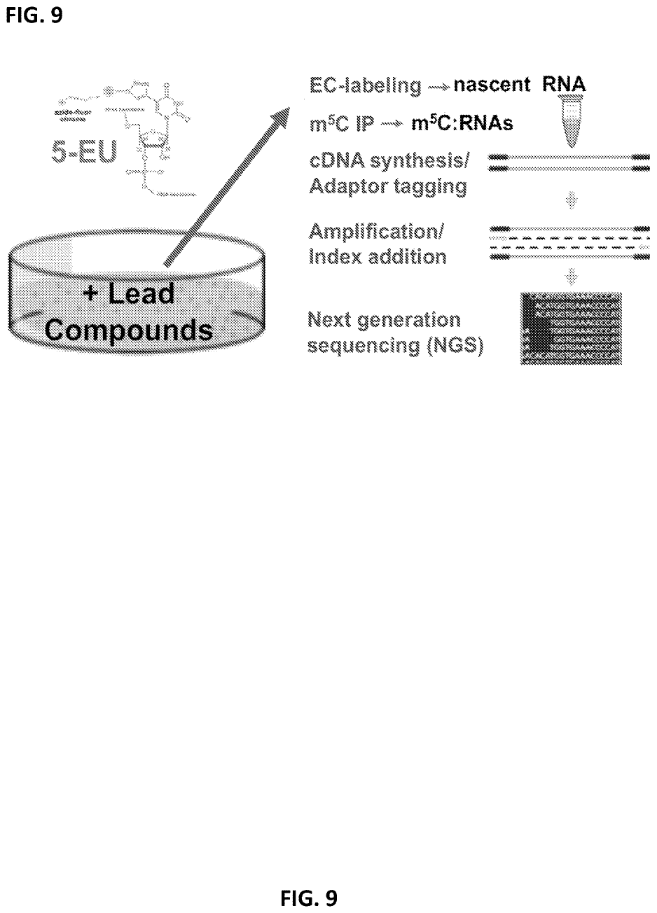

As described above, the importance of NSUN1/2-mediated ACS in cancer drug resistance has been demonstrated, and the development of novel NSUN1/2-targeting therapeutics is of great import. Importantly, the data provided herein in the form of EC-FCM assay results also demonstrates distinct drug-specific inhibition patterns of nascent RNA synthesis in leukemia cells that are responsive to the drug treatment (FIG. 8B). Based on these data, the inventors predict that the identified NSUN1/2-targeting lead compounds will have distinct and unique patterns/signatures of inhibition of nascent RNA synthesis and RNA:m5C, as well as disruptions of NSUN1/2-mediated ACS at the genome-wide level.

In some aspects, technologies provided herein, such as but not limited to, EC- and RNA:m5C immunoprecipitation (IP) coupled RNA sequencing (ECRNA-seq and RNA:m5C-IP-RNA-seq) technologies, can be utilized to characterize the patterns/signatures of nascent RNA synthesis and RNA:m5C inhibition induced by compounds in leukemia and cancer cell lines at the genome-wide level. In some aspects, both EC- and RNA:m5C-IP-RNA-seq, 2 million cells/per group will be treated with 1. No drug control (ctrl); 2. Lead compounds; 3. Venetoclax (Ven) and 4. Lead compounds(s) plus Ven, and then incubated with 5-ethynyl uridine (EU) to label (click) nascent RNAs. Total RNAs are isolated using TRIzol reagents and protocol (Invitrogen).

In some aspects, NSUN1/2-ACS inhibitors are validated and characterized in vivo using leukemia/tumor cell line xenograft (CDX) and/or patient-derived xenograft (PDX) mouse models.

In some aspects, the acute toxicity of compounds described herein are analyzed. Briefly, a single mouse is given a single injection (IP, IV, SC, IM or PO) of 400 mg/kg (or lower if the compound is anticipated to be extremely potent, e.g., natural products); a second mouse receives a dose of 200 mg/kg and a third mouse receives a single dose of 100 mg/kg. The mice are observed for a period of 2 weeks. They are sacrificed if they lose more than 20% of their body weight or if there are other signs of significant toxicity. If all 3 mice must be sacrificed, the next 3 dose levels (50, 35 and 12.5 mg/kg) are tested in a similar manner. This process is repeated until a tolerated dose is found. This dose is then designated the MTD and is used to calculate the amount of material administered to mice during antitumor testing. Dose volumes are generally 0.1 mL/10 grams body weight but may be up to 0.2 mL/10 grams of body weight for IP, IV, SC and PO routes. For the standard hollow fiber assay (HFA), the high and low dose levels are determined using the MTD as determined above using the formula below. High dose=[MTD×1.5]/4; Low dose=0.67×high dose. The standard vehicle used for both acute toxicity testing and HFA is 10% DMSO in saline/0.05% Tween 80.

In some aspects, NSUN1/2-ACS inhibitors are validated as compounds suitable for overcoming drug resistance in tumor models (e.g., mouse models).

In some aspects, animal models are necessary for assessing the efficacy of potential drugs or lead compounds to determine which compounds are likely to move to clinical trials at the next step of drug development. Since cell lines (leukemia/cancer cell lines) derived xenograft (CDX) and patient-derived xenograft (PDX) AML mouse models have been extensively used to study the efficacies of venetoclax and its combination therapies, the inventors will adopt published protocols to assess the efficacies of optimized compounds alone, and in combination with venetoclax in immunocompetent syngeneic CDX mice and immunodeficient PDX mice.

In some aspects, associated experimental plan/procedures comprise: 1) For syngeneic CDX experiments, mice with the same genetic background as the mouse leukemia cell lines will be used. More specifically, in certain aspects, C1498 cell lines, a mouse AML cell line with moderate to high resistance to venetoclax, and immunocompetent syngeneic C57BL/6J (Jackson Laboratory) will be used for mouse leukemia cell transplant. In some aspects, immunodeficient NOD/SCID (NS) mice (Jackson Laboratory) will be used for human leukemia and cancer CDX and PDX models. More specifically, in some aspects, human monocytic leukemia cell line SC and human lung cancer cell line NCI-H82, both of which are resistant to venetoclax, will be used to generate CDX mouse models. CDX and PDX AML models have been extensively used to test venetoclax-based combination therapies. Briefly, the aforementioned venetoclax-resistant leukemia and solid tumor cell lines (e.g., 0.5×10⁶ cells/mouse) will be intravenous (IV) injection via the tail veins and subcutaneous injection (SQ) over the shoulders, respectively. Sex-matched and 8-12-week-old mice (n=6/group) will be used for each identified lead compound, and the experiment groups will include 1. No drug control (ctrl), 2. Lead compound alone, 3. Venetoclax alone, and 4. Lead compound plus venetoclax. Of note: Patients with venetoclax-resistant AML usually have a high leukemic blast counts in peripheral blood (PB) and bone marrow (BM), and thus sufficient leukemic blasts/cells from those patients for PDX models should be readily available. Due to the concern of the heterogeneity nature of clinical specimens, in some aspects, the inventors will collect the leukemia blasts/cells from both sexes and using the age- and sex-matched specimens for comparison. In some aspects, the leukemia cells will be allowed to engraft for 6 days, and then the mice will be randomly assigned to oral gavage with vehicle control, the lead compound(s) (e.g., 25 mg/kg), venetoclax (e.g., 25 mg/kg), and the combination of the lead compound(s) and venetoclax, by oral gavage, for 5 days per week for 4 weeks. For all mice that received tumor cell transplantations, blood will be drawn once per week to monitor the development of leukemia, and the mice will be closely monitored for signs of solid tumor formation and leukemia progression, such as weight loss, lethargy, and hind limb paralysis. The densitometry measurement of treated animals and survival graphs will be conducted, and the mice will be euthanized by using carbon dioxide (C₀₂) euthanasia chamber. Statistical comparison analysis of survival rates and blast counts in PB and BM (for AML CDX and PDX) as well as tumor size and metastasis (for tumor CDX cell lines) in those groups of mice to assess the efficacies of lead compounds.

In some aspects, NSUN1/2-ACS inhibitors are elucidated in vivo targets and action mechanisms of lead compounds are determined.

In some aspects, NSUN1/2-ACS inhibitors may have on both the host healthy cells and host cancer cells. mouse and human leukemia and cancer cells and the host organs/tissues as well as tumor-host interactions and microenvironment. In some aspects, the inventors will focus on peripheral blood (PB), bone marrow (BM) and local tumor/cancer mass lesions as well as the major organs, including lungs, livers and kidneys. In some aspects, the inventors will specifically study the compounds/drugs-induced histological, immunophenotypic and molecular changes in both tumor/leukemia cells and host cells by morphological evaluation, immunohistochemical and flow cytometric analysis as well as molecular and protein technologies that can dissect NSUN1/2-mediated ACS and identify drug/compound-specific RNA epigenetics signatures. As described in the previous sections and the inventors previous publications, dot blot assay and high sensitivity mass spectrometry will be used for analysis of RNA:m5C; EC- and PL-RCA-coupled stimulated emission depletion (STED) confocal microscopy (CM) will be used to study drug/compound-induced changes in NSUN1/2-mediated ACS, while EC-/PL-RCAFCM will be used to quantitatively analyze those changes in a high-throughput and lineage-specific manner. In some aspects, the inventors perform for bulk and single-cell RNA sequencing by using 10× Genomics kits and following the company's protocols. In certain aspects, the inventors perform UV crosslinking and immunoprecipitation-based RNA sequencing, i.e., CLIP- and RNA:m5C-IP-RNA-seq for analysis of genome-wide drug/compound-induced changes in NSUN1/2-mediated ACS and identification of drug/compound-specific RNA epigenetic signature.

In certain aspects, the tissues of tumors and major organs from experiments described herein will be processed by following published protocols. Briefly, bone marrow cores will be decalcified following the conventional protocol of EDTA at 25 C for the best quality of tissue preservation. Tissues will be put in 4% paraformaldehyde for 10 hours at 4° C. and then paraffin-embedded for sectioning and hematoxylin and eosin (H&E) staining. Immunohistochemistry (IHC) and flow cytometry (FCM) with antibodies against NSUN1, NSUN2 and other ACS key components and lineage markers will be performed on the mouse tissues according to published protocols. The H&E and IHC sections and FCM data will be reviewed and analyzed. Statistical analysis: the significance between the rankings of two variables was determined by Spearman's rank correlation co-efficient and the Mantel-Cox test used to assess significance between Kaplan-Meier survival curves, both using GraphPad Prism (GraphPad Software).

In certain aspects, commercially available cell lines are utilized herein. Ten leukemia cell lines with different lineages/differentiations were obtained from the American Tissue Culture Collection (ATCC) and the Deutsche Sammlung von Mikroorganismen und Zellkulturen company (DSMZ), which include OCI-M2, K562, MonoMac6, SC, THP1, U937, MV4-11, HL-60, KASUMI-1 and NB4. Which include OCI-M2 was purchased from the DSMZ company, and the rest leukemia cell lines were purchased from ATCC. In some aspects, four previously tested leukemia cell lines, including the erythroid leukemia cell lines, OCI-M2 and K562; the monocytic leukemia cell line, SC and the granulocytic leukemia cell line (HL-60) are utilized. In addition, in some aspects, two B-cell lines from ATCC, namely, Toledo (diffuse large B-cell lymphoma) and CCRF-SB (acute B-lymphoblastic leukemia, B-ALL), and two T-cell lines from ATCC, specifically, HH (cutaneous T-cell lymphoma) and CCRFCEM (acute T-lymphoblastic leukemia, T-ALL) are utilized.

In certain aspects, cell lines developed in the inventors laboratory are utilized herein. The inventors have developed multiple drug resistant cell lines including 5-AZA-resistant leukemia cells, including M2AR and SCAR, that developed from the original 5-AZA-sensitive OCIM2 and SC, respectively, as well as venetoclax-resistant cell lines K1VR and M2VR, which were developed from the original Venetoclax-sensitive Kasumi-1 and OCI-M2 cell lines.

In certain aspects, antibodies utilized herein are commercially available and were tested and verified in our previous study. The key antibodies include: NSUN1 and NSUN2 antibodies from the Santa Cruz biotechnology; DNMT2 and NSUN3 from the Signa-Aldrich company, and RNAPII CTD-S2P and SRB4 antibodies from the CellSignaling company. In certain aspects, FAC-sorting antibodies are purchased from the BD Biosciences company.

In certain aspects, Azacitidine (5-azacytidine, 5-AZA, Vidaza), Decitabine (5-aza-2′-deoxycytidine, Dacogen), and cytarabine (cytosine arabinoside, ARA-C) were and are purchased from Sigma-Aldrich. Venetoclax and Dinaciclib (SCH727965) were and are purchased from the SelleckChem company.

In certain aspects, a small molecule NSUN1/2-ACS inhibitor binds to the A-460 module of NSUN1 (SEQ ID NO: 1). In certain aspects, an A-460 module of NSUN1 comprises interactions of amino acids: L-aa 459; D-aa 460; A-aa 461; P-461; C-aa 463; S-aa 464; Q-aa 491; Y-aa 514; C-aa 515; T-aa 516; and/or C-aa 517, of NSUN1 (SEQ ID NO: 1).

In certain aspects, a small molecule NSUN1/2-ACS inhibitor binds to the A-691 module of NSUN2 (SEQ ID NO: 2).

In certain aspects, a small molecule NSUN1/2-ACS inhibitor binds to the A-460 module of NSUN1 and the A-691 module of NSUN2.

In certain aspects, a small molecule NSUN1/2-ACS inhibitor binds to at least the A-460 module of NSUN1 and/or the A-691 module of NSUN2, and at least one additional NSUN1/2 module.

In certain aspects, compound NSUN1-17-3 and/or derivatives thereof bind to both the A-460 module of NSUN1 and the A-691 module of NSUN2.

In certain aspects, compound NSUN1-17 and/or derivatives thereof bind to both the A-460 module of NSUN1 and the A-691 module of NSUN2. In certain aspects, compound NSUN1-17 and/or derivatives thereof binds to A-316 and A-460 of NSUN1, and A-279, A-580, A-271, and A-162 of NSUN2.

In certain aspects, compound NSUN1-73 and/or derivatives thereof binds to A-316, A-460, and A-371 of NSUN1, and A-89, A-580, and A-271 of NSUN2.

In certain aspects, compound NSUN1-74 and/or derivatives thereof bind to both the A-460 module of NSUN1 and the A-691 module of NSUN2.

In certain aspects, compound NSUN1-64 and/or derivatives thereof bind to both the A-460 module of NSUN1 and the A-691 module of NSUN2.

In certain aspects, compound NSUN1-11 and/or derivatives thereof bind to both the A-460 module of NSUN1 and the A-691 module of NSUN2.

In certain aspects, compound NSUN2-11 and/or derivatives thereof bind to the A-460 module of NSUN1, and the A-691, A-271, and A-162 module of NSUN2.

In certain aspects, compound NSUN2-19 and/or derivatives thereof bind to A-316 of NSUN1, and A-279 and A-691 of NSUN2.

In certain aspects, a screen for identifying small molecule NSUN1/2-ACS inhibitors comprises a cell proliferation inhibition assay.

In certain aspects, a screen for identifying small molecule NSUN1/2-ACS inhibitors comprises a FQR plate assay and/or a PL-RCA flow cytometry (PL-RCA-FCM) assay.

In certain aspects, a small molecule NSUN1/2-ACS inhibitor has substantially no effect (e.g., inhibitory) on NSUN1/2-BRD4 interactions.

In certain aspects, a small molecule NSUN1/2-ACS inhibitor has substantially no effect (e.g., inhibitory) on NSUN1/2-m5C interactions.

In certain aspects, a small molecule NSUN1/2-ACS inhibitor has significant (e.g., inhibitory) effects on NSUN1/2-RNAPII interactions.

In certain aspects, a small molecule NSUN1/2-ACS inhibitor has significant effects (e.g., inhibitory) on NSUN1/2-RNA Polymerase Mitochondrial (POLRMT) interactions.

In certain aspects, a small molecule NSUN1/2-ACS inhibitor has significant effects (e.g., inhibitory) on NSUN1/2-mitochondrial translation initiation factor 3 (MTIF3) intreactions.

In certain aspects, a small molecule NSUN1/2-ACS inhibitor has significant effects (e.g., inhibitory) on NSUN1/2-Ribosome complex interactions.

In certain aspects, a small molecule NSUN1/2-ACS inhibitor has significant effects (e.g., inhibitory) on NSUN1/2-c-MYC interactions.

In certain aspects, a small molecule NSUN1/2-ACS inhibitor has substantially no effect (e.g., inhibitory) on NSUN1/2-BRD4 and/or NSUN1/2-m5C interactions, and has significant effects (e.g., inhibitory) on NSUN1/2-RNAPII, NSUN1/2-POLMRT, NSUN1/2-MTIF3, and/or NSUN1/2-c-MYC interactions.

In some aspects, a small molecule NSUN1/2-ACS inhibitor has significant effects (e.g., inhibitory) on NSUN1/2-BRD4, NSUN1/2-m5C, NSUN1/2-RNAPII, NSUN1/2-POLMRT, NSUN1/2-MTIF3, and/or NSUN1/2-c-MYC interactions.

In certain aspects, a small molecule NSUN1/2-ACS inhibitor provides at least, or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, in vitro and/or in vivo cell growth inhibition of NSUN1/2-ACS dependent tumor cells.

In certain aspects, a small molecule NSUN1/2-ACS inhibitor provides at least, or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, inhibition of NSUN1/2 binding with eRNAPII and/or c-MYC.

In certain aspects, provided herein are methods of treatment comprising administration of NSUN1-17 and/or derivatives thereof. In certain aspects, provided herein are methods of treatment comprising administration of NSUN2-11 and/or derivatives thereof. In certain aspects, provided herein are methods of treatment comprising administration of NSUN1-17 and/or derivatives thereof, and NSUN2-11 and/or derivatives thereof. In certain aspects, provided herein are methods of treatment comprising administration of NSUN1-17 and/or derivatives thereof, and 5-AZA. In certain aspects, provided herein are methods of treatment comprising administration of NSUN1-17 and/or derivatives thereof, and venetoclax. In certain aspects, provided herein are methods of treatment comprising administration of NSUN2-11 and/or derivatives thereof, and 5-AZA. In certain aspects, provided herein are methods of treatment comprising administration of NSUN2-11 and/or derivatives thereof, and venetoclax.

In certain aspects, a small molecule NSUN1/2-ACS inhibitor provides at least, or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, inhibition of NSUN1/2 binding with RNA Polymerase Mitochondrial (POLRMT).

In certain aspects, a small molecule NSUN1/2-ACS inhibitor provides at least, or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, inhibition of NSUN1/2 binding with translational machinery (e.g., ribosomal complexes).

In certain aspects, methods and compositions comprising small molecule NSUN1/2-ACS inhibitors include a concentration of compound at exactly, at least, no more than, or about 1 M, 0.9 M, 0.8 M, 0.7 M, 0.6 M, 0.5 M, 0.4 M, 0.3 M, 0.2 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M, or any range derivable therein. In some aspects, methods and compositions comprising small molecule NSUN1/2-ACS inhibitors include a concentration of compound at exactly, at least, no more than, or about 0.001 M to 1,000 μM, 0.01 M to 100 M, 0.1 μM to 100 M, 1 M to 100 M, or 1 M to 10 μM, or any range derivable therein.

In some aspects, provided herein are compositions and methods comprising use of bioactive hits from screening of newly generated NSUN1 and NSUN2 targeting small molecule libraries that were validated ex vivo in leukemia and lung cancer cell lines. In some aspects, provided herein are bioactive positive hits that inhibit tumor cell growth in a non-lineage specific manner, e.g., inhibit both leukemia and lung cancer cell lines.

In some aspects, a bioactive positive hit that inhibits tumor cell growth in a non-lineage specific manner comprises, consists essentially of, or consists of NSUN1-11, NSUN1-1, NSUN1-67, NSUN1-74, NSUN1-30, NSUN1-18, NSUN1-17.16, NSUN1-17.17, NSUN1-17.22, NSUN1-17.20, NSUN1-17.3, and/or NSUN1-17.21. In some aspects, a bioactive compound that inhibits tumor cell growth in a non-lineage specific manner comprises, consists essentially of, or consists of a derivative of NSUN1-11, NSUN1-1, NSUN1-67, NSUN1-74, NSUN1-30, NSUN1-18, NSUN1-17.16, NSUN1-17.17, NSUN1-17.22, NSUN1-17.20, NSUN1-17.3, and/or NSUN1-17.21.

In some aspects, a bioactive positive hit that inhibits tumor cell growth in a non-lineage specific manner comprises, consists essentially of, or consists of NSUN2-11, NSUN2-37, NSUN2-4, NSUN2-24, NSUN2-10, NSUN2-67, NSUN2-5, NSUN2-14, NSUN2-13, NSUN2-73, NSUN2-88, NSUN2-64, and/or NSUN2-85. In some aspects, a bioactive compound that inhibits tumor cell growth in a non-lineage specific manner comprises, consists essentially of, or consists of a derivative of NSUN2-11, NSUN2-37, NSUN2-4, NSUN2-24, NSUN2-10, NSUN2-67, NSUN2-5, NSUN2-14, NSUN2-13, NSUN2-73, NSUN2-88, NSUN2-64, and/or NSUN2-85.

In some aspects, provided herein are bioactive positive hits that inhibit tumor cell growth in a lineage preferential manner, e.g., preferentially inhibiting leukemia cells over lung cancer cells, or vice versa. In some aspects, provided herein are bioactive positive hits that inhibit tumor cell growth specific manner.

In some aspects, a bioactive positive hit that inhibits tumor cell growth in a leukemia preferential manner comprises, consists essentially of, or consists of NSUN1-24, NSUN1-27, NSUN1-31, NSUN1-64, NSUN1-25, NSUN1-68, NSUN1-9, NSUN1-54, NSUN1-23, and/or NSUN1-10.

In some aspects, a bioactive positive hit that inhibits tumor cell growth in a leukemia preferential manner comprises, consists essentially of, or consists of NSUN2-20, NSUN2-21, NSUN2-19, NSUN2-39, NSUN2-38, NSUN2-98, NSUN2-62, and/or NSUN2-18.

In some aspects, a bioactive positive hit that inhibits tumor cell growth in a lung cancer preferential manner comprises, consists essentially of, or consists of NSUN1-19, NSUN1-22, NSUN1-56, and/or NSUN1-2.

In some aspects, a bioactive positive hit that inhibits tumor cell growth in a lung cancer preferential manner comprises, consists essentially of, or consists of NSUN2-35, NSUN2-13, NSUN2-55, NSUN2-30, NSUN2-53, and/or NSUN2-32.

Also disclosed herein, in some aspects, is identification of bioactive negative hits that promote tumor cell growth in a lineage or non-lineage dependent manner. In some aspects, bioactive negative hits are informative in identifying NSUN1 and/or NSUN2 binding modules appropriate for targeting and inhibition.

In some aspects, a bioactive negative hit for NSUN1 and/or NSUN2 mediated tumor cell growth inhibition comprises, consists essentially of, or consists of NSUN1-38, NSUN1-33, NSUN1-36, NSUN1-28, NSUN1-4, NSUN1-32, NSUN1-43, NSUN1-44, NSUN1-46, NSUN1-41, NSUN2-43, NSUN2-51, NSUN2-41, NSUN2-29, NSUN2-23, NSUN2-55, NSUN2-22, NSUN2-26, NSUN2-2, NSUN2-52, NSUN2-49, NSUN2-56, NSUN2-66, NSUN2-40, and/or NSUN2-95.

In some aspects, methods and/or compositions described herein do not comprise NSUN1 and/or NSUN2 binding small molecules NSUN1-38, NSUN1-33, NSUN1-36, NSUN1-28, NSUN1-4, NSUN1-32, NSUN1-43, NSUN1-44, NSUN1-46, NSUN1-41, NSUN2-43, NSUN2-51, NSUN2-41, NSUN2-29, NSUN2-23, NSUN2-55, NSUN2-22, NSUN2-26, NSUN2-2, NSUN2-52, NSUN2-49, NSUN2-56, NSUN2-66, NSUN2-40, and/or NSUN2-95.

In certain aspects, NSUN1-11 and/or a derivative thereof interacts (contact unless otherwise noted) with NSUN1 A-460 binding module at amino acid residues ALA394 (acceptor), ALA461, ARG 421 (donor), ASP 460, CYS517, GLN369, GLY370, LEU368, LYS398, LYS471 (donor), PR0395, PR0462, SER737 (donor), THR311, VAL310, and/or VAL468. In certain aspects NSUN1-11 and/or a derivative thereof interacts (contact unless otherwise noted) with NSUN1 A-219 binding module at amino acid residues ALA255 (acceptor), ARG257, ASP245, GLN256, GLU216, GLU217, GLU258, LYS241 (donor), PHE219, and/or PRO218. In certain aspects NSUN1-11 and/or a derivative thereof interacts (contact unless otherwise noted) with NSUN1 A-371 binding module at amino acid residues ASP460, CYS517, GLN369, GLY370, GLY397, LEU368, LYS398 (donor), LYS471, PR0309, PR0395, SER373 (donor), THR311 (acceptor), and/or VAL310. In certain aspects NSUN1-11 and/or a derivative thereof interacts (contact unless otherwise noted) with NSUN1 A-316 binding module at amino acid residues GLN404, LEU317, LEU405, LYS343, LYS407, MET378, THR316, THR344 (donor), TRP341 (acceptor), and/or TYR401. In certain aspects NSUN1-11 and/or a derivative thereof interacts (contact unless otherwise noted) with NSUN2 A-202 binding module at amino acid residues HIS199 (donor), LYS84, MET202 (acceptor), PR0205, PR0674, SER85 (donor), THR610, TYR609, and/or TYR83.

In certain aspects, NSUN1-17 and/or a derivative thereof interacts (contact unless otherwise noted) with NSUN1 A-460 binding module at amino acid residues ALA417, ALA461, ARG421, ASN418, ASP416, ASP460 (acceptor), ASP577, CYS392 (acceptor), CYS517, LYS471 (donor), PHE579, PR0462, SER372, SER373, and/or VAL468. In certain aspects NSUN1-17 and/or a derivative thereof interacts (contact unless otherwise noted) with NSUN1 A-219 binding module at amino acid residues ALA255, ARG257, ASP245 (acceptor), GLN244, GLN256 (donor), GLU216 (acceptor), GLU258, GLY248, ILE249, PHE219, and/or PRO218. In certain aspects NSUN1-17 and/or a derivative thereof interacts (contact unless otherwise noted) with NSUN1 A-371 binding module at amino acid residues ARG308 (acceptor), ASP460 (acceptor), GLN369, GLY370, GLY397, LEU368, LYS398, LYS417 (donor), PR0395, SER373, SER470, and/or VAL310. In certain aspects NSUN1-17 and/or a derivative thereof interacts (contact unless otherwise noted) with NSUN1 A-316 binding module at amino acid residues ALA381, GLN383 (donor), GLN404, GLU374, GLY543 (acceptor), LEU405 (acceptor), LYS407, MET378, PRO382, and/or THR316. In certain aspects NSUN1-17 and/or a derivative thereof interacts (contact unless otherwise noted) with NSUN2 A-89 binding module at amino acid residues CYS673, GLN672, GLU89 (acceptor), LYS88, MET202, PR0674, SER85, and/or TYR609. In certain aspects NSUN1-17 and/or a derivative thereof interacts (contact unless otherwise noted) with NSUN2 A-162 binding module at amino acid residues ARG160, ARG278, ASN280, ASN418 (acceptor), GLN161, GLU162 (acceptor), LYS279, SER189, and/or THR276. In certain aspects NSUN1-17 and/or a derivative thereof interacts (contact unless otherwise noted) with NSUN2 A-301 binding module at amino acid residues ARG301, ARG389 (donor), CYS502, GLU308 (acceptor), GLY500, LEU300, LYS338, PR0390, SER244, THR304 (acceptor), THR391, and/or VAL501.

In certain aspects, NSUN2-11 and/or a derivative thereof interacts (contact unless otherwise noted) with NSUN2 A-89 binding module at amino acid residues ALA670, ASP669, GLN672, GLU89 (acceptor), GLY703, HIS92, LEU671, LYS88, MET701, MET702, PR0668, and/or SER85. In certain aspects, NSUN2-11 and/or a derivative thereof interacts (contact unless otherwise noted) with NSUN2 A-162 binding module at amino acid residues ARG160, ARG278, ASN280, ASN418 (acceptor), GLN161, GLU162 (acceptor), ILE281, LYS279, THR276 (donor), and/or THR419. In certain aspects, NSUN2-11 and/or a derivative thereof interacts (contact unless otherwise noted) with NSUN2 A-202 binding module at amino acid residues ASN203, HIS199, LYS84, MET202 (acceptor), PR0205, SER85 (acceptor), THR610, TYR609 (acceptor), TYR83, and/or VAL204. In certain aspects, NSUN2-11 and/or a derivative thereof interacts (contact unless otherwise noted) with NSUN2 A-279 binding module at amino acid residues ALA76, ARG133, ARG160, ASP268, CYS321, GLN161, GLU162 (acceptor), LEU136, LYS279, SER165, and/or THR77 (acceptor). In certain aspects, NSUN2-11 and/or a derivative thereof interacts (contact unless otherwise noted) with NSUN2 A-301 binding module at amino acid residues ARG301, ARG305, ARG389, CYS502, GLU308 (acceptor), LYS338, PR0390, PR0504, SER244, SER245, THR304 (donor), and/or THR391. In certain aspects, NSUN2-11 and/or a derivative thereof interacts (contact unless otherwise noted) with NSUN2 A-580 binding module at amino acid residues ARG551, CYS758, GLU517, LEU223, LYS219, LYS516 (donor), PR0760, and/or TYR222. In certain aspects, NSUN2-11 and/or a derivative thereof interacts (contact unless otherwise noted) with NSUN2 A-691 binding module at amino acid residues ARG230, ASN157 (acceptor), GLU154, GLU694, GLY156, GLY82, HIS226, LEU231, LYS229 (acceptor), SER155 (acceptor), SER232 (acceptor), THR81, and/or TYR83. In certain aspects, NSUN2-11 and/or a derivative thereof interacts (contact unless otherwise noted) with NSUN2 A-700 binding module at amino acid residues ARG700 (donor), GLN147, GLU705, GLY708, LEU696, LEU707, LYS144, SER151 (donor), TYR98, VAL150, VAL706 (acceptor). In certain aspects, NSUN2-11 and/or a derivative thereof interacts (contact unless otherwise noted) with NSUN1 A-219 binding module at amino acid residues ARG257, ASP215, ASP245 (acceptor), GLN256, GLU216, GLU217, GLU258, GLU259, ILE249, LYS241, PHE219 (acceptor), and/or PR0218. In certain aspects, NSUN2-11 and/or a derivative thereof interacts (contact unless otherwise noted) with NSUN1 A-316 binding module at amino acid residues ARG320, GLY543, LEU405, LYS343 (donor), MET378, SER342, THR316, THR319, THR344 (donor), TRP341 (acceptor), and/or TYR401. In certain aspects, NSUN2-11 and/or a derivative thereof interacts (contact unless otherwise noted) with NSUN1 A-371 binding module at amino acid residues ASP460 (acceptor), CYS517, GLN269, GLY397, LYS398, LYS471, PR0395, SER373, and/or VAL310. In certain aspects, NSUN2-11 and/or a derivative thereof interacts (contact unless otherwise noted) with NSUN1 A-460 binding module at amino acid residues ALA461, ARG421, ASP460 (acceptor), ASP577, CYS392, CYS517, GLN369, GLY370, GLY397, LEU368, LYS398, LYS471, MET367, PHE579, PR0395, PR0462, SER372 (donor), SER373 (acceptor), and/or VAL468.

In certain aspects, NSUN2-37 and/or a derivative thereof interacts (contact unless otherwise noted) with NSUN2 A-89 binding module at amino acid residues ALA670, CYS673, GLN672, GLU89 (acceptor), HIS92, LYS84, LYS88 (donor), MET202, and/or SER85. In certain aspects, NSUN2-37 and/or a derivative thereof interacts (contact unless otherwise noted) with NSUN2 A-162 binding module at amino acid residues ARG160, ARG278, ASN418, GLN161, GLU162 (acceptor), GLY420, ILE281, LYS279, PR0390, THR276 (acceptor), and/or THR419. In certain aspects, NSUN2-37 and/or a derivative thereof interacts (contact unless otherwise noted) with NSUN2 A-202 binding module at amino acid residues CYS673, GLN672, GLU662, LYS660 (donor), MET202, PR0205, PR0613, PR0663, PR0674, SER85, TYR609 (acceptor), and/or TYR612. In certain aspects, NSUN2-37 and/or a derivative thereof interacts (contact unless otherwise noted) with NSUN2 A-301 binding module at amino acid residues ARG389, CYS502, GLU337 (acceptor), GLY503, HIS385, PHE393, PR0390, PR0394, PR0395, PR0504, PR0505, THR386, and/or THR391. In certain aspects, NSUN2-37 and/or a derivative thereof interacts (contact unless otherwise noted) with NSUN1 A-371 binding module at amino acid residues ALA394, ASP460 (acceptor), CYS517, LYS398, LYS471 (donor), PR0307, PR0395, SER373, and/or SER470. In certain aspects, NSUN2-37 and/or a derivative thereof interacts (contact unless otherwise noted) with NSUN1 A-316 binding module at amino acid residues GLN404, GLY543, LEU317, LEU405, LYS343, LYS407, MET378, THR316, THR344 (donor), and/or TYR401 (donor). In certain aspects, NSUN2-37 and/or a derivative thereof interacts (contact unless otherwise noted) with NSUN1 A-460 binding module at amino acid residues ALA394, ALA417, ALA461, ARG421, ASN418, ASP416, ASP460 (acceptor), CYS392, CYS517, GLN369, LEU368 (acceptor), LYS398, MET367, PR0395, PR0462, and/or SER373.

In certain aspects, NSUN2-35 and/or a derivative thereof interacts (contact unless otherwise noted) with NSUN2 A-89 binding module at amino acid residues ALA670, ARG700 (acceptor), ASP669, CYS93, GLN672, GLU89 (acceptor), GLY703, HIS92, LEU671, LYS88, LYS97 (donor), MET701, and/or PR0668. In certain aspects, NSUN2-35 and/or a derivative thereof interacts (contact unless otherwise noted) with NSUN2 A-162 binding module at amino acid residues ARG160, ARG278 (acceptor), ASN280, ASN418, GLU162 (acceptor), LYS279, TH1R276, and/or THR419. In certain aspects, NSUN2-35 and/or a derivative thereof interacts (contact unless otherwise noted) with NSUN2 A-202 binding module at amino acid residues GLN672, HIS199 (donor), LYS84, MET202 (acceptor), PR0205, PR0674, SER85, TYR609, TYR83, and/or VAL204. In certain aspects, NSUN2-35 and/or a derivative thereof interacts (contact unless otherwise noted) with NSUN2 A-301 binding module at amino acid residues ARG301, ARG305, ARG389, CYS502, GLU308 (acceptor), HIS385, LEU300, LYS338, PR0390, SER244, THR304, and/or THR391. In certain aspects, NSUN2-35 and/or a derivative thereof interacts (contact unless otherwise noted) with NSUN2 A-580 binding module at amino acid residues ASN218, ASN240 (acceptor), ASP518, GLU517, GLU576, ILE581, LEU571, LYS516 (donor), LYS579, MET578, PHE515, PHE520, PR0519, SER575, and/or VAL580 (acceptor). In certain aspects, NSUN2-35 and/or a derivative thereof interacts (contact unless otherwise noted) with NSUN2 A-691 binding module at amino acid residues ALA200, ARG230, ASN157 (acceptor), GLN193, GLU123, GLU196, GLU694, GLY82, HIS199, HIS697, LEU125, LEU170, LEU231, MET197, SER155, SER232, TYR121, and/or TYR83. In certain aspects, NSUN2-35 and/or a derivative thereof interacts (contact unless otherwise noted) with NSUN1 A-219 binding module at amino acid residues ARG238 (donor), ARG251, ASP245 (acceptor), GLN244, GLU217, GLY248, LYS241 (donor), PHE219, and/or VAL220. In certain aspects, NSUN2-35 and/or a derivative thereof interacts (contact unless otherwise noted) with NSUN1 A-371 binding module at amino acid residues ALA371, ARG308 (acceptor), ASN575 (acceptor), ASP336, GLY370, HIS574, LEU338, LYS471 (donor), MET576, PR0309, PR0337, THR311, TYR349, VAL310, and/or VAL347. In certain aspects, NSUN2-35 and/or a derivative thereof interacts (contact unless otherwise noted) with NSUN1 A-460 binding module at amino acid residues ALA394, ARG421, ASP416, ASP460 (acceptor), CYS392, CYS517, LEU368 (acceptor), LYS398, MET367, PR0395, PR0462, SER373, and/or TYR361 (donor). In certain aspects, NSUN2-35 and/or a derivative thereof interacts (contact unless otherwise noted) with NSUN1 A-316 binding module at amino acid residues ALA381, GLN383, GLN404, LEU405 (acceptor), LYS343 (donor), MET378, PRO382, SER342 (donor), THR316, THR344, TRP341, and/or TYR401 (acceptor).

Pharmaceutical compositions of the present disclosure comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the disclosure may comprise different types of carriers depending on whether it is to be administered in solid, liquid or aerosol form, and whether it need to be sterile for such routes of administration as injection. The presently provided compounds can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain aspects, pharmaceutical compositions may comprise, for example, at least about 0.1% of a compound of the present disclosure. In other aspects, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In aspects where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other aspects, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain aspects the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain aspects the candidate substance is prepared for administration by such routes as oral ingestion. In these aspects, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain aspects, carriers for oral administration comprise inert diluents, assimilable edible carriers or combinations thereof. In other aspects of the disclosure, the oral composition may be prepared as a syrup or elixir. A syrup or elixir, and may comprise, for example, at least one active agent, a sweetening agent, a preservative, a flavoring agent, a dye, a preservative, or combinations thereof.

In certain aspects an oral composition may comprise one or more binders, excipients, disintegration agents, lubricants, flavoring agents, and combinations thereof. In certain aspects, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain aspects, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg compound.

In particular aspects, prolonged absorption of an injectable composition can be brought about by the use in the compositions of agents delaying absorption, such as, for example, aluminum monostearate, gelatin, or combinations thereof.

TABLE 1
Exemplary Amino Acid and Nucleic Acid Sequences

| | | SEQ |
|---|---|---|
| Protein | Sequence | ID NO: |
| NSUN1 | MGRKLDPTKEKRGPGRKARKQKGAETELVRFLPAVSDENSKRLSSR | 1 |
| (NOP2), | ARKRAAKRRLGSVEAPKTNKSPEAKPLPGKLPKGISAGAVQTAGKK | |
| human 812 | GPQSLFNAPRGKKRPAPGSDEEEEEEDSEEDGMVNHGDLWGSEDDA | |
| AA isoform | DTVDDYGADSNSEDEEEGEALLPIERAARKQKAREAAAGIQWSEEE | |
| | TEDEEEEKEVTPESGPPKVEEADGGLQINVDEEPFVLPPAGEMEQD | |
| | AQAPDLQRVHKRIQDIVGILRDFGAQREEGRSRSEYLNRLKKDLAI | |
| | YYSYGDFLLGKLMDLFPLSELVEFLEANEVPRPVTLRTNTLKTRRR | |
| | DLAQALINRGVNLDPLGKWSKTGLVVYDSSVPIGATPEYLAGHYML | |
| | QGASSMLPVMALAPQEHERILDMCCAPGGKTSYMAQLMKNTGVILA | |
| | NDANAERLKSVVGNLHRLGVTNTIISHYDGRQFPKVVGGFDRVLLD | |
| | APCSGTGVISKDPAVKTNKDEKDILRCAHLQKELLLSAIDSVNATS | |
| | KTGGYLVYCTCSITVEENEWVVDYALKKRNVRLVPTGLDFGQEGFT | |
| | RFRERRFHPSLRSTRRFYPHTHNMDGFFIAKFKKFSNSIPQSQTGN | |
| | SETATPTNVDLPQVIPKSENSSQPAKKAKGAAKTKQQLQKQQHPKK | |
| | ASFQKLNGISKGADSELSTVPSVTKTQASSSFQDSSQPAGKAEGIR | |
| | EPKVTGKLKQRSPKLQSSKKVAFLRQNAPPKGTDTQTPAVLSPSKT | |
| | QATLKPKDHHQPLGRAKGVEKQQLPEQPFEKAAFQKQNDTPKGPQP | |
| | PTVSPIRSSRPPPAKRKKSQSRGNSQLLLS | |
| NSUN2, | MGRRSRGRRLQQQQRPEDAEDGAEGGGKRGEAGWEGGYPEIVKENK | 2 |
| human 767 | LFEHYYQELKIVPEGEWGQFMDALREPLPATLRITGYKSHAKEILH | |
| AA isoform | CLKNKYFKELEDLEVDGQKVEVPQPLSWYPEELAWHTNLSRKILRK | |
| | SPHLEKFHQFLVSETESGNISRQEAVSMIPPLLLNVRPHHKILDMC | |
| | AAPGSKTTQLIEMLHADMNVPFPEGFVIANDVDNKRCYLLVHQAKR | |
| | LSSPCIMVVNHDASSIPRLQIDVDGRKEILFYDRILCDVPCSGDGT | |
| | MRKNIDVWKKWTTLNSLQLHGLQLRIATRGAEQLAEGGRMVYSTCS | |
| | LNPIEDEAVIASLLEKSEGALELADVSNELPGLKWMPGITQWKVMT | |
| | KDGQWFTDWDAVPHSRHTQIRPTMFPPKDPEKLQAMHLERCLRILP | |
| | HHQNTGGFFVAVLVKKSSMPWNKRQPKLQGKSAETRESTQLSPADL | |
| | TEGKPTDPSKLESPSFTGTGDTEIAHATEDLENNGSKKDGVCGPPP | |
| | SKKMKLFGFKEDPFVFIPEDDPLFPPIEKFYALDPSFPRMNLLTRT | |
| | TEGKKRQLYMVSKELRNVLLNNSEKMKVINTGIKVWCRNNSGEEFD | |
| | CAFRLAQEGIYTLYPFINSRIITVSMEDVKILLTQENPFFRKLSSE | |
| | TYSQAKDLAKGSIVLKYEPDSANPDALQCPIVLCGWRGKASIRTFV | |
| | PKNERLHYLRMMGLEVLGEKKKEGVILTNESAASTGQPDNDVTEGQ | |
| | RAGEPNSPDAEEANSPDVTAGCDPAGVHPPR | |
| 5BI- | cctatctttactcgcgtccaattagaattctacgaattcatc | 3 |
| ECORI-1 | | |
| 5BI- | aatcttaagatgcttaagtagtagttcattccaatcgactct | 4 |
| ECORI-1 | | |
| 5BioTEG- | ttagaattctacgaattcatc | 5 |
| ECORI-1 | | |
| 5IBQ- | tctcagctaaccttacttgatgatgaattcgtagaattctaa | 6 |
| ECORI-1 | | |

TABLE 2
Exemplary identified positive hits from
screening of rationally designed NSUN1/2-
targeting compound libraries in the monocytic
leukemia cell line SC.

| | | Described |
|---|---|---|
| Compound | | activity in |
| IDs | InChlKey | cancers? |
| NSUN1-17 | YWTBGJGMTBHQTM-IBGZPJMESA-N | AKT |
| | | inhibitors. |
| NSUN1-9 | JDUBGYFRJFOXQC-KRWDZBQOSA-N | AKT |
| | | inhibitors. |
| NSUN1-24 | ERILFFEDVVFMT-UHFFFAOYSA-N | No. |
| NSUN1-27 | AOFWXAWCWYRTEA-UHFFFAOYSA-N | Yes. |
| NSUN1-74 | VAMUTLPKXHRDSS-UHFFFAOYSA-N | No. |
| NSUN1-73 | BPNUQXPIQBZCMR-IBGZPJMESA-N | PDK1, FLT1, |
| | | KDR, AUC, |
| | | MAP. |
| NSUN1-67 | FWCUWVSBUUZOEN-UHFFFAOYSA-N | No. |
| NSUN1-31 | XAABVJIUUFRECX-UHFFFAOYSA-N | No. |
| NSUN1-64 | ZAKYLTYTJKXKMH-UHFFFAOYSA-N | No. |
| NSUN1-25 | UBFPBRMXXWEZQY-UHFFFAOYSA-N | USP |
| NSUN1-30 | ITFMNJKUPKOXBA-UHFFFAOYSA-N | No. |
| NSUN1-11 | WSEKTEUGRLFBSE-UHFFFAOYSA-N | No. |
| NSUN1-54 | YEBWEMIJOWUBEM-UHFFFAOYSA-N | No. |
| NSUN1-10 | VGGGPCQERPFHOB-RDBSUJKOSA-N | Bestatin or |
| | | Ubenimex for |
| | | ATLL, K562, |
| | | MDS. |
| NSUN2-10 | AHYKNYRHFWASSX-UHFFFAOYSA-N | No. |
| NSUN2-3 | YIQZXGYCBIVEOQ-UHFFFAOYSA-N | No. |
| NSUN2-11 | WAHCRKCBUNYAFU-UHFFFAOYSA-N | No. |
| NSUN2-39 | DFMUOKBJQQMPGY-UHFFFAOYSA-N | No. |
| NSUN2-98 | IVFXWRBLNQJPOG-UHFFFAOYSA-N | No. |
| NSUN2-19 | HNJQWJLYYSSLKV-UHFFFAOYSA-N | No. |
| NSUN2-20 | MSIJWQAFFGMGRI-UHFFFAOYSA-N | No. |
| NSUN2-21 | PURCNHQUXMNILH-UHFFFAOYSA-N | No. |
| NSUN2-38 | KKWXISYKQWZSJS-UHFFFAOYSA-N | No. |
| NSUN2-64 | FVSBCUOXYSPVHT-UHFFFAOYSA-N | No. |
| NSUN2-18 | CIHIIVOLVMDQHP-UHFFFAOYSA-N | No. |
| NSUN2-17 | IMQQSPPTTVDZDA-UHFFFAOYSA-N | No. |
| NSUN2-37 | JERGSIUBSIEOAR-UHFFFAOYSA-N | No. |

In some aspects, NSUN1-17 has a high-efficacy of tumor cell growth inhibition in multiple leukemia lines and/or lung cancer cell lines.

In some aspects, synergy experiments indicate that besides the NSUN1 A-460 binding module, NSUN1-17 is likely to target multiple binding modules in both NSUN1 and NSUN2. Without being limited by theory, this may potentially be why NSUN1-17 is effective in multiple different cancer types.

In some aspects, a small molecule inhibitor of NSUN1/2-ACS interactions is not NSUN1-17, NSUN1-17.13, NSUN1-17.16, NSUN1-9, NSUN1-24, NSUN1-27, NSUN1-74, NSUN1-73, NSUN1-31, NSUN1-64, NSUN1-67, NSUN1-25, NSUN1-30, NSUN1-11, NSUN1-54, NSUN1-10, NSUN2-10, NSUN2-3, NSUN2-11, NSUN2-39, NSUN2-98, NSUN2-19, NSUN2-20, NSUN2-21, NSUN2-38, NSUN2-64, NSUN2-18, NSUN2-17, NSUN2-37, and/or NSUN2-35.

In some aspects, a small molecule inhibitor of NSUN1/2-ACS interactions is one or more (e.g., 1, 2, 3, 4, 5, 6, 7, 8, 9, 10, 11, 12, 13, 14, 15, etc.) molecules selected from the group comprising NSUN1-17, NSUN1-17.13, NSUN1-17.16, NSUN1-9, NSUN1-24, NSUN1-27, NSUN1-74, NSUN1-73, NSUN1-31, NSUN1-64, NSUN1-67, NSUN1-25, NSUN1-30, NSUN1-11, NSUN1-54, NSUN1-10, NSUN2-10, NSUN2-3, NSUN2-11, NSUN2-39, NSUN2-98, NSUN2-19, NSUN2-20, NSUN2-21, NSUN2-38, NSUN2-64, NSUN2-18, NSUN2-17, NSUN2-37, and NSUN2-35.

In some aspects, provided herein are compounds with at least dual specificities for binding modules in both NSUN1 and NSUN2. In some aspects, methods and compositions described herein comprise binding of 0, 1, 2, 3, 4, 5, 6, 7, 8, 9, 10, 11, 12, or 13 binding modules in NSUN1 and/or NSUN2. In certain aspects, methods and compositions provided herein bind to at least, exactly, or no more than 0, 1, 2, 3, 4, 5, 6, 7, 8, 9, 10, 11, 12, or 13 binding modules in NSUN1 and/or NSUN2. In certain aspects, compositions provided herein interact with NSUN1 and/or NSUN2 binding modules as described in Table 4, wherein the notation “X”/400 denotes the relative binding strength of the noted molecule in comparison to the other molecules identified.

In some aspects, NSUN1 (SEQ ID NO: 2) can be characterized as comprising 4 binding modules, characterized as Module 1 A219 (FIG. 10G); Module 2 A316 (FIG. 10A); Module 3 A371 (FIG. 10H); and Module 4 A-460 (FIG. 10B).

In certain aspects, NSUN1 Module 1 A-219 comprises a binding pocket residue finger print including potential contact/interaction (e.g., acceptor, donor, contact) with amino acid residues ALA255, ARG 242, ARG257, ASP245, GLN256, GLU217, GLU259, ILE249, PHE219, PR0222, VAL220, ARG238, ARG251, ASP215, GLN244, GLU216, GLU258, GLY248, LYS241, PR0218, and/or VAL214. In some aspects, a compound provided herein interacts with 0, 1, 2, 3, 4, 5, 6, 7, 8, 9, 10, 11, 12, 13, 14, 15, 16, 17, 18, 19, 20, or 21 of the noted amino acid residues.

In certain aspects, NSUN1 Module 2 A-316 comprises a binding pocket residue finger print including potential contact/interaction (e.g., acceptor, donor, contact) with amino acid residues ALA381, ARG321, GLN383, GLU384, LEU317, LEU544, LYS407, PR0382, THR316, THR344, TYR401, ARG320, ARG322, GLN404, GLY543, LEU405, LYS343, MET378, SER342, THR319, and/or TRP341. In some aspects, a compound provided herein interacts with 0, 1, 2, 3, 4, 5, 6, 7, 8, 9, 10, 11, 12, 13, 14, 15, 16, 17, 18, 19, 20, or 21 of the noted amino acid residues.

In certain aspects, NSUN1 Module 3 A-371 comprises a binding pocket residue finger print including potential contact/interaction (e.g., acceptor, donor, contact) with amino acid residues ALA371, ALA394, ARG308, ARG421, ASN575, ASP336, ASP460, ASP472, ASP577, CYS517, GLN369, GLY370, GLY397, HIS574, LEU338, LEU368, LYS398, LYS471, MET367, MET576, PHE579, PR0307, PR0309, PR0337, PR0395, SER372, SER373, SER470, THR311, TRP341, TYR349, VAL310, VAL347, and/or VAL468. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 of the noted amino acid residues.

In certain aspects, NSUN1 Module 4 A-460 comprises a binding pocket residue finger print including potential contact/interaction (e.g., acceptor, donor, contact) with amino acid residues ALA394, ALA417, ALA461, ARG421, ASN418, ASP416, ASP460, ASP472, ASP577, CYS392, CYS463, CYS517, GLN369, GLY370, GLY397, GLY465, LEU368, LEU490, LEU494, LYS398, LYS471, LYS479, MET367, PHE579, PR0395, PR0462, SER372, SER373, SER464, THR311, TYR361, VAL310, VAL468, and/or VAL475. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 of the noted amino acid residues.

In some aspects, NSUN2 (SEQ ID NO: 1) can be characterized as comprising 9 binding modules, characterized as Module 1 A89 (FIG. 10J); Module 2 A162 (FIG. 10K); Module 3 A202 (FIG. 10L); Module 4 A271 (FIG. 10E); Module 5 A279 (FIG. 10F); Module 6 A301 (FIG. 10I); Module 7 A580 (FIG. 10C); Module 8 A691 (FIG. 10D); and Module 9 A700 (FIG. 10M).

In certain aspects, NSUN2 Module 1 A-89 comprises a binding pocket residue finger print including potential contact/interaction (e.g., acceptor, donor, contact) with amino acid residues ALA670, ARG700, ASN96, ASP669, CYS673, CYS93, GLN672, GLU123, GLU152, GLU625, GLU89, GLY703, HIS92, ILE90, LEU671, LEU91, LYS628, LYS84, LYS88, LYS95, LYS97, MET202, MET624, MET701, MET702, PR0668, PR0674, SER85, TYR609, and/or TYR661. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the noted amino acid residues.

In certain aspects, NSUN2 Module 2 A-162 comprises a binding pocket residue finger print including potential contact/interaction (e.g., acceptor, donor, contact) with amino acid residues ARG160, ARG278, ARG411, ASN280, ASN418, ASP282, ASP397, GLN161, GLU162, GLY420, HIS415, ILE281, LYS279, LYS396, LYS400, PHE393, PR0390, PR0394, PR0395, PRO414, SER189, THR276, and/or THR419. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, or 24, of the noted amino acid residues.

In certain aspects, NSUN2 Module 3 A-202 comprises a binding pocket residue finger print including potential contact/interaction (e.g., acceptor, donor, contact) with amino acid residues ASN203, ASP201, CYS673, GLN672, GLU662, HIS199, HIS86, LYS660, LYS84, LYS88, MET202, PR0205, PR0234, PR0613, PR0663, PR0674, SER232, SER85, THR610, TYR609, TYR612, TYR83, VAL204, and/or VAL676. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, or 24, of the noted amino acid residues.

In certain aspects, NSUN2 Module 4 A-271 comprises a binding pocket residue finger print including potential contact/interaction (e.g., acceptor, donor, contact) with amino acid residues ALA185, ALA186, ALA243, ARG220, ARG278, ASN291, ASP215, ASP217, ASP242, ASP268, ASP282, CYS184, CYS271, CYS321, GLN294, GLY273, ILE302, LEU295, LEU298, LYS279, LYS285, MET277, PHE422, PR0270, SER272, THR320, TRP284, TRP287, VAL216, and/or VAL269. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the noted amino acid residues.

In certain aspects, NSUN2 Module 5 A-279 comprises a binding pocket residue finger print including potential contact/interaction (e.g., acceptor, donor, contact) with amino acid residues ALA186, ALA76, ARG133, ARG160, ARG220, ARG230, ARG278, ASN130, ASN280, ASP215, ASP268, CYS184, CYS271, CYS321, GLN161, GLN227, GLU162, GLY156, ILE281, LEU131, LEU136, LEU149, LEU231, LEU74, LEU78, LYS134, LYS190, LYS279, LYS43, PHE145, PHE422, PRO187, PR0270, PR073, PRO75, SER132, SER159, SER165, SER189, THR129, THR153, THR192, THR276, THR77, THR81, and/or VAL269. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 of the noted amino acid residues.

In certain aspects, NSUN2 Module 6 A-301 comprises a binding pocket residue finger print including potential contact/interaction (e.g., acceptor, donor, contact) with amino acid residues ARG301, ARG305, ARG389, ASP242, ASP378, CYS502, GLU308, GLU337, GLY297, GLY500, GLY503, HIS385, ILE246, LEU300, LYS338, LYS396, PHE393, PR0390, PR0394, PR0395, PR0504, PRO505, SER244, SER245, SER334, SER507, THR304, THR386, THR391, TRP377A, and/or VAL501. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the noted amino acid residues.

In certain aspects, NSUN2 Module 7 A-580 comprises a binding pocket residue finger print including potential contact/interaction (e.g., acceptor, donor, contact) with amino acid residues ALA761, ARG551, ASN218, ASN240, ASN582, ASP518, ASP759, CYS758, GLU517, GLU576, ILE581, LEU223, LEU512, LEU571, LEU572, LYS219, LYS229, LYS511, LYS516, LYS579, MET510, MET578, PHE513, PHE515, PHE520, PHE522, PRO519, PR0760, SER575, TYR222, VAL225, VAL521, and/or VAL580. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 of the noted amino acid residues.

In certain aspects, NSUN2 Module 8 A-691 comprises a binding pocket residue finger print including potential contact/interaction (e.g., acceptor, donor, contact) with amino acid residues ALA200, ARG230, ARG79, ASN157, ASN693, GLN193, GLN605, GLU123, GLU154, GLU196, GLU606, GLU694, GLY156, GLY82, HIS199, HIS226, HIS697, HIS86, ILE195, ILE80, LEU125, LEU170, LEU231, LYS229, LYS84, MET197, PR0691, SER155, SER232, SER233, THR81, TYR121, TYR609, TYR698, and/or TYR83. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 of the noted amino acid residues.

In certain aspects, NSUN2 Module 9 A-700 comprises a binding pocket residue finger print including potential contact/interaction (e.g., acceptor, donor, contact) with amino acid residues ARG700, ASN693, CYS93, GLN147, GLU143, GLU154, GLU705, GLU709, GLY703, GLY708, HIS141, HIS146, LEU696, LEU704, LEU707, LYS144, LYS692, LYS97, PHE148, PRO140, SER151, TYR98, VAL150, and/or VAL706. In some aspects, a compound provided herein interacts with 0, 1, 2, 3, 4, 5, 6, 7, 8, 9, 10, 11, 12, 13, 14, 15, 16, 17, 18, 19, 20, 21, 22, 23, or 24 of the noted amino acid residues.

In some aspects, NSUN1-14 and/or NSUN1-73 have high-efficacy of tumor cell growth inhibition in multiple leukemia cell lines and/or lung cancer cell lines.

In some aspects, provided herein are modified (e.g., derivatized) NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-11, NSUN2-37, and/or NSUN2-35 compounds.

In some aspects, provided herein are water soluble derivatives of NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-11, NSUN2-37, and/or NSUN2-35 compounds.

In some aspects, provided herein are modified (e.g., derivatized) NSUN1-17.16, NSUN1-11, NSUN1-67, NSUN2-11, NSUN2-37, and/or NSUN2-35 compounds.

In some aspects, provided herein are water soluble derivatives of NSUN1-17.16, NSUN1-11, NSUN1-67, NSUN2-11, NSUN2-37, and/or NSUN2-35 compounds.

TABLE 3
Identified positive hits
actively undergoing derivatization.

| Compound | | |
|---|---|---|
| IDs | InChlKey | SMILES |
| NSUN1-24 | XHJVBQVXHIAWRW- | C1(C2C═CC═CC═2NC═1C)C1═CSC(NC |
| | UHFFFAOYSA-N | (CSCC(O)═O)═O)═N1 |
| NSUN1-11 | WSEKTEUGRLFBSE- | C1═CC═NC(═C1)OCC2═CC═C(C═C2) |
| | UHFFFAOYSA-N | CC3═NOC(═C3)C4═C(N═CC═C4)N |
| NSUN1-14 | IDRGFNPZDVBSSE- | C(NC1═CC═CC(C2C3C(C═CC═2)═CN═C |
| | UHFFFAOYSA-N | (NC2═CC═C(N4CCN(CCO)CC4)C(F)═C2F) |
| | | N═3)═C1)(═O)C═C |
| NSUN1-16 | NZNTWOVDIXCHHS- | O═C(N)C1C(NC2C═C(C)C═CC═2)═NC |
| | LSDHHAIUSA-N | (N[C@@H]2CCCC[C@@H]2N)═NC═1 |
| NSUN1-17 | YWTBGJGMTBHQTM- | N[C@@H](CC1C2C═CC═CC═2NC═1) |
| | IBGZPJMESA-N | COC1C═NC═C(C2C═CC3C(═C(NN═3) |
| | | C)C═2)C═1 |
| NSUN1- | WJIPVGBHPKGRQC- | C1(═CC2C(═CC═CC═2OC)N1CC)C1═NNC |
| 17.3 | UHFFFAOYSA-N | (NC(═O)C2C═CC(═C(OC)C═2)OC)═C1 |
| NSUN1- | BTZHUVVIKAXMQR- | CC(C)CC1═NC(═NN1)NC(═O)C2═CC(═NN2) |
| 17.16 | UHFFFAOYSA-N | C3═CC4═C(C═C3)OCC4 |
| NSUN1-19 | OFLOSUNRPFWODP- | [H]Cl.NC1(CCN(C2N═CN═C3NC═CC═23) |
| | UHFFFAOYSA-N | CC1)CC1C═CC(C1)═CC═1 |
| NSUN1-64 | ZAKYLTYTJKXKMH- | COC(═O)C1═CC═C(C═C1) |
| | UHFFFAOYSA-N | CSC2═NC3═CC═CC═C3C(═O)N2 |
| NSUN1-67 | FWCUWVSBUUZOEN- | C1═CC(═CC═C1NC2═NC3═NC═CC(═C3C |
| | UHFFFAOYSA-N | (═O)N2)C4═CC═NC═C4)C |
| NSUN1-73 | BPNUQXPIQBZCMR- | C(OC1═CC(C2C═CC3═C(C═2)C |
| | IBGZPJMESA-N | (C)═NN3)═CN═C1)[C@@H](N) |
| | | CC1═CC═CC═C1 |
| NSUN1-74 | VAMUTLPKXHRDSS- | C1CCN2C(═NN═C2C3═CC(═CC═C3)NC(═O) |
| | UHFFFAOYSA-N | C4═CC(═NN4)C5═CC═CC═C5)C1 |
| NSUN2-11 | WAHCRKCBUNYAFU- | CC1═CC2═C(C═C1)N═C(N2)CCNC(═O) |
| | UHFFFAOYSA-N | C3═C4CCC5═C(C4═NN3)C═CC(═C5)OC |
| NSUN2-37 | JERGSIUBSIEOAR- | C1CCN(CC1)CC2═CC═C(C═C2)NC(═O) |
| | UHFFFAOYSA-N | C3═CC═CC(═C3)COC4═CC═C(C═C4)Br |
| NSUN2-35 | ZKIAIYBUSXZPLP- | C1CN(CCN1CCCCOC2═CC3═C(C═C2)C═CC |
| | UHFFFAOYSA-N | (═O)N3)C4═C5C═CSC5═CC═C4 |

In some aspects, inhibition of NSUN1 and ACS activity is most effectively inhibited by targeting the binding modules A-316 and/or A-460 of NSUN1.

In some aspects, inhibition of NSUN2 and ACS activity is most effectively inhibited by targeting the binding modules A-580 and/or A-691 of NSUN2. In some aspects, inhibition of NSUN2 and ACS activity is most effectively inhibited by targeting the binding modules A-271 and/or A-279 of NSUN2. In some aspects, inhibition of NSUN2 and ACS activity is most effectively inhibited by targeting the binding modules A-271, A-279, A-580, and/or A-691 (see e.g., FIGS. 10A-10F).

In some aspects, inhibition of NSUN1/2 and ACS activity is most effectively inhibited by targeting multiple binding modules in both NSUN1 and NSUN2.

In some aspects, a small molecule NSUN1/2-ACS inhibitor targets multiple modules in both NSUN1 and NSUN2. In some aspects, a small molecule NSUN1/2-ACS inhibitor does not target only one module with high affinity.

In some aspects, screening for small molecule NSUN1/2-ACS inhibitors comprises utilization of human acute myeloid/monocytic leukemia cell lines (e.g., SC) and/or human lung cancer cell lines (e.g., H82), both of which are venetoclax-resistant cell lines.

In some aspects, small molecule NSUN1/2-ACS inhibitors are effective for inhibiting growth of acute myeloid leukemia cells (e.g., mouse cell line C1498).

In some aspects, small molecule NSUN1/2-ACS inhibitors are effective for inhibiting growth of lung cancer cells (e.g., mouse cell line LL/2).

In some aspects, small molecule NSUN1/2-ACS inhibitors are effective for inhibiting growth of cancer cells.

In some aspects, provided herein are small-molecule compounds and methods of using the same for inhibition of the growth of drug-resistant cancer cells. In some aspects, provided herein are small-molecule compounds and methods of using the same for inhibition of the growth of drug-resistant myeloid leukemia cells.

In some aspects, provided herein are compounds that can effectively disrupt the NSUN1/2-mediated drug-resistant active chromatin structures (ACS) associated with drug-resistant cancer cells.

In some aspects, the in vivo efficacy of compounds described herein are verified in syngeneic C1498 and/or LL/2 CDX mouse models. In some aspects, the in vivo efficacy of compounds described herein are verified in syngeneic SC and/or H82 mouse models.

TABLE 4
Exemplary binding module rankings

| Compound | Targeted | Binding modules/ | Binding modules/ | |
|---|---|---|---|---|
| ID | Libraries | ranks in NSUN1 | ranks in NSUN2 | InChIKey |
| NSUN1-11 | NSUN1 | NSUN1 A-219 (111/400); | NSUN2 A-700 (176/400); | WSEKTEUGRLFBSE- |
| | | NSUN1 A-460 (17/400); | NSUN2 A-202 (206/400); | UHFFFAOYSA-N |
| | | | NSUN2 A-691 (303/400) | |
| NSUN1-67 | NSUN1 | None | None | FWCUWVSBUUZOEN- |
| | | | | UHFFFAOYSA-N |
| NSUN1-19 | NSUN1 | NSUN1 A-460 (29/400) | NSUN2 A-301 (361/400); | OFLOSUNRPFWODP- |
| | | | NSUN2 A-700 (251/400); | UHFFFAOYSA-N |
| | | | NSUN2 A-89 (179/400); | |
| | | | NSUN2 A-202 (212/400) | |
| NSUN1-22 | NSUN1 | None | None | CSJIETGIQHJQTG- |
| | | | | UHFFFAOYSA-N |
| NSUN1-74 | NSUN1 | NSUN1 A-219 (295/400); | NSUN2 A-279 (396/400); | VAMUTLPKXHRDSS- |
| | | NSUN1 A-460 (28/400) | NSUN2 A-691 (240/400); | UHFFFAOYSA-N |
| | | | NSUN2 A-271 (105/400) | |
| NSUN1-17 | NSUN1 | NSUN1 A-316 (7/400); | NSUN2 A-89 (88/400); | YWTBGJGMTBHQTM- |
| | | NSUN1 A-219 (325/400); | NSUN2 A-279 (46/400); | IBGZPJMESA-N |
| | | NSUN1 A-460 (30/400); | NSUN2 A-691 (132/400); | |
| | | NSUN1 A-371 (154/400) | NSUN2 A-580 (5/400); | |
| | | | NSUN2 A-271 (11/400); | |
| | | | NSUN2 A-162 (34/400) | |
| NSUN1-73 | NSUN1 | NSUN1 A-316 (10/400); | NSUN2 A-89 (38/400); | BPNUQXPIQBZCMR- |
| | | NSUN1 A-219 (90/400); | NSUN2 A-202 (259/400); | IBGZPJMESA-N |
| | | NSUN1 A-460 (3/400); | NSUN2 A-691 (328/400); | |
| | | NSUN1 A-371 (72/400) | NSUN2 A-580 (1/400); | |
| | | | NSUN2 A-271 (34/400); | |
| | | | NSUN2 A-162 (243/400) | |
| NSUN1-9 | NSUN1 | None | None | JDUBGYFRJFOXQC- |
| | | | | KRWDZBQOSA-N |
| NSUN1-14 | NSUN1 | NSUN1 A-460 (23/400) | NSUN2 A-202 (202/400); | IDRGFNPZDVBSSE- |
| | | | NSUN2 A-580 (175/400) | UHFFFAOYSA-N |
| NSUN1-16 | NSUN1 | NSUN1 A-316 (261/400); | NSUN2 A-162 (232/400) | NZNTWOVDIXCHHS- |
| | | NSUN1 A-460 (11/400) | | LSDHHAIUSA-N |
| NSUN1-17.16 | NSUN1 | | | BTZHUVVIKAXMQR- |
| | | | | UHFFFAOYSA-N |
| NSUN2-11 | NSUN2 | NSUN1 A460 (42/400); | NSUN2 A301 (187/400); | WAHCRKCBUNYAFU- |
| | | NSUN1 A219 (148/400); | NSUN2 A-89 (228/400); | UHFFFAOYSA-N |
| | | NSUN1 A-316 (205/400) | NSUN2 A-279 (143/400); | |
| | | | NSUN2 A-691 (4/400); | |
| | | | NSUN2 A-580 (145/400); | |
| | | | NSUN2 A-271 (74/400); | |
| | | | NSUN2 A-162 (56/400) | |
| NSUN2-37 | NSUN2 | None | NSUN2 A301 (331/400); | JERGSIUBSIEOAR- |
| | | | NSUN2 A-691 (2/400); | UHFFFAOYSA-N |
| | | | NSUN2 A-162 (364/400) | |
| NSUN2-35 | NSUN2 | None | None | ZKIAIYBUSXZPLP- |
| | | | | UHFFFAOYSA-N |
| NSUN2-34 | NSUN2 | None | None | JDUBGYFRJFOXQC- |
| | | | | KRWDZBQOSA-N |
| NSUN2-98 | NSUN2 | None | NSUN2 A301 (398/400); | IVFXWRBLNQJPOG- |
| | | | NSUN2 A-691 (3/400) | UHFFFAOYSA-N |
| NSUN2-38 | NSUN2 | None | NSUN2 A-691 (11/400) | KKWXISYKQWZSJS- |
| | | | | UHFFFAOYSA-N |
| NSUN2-39 | NSUN2 | NSUN1 A-460 (307/400); | NSUN2 A301 (331/400); | DFMUOKBJQQMPGY- |
| | | NSUN1 A-219 (256/400) | NSUN2 A700 (284/400); | UHFFFAOYSA-N |
| | | | NSUN2 A89 (20/400); | |
| | | | NSUN2 A691 (12/400); | |
| | | | NSUN2 A-271 (299/400) | |
| NSUN2-67 | NSUN2 | None | None | WUSKNQFPTJWRBS- |
| | | | | UHFFFAOYSA-N |
| NSUN2-19 | NSUN2 | NSUN1 A-219 (208/400); | NSUN2 A279 (22/400); | HNJQWJLYYSSLKV- |
| | | NSUN1 A-316 (50/400) | NSUN2 A-202 (183/400); | UHFFFAOYSA-N |
| | | | NSUN2 A-691 (13/400) | |
| NSUN2-20 | NSUN2 | NSUN1 A-219 (213/400) | NSUN2 A279 (163/400); | MSIJWQAFFGMGRI- |
| | | | NSUN2 A-691 (6/400) | UHFFFAOYSA-N |
| NSUN2-21 | NSUN2 | None | NSUN2 A-691 (10/400) | PURCNHQUXMNILH- |
| | | | | UHFFFAOYSA-N |
| NSUN2-64 | NSUN2 | None | NSUN2 A-691 (1/400) | |

TABLE 5
Exemplary results for verified NSUN1-ACS inhibitors

| Compound | Targeted | Effective | | |
|---|---|---|---|---|
| ID | Libraries | cell lines | Animal models | InChIKey |
| NSUN1-17.16 | NSUN1 | SC, C1498, H82 | syngeneic (1st | BTZHUVVIKAXMQR- |
| | | | tier, on-going) | UHFFFAOYSA-N |
| NSUN1-11 | NSUN1 | SC, C1498, H82 | syngeneic (1st | WSEKTEUGRLFBSE- |
| | | | tier, on-going) | UHFFFAOYSA-N |
| NSUN1-67 | NSUN1 | SC, C1498 & | syngeneic (1st | FWCUWVSBUUZOEN- |
| | | H82 | tier, on-going) | UHFFFAOYSA-N |
| NSUN1-19 | NSUN1 | H82 > SC & | TBD | OFLOSUNRPFWODP- |
| | | C1498 | | UHFFFAOYSA-N |
| NSUN1-22 | NSUN1 | SC & H82, | TBD | CSJIETGIQHJQTG- |
| | | C1498 (weakly) | | UHFFFAOYSA-N |
| NSUN1-74 | NSUN1 | SC & H82, | TBD | VAMUTLPKXHRDSS- |
| | | C1498 (weakly) | | UHFFFAOYSA-N |
| NSUN1-17* | NSUN1 | SC, H82, C1498 | TBD | YWTBGJGMTBHQTM- |
| | | | | IBGZPJMESA-N |
| NSUN1-73* | NSUN1 | SC, H82, C1498 | TBD | BPNUQXPIQBZCMR- |
| | | | | IBGZPJMESA-N |
| NSUN1-9 | NSUN1 | SC & C1498 > | TBD | JDUBGYFRJFOXQC- |
| | | H82 | | KRWDZBQOSA-N |
| NSUN1-14* | NSUN1 | H82 & C1498 > | TBD | IDRGFNPZDVBSSE- |
| | | SC | | UHFFFAOYSA-N |
| NSUN1-16* | NSUN1 | H82 & C1498, | TBD | NZNTWOVDIXCHHS- |
| | | but not SC | | LSDHHAIUSA-N |

SC: Venetoclax-resistant human monocytic leukemia cell line. H82: Venetoclax-resistant human lung cancer cell line. C1498: Mouse myeloid leukemia cell line.

TABLE 6
Exemplary results for verified NSUN2-ACS inhibitors

| | | | | Known or | |
|---|---|---|---|---|---|
| | | | | patented | |
| Compound | Targeted | Effective | | anticancer | |
| ID | Libraries | cell lines | Animal models | activities | InChIKey |
| NSUN2-11 | NSUN2 | SC, C1498, H82 | syngeneic (1st | No. | WAHCRKCBUNYAFU- |
| | | | tier, on-going) | | UHFFFAOYSA-N |
| NSUN2-37 | NSUN2 | H82 & SC > | syngeneic (1st | No | JERGSIUBSIEOAR- |
| | | C1498 | tier, on-going) | | UHFFFAOYSA-N |
| NSUN2-35 | NSUN2 | H82, C1498, | syngeneic (1st | No. antipsychotic | ZKIAIYBUSXZPLP- |
| | | but not in SC | tier, on-going) | agent | UHFFFAOYSA-N |
| NSUN2-34 | NSUN2 | H82, C1498, | TBD | Yes | JDUBGYFRJFOXQC- |
| | | but not in SC | | | KRWDZBQOSA-N |
| NSUN2-98 | NSUN2 | SC > H82 & | TBD | No | IVFXWRBLNQJPOG- |
| | | C1498 | | | UHFFFAOYSA-N |
| NSUN2-38 | NSUN2 | SC, no in | TBD | No | KKWXISYKQWZSJS- |
| | | H82 & C1498 | | | UHFFFAOYSA-N |
| NSUN2-39 | NSUN2 | SC > C1498 | TBD | No | DFMUOKBJQQMPGY- |
| | | & H82 | | | UHFFFAOYSA-N |
| NSUN2-67 | NSUN2 | Weakly in SC, | TBD | No | WUSKNQFPTJWRBS- |
| | | H82 & C1498 | | | UHFFFAOYSA-N |
| NSUN2-19 | NSUN2 | H82, no in | TBD | No | HNJQWJLYYSSLKV- |
| | | SC & C1498 | | | UHFFFAOYSA-N |
| NSUN2-20 | NSUN2 | H82, no in | TBD | No | MSIJWQAFFGMGRI- |
| | | SC & C1498 | | | UHFFFAOYSA-N |
| NSUN2-21 | NSUN2 | H82, no in | TBD | No | PURCNHQUXMNILH- |
| | | SC & C1498 | | | UHFFFAOYSA-N |

SC: Venetoclax-resistant human monocytic leukemia cell line. H82: Venetoclax-resistant human lung cancer cell line. C1498: Mouse myeloid leukemia cell line.

### REFERENCES

All publications mentioned in the specification are indicative of the level of those skilled in the art to which the disclosure pertains. All patents and publications are herein incorporated by reference to the same extent as if each individual publication was specifically and individually indicated to be incorporated by reference.

What is claimed is:

1. A method of treating a disease in an individual, the method comprising the step of administering to the individual a therapeutically effective amount of any one or more of an inhibitor of NSUN1 and/or NSUN2 interactions with Active Chromatin Site (ACS) associated proteins and/or complexes (NSUN1/2-ACS inhibitor).

2. The method of claim 1, wherein the NSUN1/2-ACS inhibitor comprises NSUN2-11, NSUN2-37, NSUN2-35, NSUN1-17.16, NSUN1-11, NSUN1-67, NSUN2-34, NSUN2-98, NSUN2-38, NSUN2-39, NSUN1-19, NSUN1-9, NSUN1-10, NSUN1-17, NSUN1-17.13, NSUN1-24, NSUN1-25, NSUN1-27, NSUN1-30, NSUN1-31, NSUN1-54, NSUN1-64, NSUN1-73, NSUN1-74, NSUN2-3, NSUN2-10, NSUN2-17, NSUN2-18, NSUN2-19, NSUN2-20, NSUN2-21, NSUN2-64, and/or a derivative thereof.

3. The method of claim 1 or 2, wherein the NSUN1/2-ACS inhibitor comprises NSUN2-11, NSUN2-37, NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-35, and/or a derivative thereof.

4. The method of claim 3, wherein the NSUN1/2-ACS inhibitor comprises a water soluble derivative of compounds NSUN2-11, NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-37, and/or NSUN2-35.

5. The method of claim 1 or 2, wherein the NSUN1/2-ACS inhibitor comprises NSUN1-24, NSUN1-14, NSUN1-16, NSUN1-17, NSUN1-17.3, NSUN1-19, NSUN1-73, and/or a derivative thereof.

6. The method of claim 5, wherein the NSUN1/2-ACS inhibitor comprises a water soluble derivative of compounds NSUN1-24, NSUN1-14, NSUN1-16, NSUN1-17, NSUN1-17.3, NSUN1-19, and/or NSUN1-73.

7. The method of claim 1 or 2, wherein the NSUN1/2-ACS inhibitor comprises compounds NSUN1-17.16, NSUN1-11, NSUN1-67, NSUN2-11, NSUN2-37, NSUN2-35, and/or a derivative thereof.

8. The method of claim 7, wherein the NSUN1/2-ACS inhibitor comprises a water soluble derivative of compounds NSUN1-17.16, NSUN1-11, NSUN1-67, NSUN2-11, NSUN2-37, and/or NSUN2-35.

9. The method of any one of claims 1-8, wherein the disease is cancer.

10. The method of claim 9, wherein the cancer is a blood cancer or a solid cancer.

11. The method of claim 9 or 10, wherein the cancer is a leukemia and/or a myelodysplastic syndrome (MDS).

12. The method of any one of claims 9-11, wherein the cancer is a drug-resistant cancer.

13. The method of any one of claims 9-12, wherein the cancer is resistant to venetoclax-based and/or 5-azacytidine-based therapies.

14. The method of any one of claims 1-13, wherein the NSUN1/2-ACS inhibitor binds to at least the A-460 binding module of NSUN1.

15. The method of any one of claims 1-14, wherein the NSUN1/2-ACS inhibitor binds to at least the A-316 binding module of NSUN1.

16. The method of any one of claims 1-15, wherein the NSUN1/2-ACS inhibitor binds to at least the A-691 binding module of NSUN2.

17. The method of any one of claims 1-16, wherein the NSUN1/2-ACS inhibitor binds to at least the A-580 binding module of NSUN2.

18. The method of any one of claims 1-17, wherein the NSUN1/2-ACS inhibitor binds to at least the A-271 binding module of NSUN2.

19. The method of any one of claims 1-18, wherein the NSUN1/2-ACS inhibitor binds to at least the A-279 binding module of NSUN2.

20. The method of any one of claims 1-19, wherein the NSUN1/2-ACS inhibitor binds to at least two binding modules of NSUN1 and/or NSUN2 selected from the group consisting of NSUN1 A-460, NSUN1 A-316, NSUN1 A-371, NSUN1 A-219, NSUN2 A-700, NSUN2 A-691, NSUN2 A-580, NSUN2 A-301, NSUN2-A279, NSUN2 A-271, NSUN2 A-202, NSUN2 A-162, and/or NSUN2 A-89.

21. The method of any one of claims 1-20, wherein the NSUN1/2-ACS inhibitor binds to at least two binding modules of NSUN1 and/or NSUN2 selected from the group consisting of NSUN1 A-460, NSUN1 A-316, NSUN2 A-691, NSUN2 A-580, NSUN2 A-271, and NSUN2 A-279.

22. The method of any one of claims 1-21, wherein the NSUN1/2-ACS inhibitor binds to more than two binding modules of NSUN1 and/or NSUN2.

23. The method of any one of claims 14-22, wherein the NSUN1/2-ACS inhibitor binds to at least one binding module of NSUN1 and at least one binding module of NSUN2.

24. The method of any one of claims 1-23, wherein the NSUN1/2-ACS inhibitor comprises, consists essentially of, or consists of NSUN1-11 and/or a derivative thereof.

25. The method of any one of claims 1-23, wherein the NSUN1/2-ACS inhibitor comprises, consists essentially of, or consists of NSUN1-67 and/or a derivative thereof.

26. The method of any one of claims 1-23, wherein the NSUN1/2-ACS inhibitor comprises, consists essentially of, or consists of NSUN1-17.16 and/or a derivative thereof.

27. The method of any one of claims 1-23, wherein the NSUN1/2-ACS inhibitor comprises, consists essentially of, or consists of NSUN2-11 and/or a derivative thereof.

28. The method of any one of claims 1-23, wherein the NSUN1/2-ACS inhibitor comprises, consists essentially of, or consists of NSUN2-37 and/or a derivative thereof.

29. The method of any one of claims 1-23, wherein the NSUN1/2-ACS inhibitor comprises, consists essentially of, or consists of NSUN2-35 and/or a derivative thereof.

30. The method of any one of claims 9-29, wherein the NSUN1/2-ACS inhibitor reduces cancer cell proliferation.

31. The method of any one of claims 1-30, wherein the NSUN1/2-ACS inhibitor has substantively no effect on NSUN1/2-BRD4 interactions, substantively no effect NSUN1/2-m5C interactions, substantively inhibits NSUN1/2-RNAPII interactions, substantively inhibits NSUN1/2-c-MYC interactions, substantively inhibits NSUN1/2-POLRMT interactions, and/or substantively inhibits NSUN1/2-MTIF3 interactions.

32. The method of any one of claims 1-31, wherein the NSUN1/2-ACS inhibitor is part of a combination therapy.

33. The method of claim 32, wherein the combination therapy comprises venetoclax-based and/or 5-azacytidine-based therapies.

34. The method of any one of claims 1-33, wherein the ACS associated complex comprises an elongating RNA-Polymerase-II (eRNAPII) complex.

35. The method of any one of claims 1-34, wherein the NSUN1/2-ACS inhibitor functions in a dose-dependent manner.

36. The method of any one of claims 1-34, comprising administering the NSUN1/2-ACS inhibitor at a concentration of 0.001 to 1,000 M/kg weight of the individual.

37. The method of any one of claims 1-36, comprising administering the NSUN1/2-ACS inhibitor at a concentration of 1 to 100 M/kg weight of the individual.

38. The method of any one of claims 1-37, comprising administering the NSUN1/2-ACS inhibitor at a concentration of 1 to 10 M/kg weight of the individual.

39. The method of any one of claims 1-38, wherein the NSUN1/2-ACS inhibitor comprises a compound of formula I 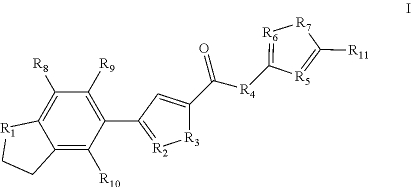
wherein:
R₁ is CH₂, NH, O, or S;
R₂ is CH or N;
R₃ is CH₂, NH, O, or S;
R₄ is NH or CH₂;
R₅ is N or CH;
R₆ is N or CH;
R₇ is CH₂, NH, O, or S;
R₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
R₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

40. The method of claim 39, wherein R₁ is O.

41. The method of claim 39, wherein R₄ is NH.

42. The method of claim 39, wherein R₁ is alkyl.

43. The method of claim 42, wherein R₁ is isopropyl.

44. The method of claim 39, wherein R₃ and R₇ are NH.

45. The method of claim 39, wherein R₂, R₅, and R₆ are N.

46. The method of claim 39, wherein the compound of formula I is further defined as

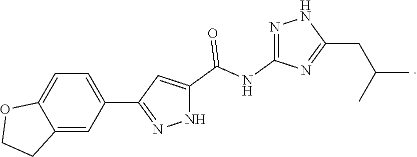 [SMILES: CC(C)Cc1nc(NC(=O)c2cc(-c3ccc4c(c3)CCO4)n[nH]2)n[nH]1]

47. The method of any one of claims 1-38, wherein the NSUN1/2-ACS inhibitor comprises a compound of formula IV: 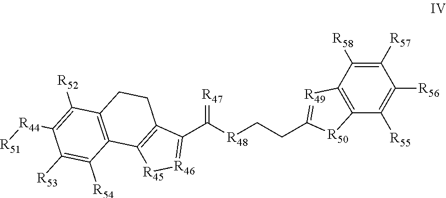
wherein:
R₄₄ is O, S, NH, or CH₂
R₄₅ is CH₂, NH, O, or S
R₄₆ is CH or N;
R₄₇ is NH, N-alkyl, O, or S;
R₄₈ is CH₂ or NH;
R₄₉ is CH or N;
R₅₀ is CH₂, NH, O, or S;
R₅₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
R₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

48. The method of claim 47, wherein R₄₅, R₄₈, and R₅₀ are NH.

49. The method of claim 47, wherein R₄₄ and R₄₇ are O.

50. The method of claim 47, wherein R₅₁ and R₅₇ are alkyl.

51. The method of claim 47, wherein R₅₁ is methyl.

52. The method of claim 47, wherein R₅₇ is methyl.

53. The method of claim 47, wherein the compound of formula IV is further defined as

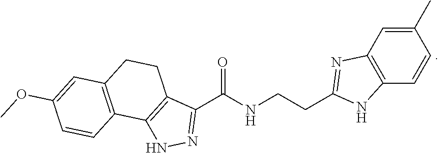

54. The method of any one of claims 1-38, wherein the NSUN1/2-ACS inhibitor comprises a compound of formula V: 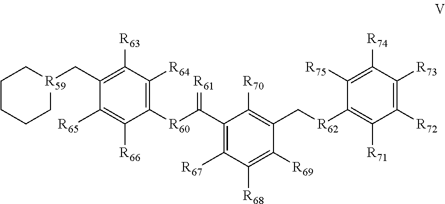
wherein:
R₅₉ is CH or N;
R₆₀ is CN₂, NH, O, or S;
R₆₁ is O or S;
R₆₂ is CH₂, NH, O, or S;
R₆₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

55. The method of claim 54, wherein R₅₉ is N.

56. The method of claim 54, wherein R₆₀ is NH.

57. The method of claim 54, wherein R₅₁ is a halogen.

58. The method of claim 57, wherein R₅₁ is —Br.

59. The method of claim 54, wherein R₆₂ is O.

60. The method of claim 54, wherein the compound of formula V is further defined as

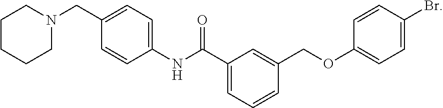 [SMILES: O=C(Nc1ccc(CN2CCCCC2)cc1)c1cccc(COc2ccc(Br)cc2)c1]

61. A method of overcoming and/or preventing cancer cell drug resistance to venetoclax-based and/or 5-azacytidine-based therapies comprising, contacting the cancer cell with one or more inhibitors of NSUN1 and/or NSUN2 interactions with Active Chromatin Site (ACS) associated proteins and/or complexes (NSUN1/2-ACS inhibitor).

62. The method of claim 61, wherein overcoming and/or preventing cancer cell drug resistance comprises administration of any one or more NSUN1/2-ACS inhibitors described in Tables 2-6.

63. A method of treating refractory cancer comprising, treatment with an NSUN1 and/or NSUN2 protein-protein interaction inhibitor with a NSUN1/2-ACS inhibitor.

64. The method of claim 63, wherein treating comprises administration of any one or more NSUN1/2-ACS inhibitors described in Tables 2-6 and/or a derivative thereof, and/or optionally wherein the method does not comprise administration of any one or more of NSUN1-38, NSUN1-33, NSUN1-36, NSUN1-28, NSUN1-4, NSUN1-32, NSUN1-43, NSUN1-44, NSUN1-46, NSUN1-41, NSUN2-43, NSUN2-51, NSUN2-41, NSUN2-29, NSUN2-23, NSUN2-55, NSUN2-22, NSUN2-26, NSUN2-2, NSUN2-52, NSUN2-49, NSUN2-56, NSUN2-66, NSUN2-40, and/or NSUN2-95.

65. A composition comprising NSUN2-11, NSUN2-37, NSUN1-9, NSUN1-10, NSUN1-11, NSUN1-17, NSUN1-17.13, NSUN1-17.16, NSUN1-24, NSUN1-25, NSUN1-27, NSUN1-30, NSUN1-31, NSUN1-54, NSUN1-64, NSUN1-67, NSUN1-73, NSUN1-74, NSUN2-3, NSUN2-10, NSUN2-17, NSUN2-18, NSUN2-19, NSUN2-20, NSUN2-21, NSUN2-35, NSUN2-38, NSUN2-39, NSUN2-64, NSUN2-98, and/or a derivative thereof, and a pharmaceutically acceptable excipient.

66. The composition of claim 65, wherein the composition comprises a derivative of compound NSUN1-9, NSUN1-10, NSUN1-11, NSUN1-17, NSUN1-17.13, NSUN1-17.16, NSUN1-24, NSUN1-25, NSUN1-27, NSUN1-30, NSUN1-31, NSUN1-54, NSUN1-64, NSUN1-67, NSUN1-73, NSUN1-74, NSUN2-3, NSUN2-10, NSUN2-11, NSUN2-17, NSUN2-18, NSUN2-19, NSUN2-20, NSUN2-21, NSUN2-35, NSUN2-37, NSUN2-38, NSUN2-39, NSUN2-64, and/or NSUN2-98.

67. The composition of claim 66, wherein the derivative is a water soluble derivative.

68. The composition of claim 65, comprising NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-11, NSUN2-37, NSUN2-35, and/or a derivative thereof.

69. The composition of claim 68, wherein the composition comprises a derivative of compound NSUN1-24, NSUN1-11, NSUN1-14, NSUN1-16, NSUN1-17, NSUN1-17.3, NSUN1-17.16, NSUN1-19, NSUN1-64, NSUN1-67, NSUN1-73, NSUN1-74, NSUN2-11, NSUN2-37, NSUN2-35, and/or a derivative thereof.

70. The composition of claim 69, wherein the derivative is a water soluble derivative.

71. The composition of claim 65, comprising NSUN1-17.16, NSUN1-11, NSUN1-67, NSUN2-11, NSUN2-37, NSUN2-35, and/or a derivative thereof.

72. The composition of claim 71, wherein the composition comprises a derivative of compound NSUN2-11, NSUN2-37, NSUN1-17.16, NSUN1-11, NSUN1-67, NSUN2-37, and/or NSUN2-35.

73. The composition of claim 72, wherein the derivative is a water soluble derivative.

74. The composition of any one of claims 65-73, comprising, consists essentially of, or consisting of NSUN1-11 and/or a derivative thereof.

75. The composition of any one of claims 65-73, comprising, consists essentially of, or consisting of NSUN1-67 and/or a derivative thereof.

76. The composition of any one of claims 65-73, comprising, consists essentially of, or consisting of NSUN1-17.16 and/or a derivative thereof.

77. The composition of any one of claims 65-73, comprising, consists essentially of, or consisting of NSUN2-11 and/or a derivative thereof.

78. The composition of any one of claims 65-73, comprising, consists essentially of, or consisting of NSUN2-37 and/or a derivative thereof.

79. The composition of any one of claims 65-73, comprising, consists essentially of, or consisting of NSUN2-35 and/or a derivative thereof.

80. The composition of any one of claims 65-79, further comprising venetoclax and/or 5-azacytidine.

81. A composition comprising a compound of formula I 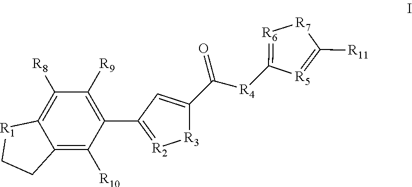
wherein:
R₁ is CH₂, NH, O, or S;
R₂ is CH or N;
R₃ is CH₂, NH, O, or S;
R₄ is NH or CH₂;
R₅ is N or CH;
R₆ is N or CH;
R₇ is CH₂, NH, O, or S;
R₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₁₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
R₁₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

82. The composition of claim 81, wherein R₁ is O.

83. The composition of claim 81, wherein R₄ is NH.

84. The composition of claim 81, wherein R₁₁ is alkyl.

85. The composition of claim 81, wherein R₁₁ is isopropyl.

86. The composition of claim 81, wherein R₃ and R₇ are NH.

87. The composition of claim 81, wherein R₂, R₅, and R₆ are N.

88. The composition of claim 81, wherein the compound of formula I is further defined as

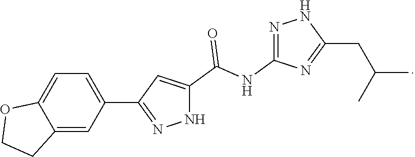 [SMILES: CC(C)Cc1nc(NC(=O)c2cc(-c3ccc4c(c3)CCO4)n[nH]2)n[nH]1]

89. A composition comprising a compound of formula IV: 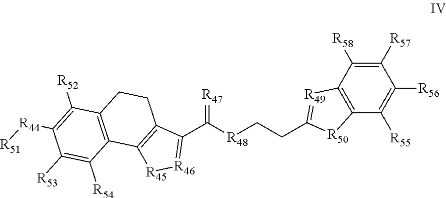
wherein:
R₄₄ is O, S, NH, or CH₂
R₄₅ is CH₂, NH, O, or S
R₄₆ is CH or N;
R₄₇ is NH, N-alkyl, O, or S;
R₄₈ is CH₂ or NH;
R₄₉ is CH or N;
R₅₀ is CH₂, NH, O, or S;
R₅₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₅₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide; and
R₅₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

90. The composition of claim 89, wherein R₄₅, R₄₈, and R₅₀ are NH.

91. The composition of claim 89, wherein R₄₄ and R₄₇ are O.

92. The composition of claim 89, wherein R₅₁ and R₅₇ are alkyl.

93. The composition of claim 89, wherein R₅₁ is methyl.

94. The composition of claim 89, wherein R₅₇ is methyl.

95. The composition of claim 89, wherein the compound of formula IV is further defined as

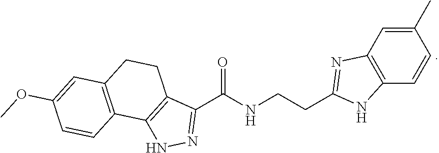

96. A composition comprising a compound of formula V: 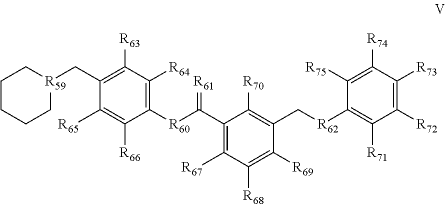
wherein:
R₅₉ is CH or N;
R₆₀ is CN₂, NH, O, or S;
R₆₁ is O or S;
R₆₂ is CH₂, NH, O, or S;
R₆₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₆ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₇ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₈ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₆₉ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₀ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₁ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₂ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₃ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₄ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
R₇₅ is H, halogen, hydroxyl, alkoxy, thiol, thioalkyl, ester, ether, epoxide, amine, amide, alkyl, heteroalkyl, cycloalkyl, heterocyclyl, aryl, heteroaryl, amine, alkylamine, sulfoxide, sulfone, sulfinic acid, sulfonic acid, sulfonate ester, phosphine, phosphate, phosphate ester, aldehyde, carboxylic acid, nitrate, nitrite, nitrile, isocyanate, or azide;
or an enantiomer, diastereomer, racemic mixture, or salt thereof.

97. The composition of claim 96, wherein R₅₉ is N.

98. The composition of claim 96, wherein R₆₀ is NH.

99. The composition of claim 96, wherein R₅₁ is a halogen.

100. The composition of claim 96, wherein R₅₁ is —Br.

101. The composition of claim 96, wherein R₆₂ is O.

102. The composition of claim 96, wherein the compound of formula V is further defined as 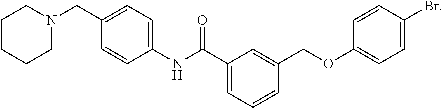 [SMILES: O=C(Nc1ccc(CN2CCCCC2)cc1)c1cccc(COc2ccc(Br)cc2)c1]
